SEQUENCE LISTING (72 sequences)

SEQ ID NO: 1 - DNA, 20 bp, Artificial Sequence
  Rspo2 forward primer
agaggccgttgctttgatga

SEQ ID NO: 2 - DNA, 20 bp, Artificial Sequence
  Rspo2 reverse primer
tccccattcgctccaatgac

SEQ ID NO: 3 - DNA, 18 bp, Artificial Sequence
  GAPDH forward primer
gaaggtgaaggtcggagt

SEQ ID NO: 4 - DNA, 20 bp, Artificial Sequence
  GAPDH reverse primer
gaagatggtgatgggatttc

SEQ ID NO: 5 - DNA, 43 bp, Artificial Sequence
  Rspo2 exon 2 gene specific primer
gattacgccaagcttcgtctccatcggttgccttggcagtggc

SEQ ID NO: 6 - DNA, 43 bp, Artificial Sequence
  Rspo2 exon3 gene specific primer
gattacgccaagcttgcaggcactctccatactggcgcatccc

SEQ ID NO: 7 - DNA, 44 bp, Artificial Sequence
  Rspo2 exon 3 nested primer
gattacgccaagcttgggctcggtgtccatagtacccggatggg

SEQ ID NO: 8 - DNA, 42 bp, Artificial Sequence
  Rspo3 exon 3 gene specific primer
gattacgccaagcttggttgttggcttccaacccttctgggc

SEQ ID NO: 9 - DNA, 46 bp, Artificial Sequence
  Rspo3 exon3 nested gene specific primer
gattacgccaagcttggacccgtgtttcagtccctcttttgaagcc

SEQ ID NO: 10 - DNA, 85 bp, Artificial Sequence
  NM_001101.21010_T001 capture probe
gatcttgatcttcattgtgctgggtgccagggcagtgatctccttctgcacctcaagacctaagcgacagcgtgaccttgtttca

SEQ ID NO: 11 - DNA, 74 bp, Artificial Sequence
  NM_002046.3972_T002 capture probe
aagtggtcgttgagggcaatgccagccccagcgtcaaagcatcctcttcttttcttggtgttgagaagatgctc

SEQ ID NO: 12 - DNA, 85 bp, Artificial Sequence
  NM_004655.3527_T003 capture probe
tcggaacaggtaagcaccgtcttgatcgcccaataaggagtgtaaggactcacaattctgcgggttagcaggaaggttagggaac

SEQ ID NO: 13 - DNA, 85 bp, Artificial Sequence
  NM_003667.33414_T004 capture probe
gaaaacccttattttcctaattcccccaacaacctcttgagtttgtgggtctgttgagattattgagcttcatcatgaccagaag

SEQ ID NO: 14 - DNA, 79 bp, Artificial Sequence
  NM_178565.4150_T005 capture probe
ttcctgcgctttctccacggtcacttcacagctaagatttctttcaaagacgcctatcttccagtttgatcgggaaact

SEQ ID NO: 15 - DNA, 85 bp, Artificial Sequence
  NM_178565.4641_T006 capture probe
gatgagggcaaaggagaaaaggcgaaactgcatctgggcggtcgggcgggcgaacctaactcctcgctacattcctattgttttc

SEQ ID NO: 16 - DNA, 85 bp, Artificial Sequence
  NM_178565.41060_T007 capture probe
ctccaatgaccaacttcacatccttccacacattccatggtttcttctaaccaatttggttttactcccctcgattatgcggagt

SEQ ID NO: 17 - DNA, 85 bp, Artificial Sequence
  NM_178565.42289_T008 capture probe
gttgtgtttcatcaaagtcatcacatcttagactcaaaggcagtttgggcctttcgggttatatctatcatttacttgacaccct

SEQ ID NO: 18 - DNA, 85 bp, Artificial Sequence
  Fusion_0622.169_T009 capture probe
cctcctttacagagagaaattcaagaagcggaaagactagatgccgatcccaacagccactttttttccaaattttgcaagagcc

SEQ ID NO: 19 - DNA, 85 bp, Artificial Sequence
  Fusion_0623.133_T010 capture probe
ccacgaaccttcccaagtgacatcgtaaagctctgagaccttcgccatctcaccgtgtggacggcaactcagagataacgcatat

SEQ ID NO: 20 - DNA, 85 bp, Artificial Sequence
  Fusion_0625.177_T011 capture probe
aaggctgtggtagggaacggagtctatatttgtagagaagagtagaatcacctggagtttatgtattgccaacgagtttgtcttt

SEQ ID NO: 21 - DNA, 70 bp, Artificial Sequence
  Fusion_0624.1155_T012 capture probe
aactggagggcagatctggccgtgtctccacaggtcagataaggttgttattgtggaggatgttactaca

SEQ ID NO: 22 - DNA, 85 bp, Artificial Sequence
  NM_032784.4173_T013 capture probe
taaaatatatgtaggtgttaggcgtatatagacagtgcccgagcagcgggcttccttcctgtgttccagctacaaacttagaaac

SEQ ID NO: 23 - DNA, 85 bp, Artificial Sequence
  NM_032784.4634_T014 capture probe
cttctgggcaattgtcaaggcactttccaaggtgtaagtaaaatccacttcataaaattggttttgcctttcagcaattcaactt

SEQ ID NO: 24 - DNA, 85 bp, Artificial Sequence
  NM_032784.41520_T015 capture probe
ctcccggtcaaaacaacactgtcctttgaaggatgtttctcttccctgaactggtcaagacttgcatgaggacccgcaaattcct

SEQ ID NO: 25 - DNA, 84 bp, Artificial Sequence
  Fusion_0169.11_T016 capture probe
cttggggcatctcgggtggtagataatgaggctcttgagcactaacatgctttcgttgggacgcttgaagcgcaagtagaaaac

SEQ ID NO: 26 - DNA, 85 bp, Artificial Sequence
  Fusion_0736.10_T017 capture probe
cacaaggataactcagtacttggatgtcatcaatggcaatataaccacttccagcagacctgcaatatcaaagttataagcgcgt

SEQ ID NO: 27 - DNA, 84 bp, Artificial Sequence
  Fusion_0170.11_T018 capture probe
ctctcacaataagttgagcaaaattggacacaccggaacctcgttctgacctgccaatgcactcgatcttgtcatttttttgcg

SEQ ID NO: 28 - DNA, 84 bp, Artificial Sequence
  Fusion_0737.10_T019 capture probe
catcttcatcagtttgaataattgtctcttcactctccttccttccctccaaactggagagagaagtgaagacgatttaaccca

SEQ ID NO: 29 - DNA, 70 bp, Artificial Sequence
  Fusion_0524.10_T020 capture probe
ctgcggagaactggccttgggccgatcccaggagacgattgctgcattccgctcaacgcttgaggaagta

SEQ ID NO: 30 - DNA, 75 bp, Artificial Sequence
  Fusion_0525.10_T021 capture probe
ctgcacattttgttctggtgattagtggaggtcctgggagctgaggctgttaaagctgtagcaactcttccacga

SEQ ID NO: 31 - DNA, 85 bp, Artificial Sequence
  Fusion_0526.10_T022 capture probe
cttttgtagcaatgcgtacgcactgggttttagtttccttctccacactgctaggacgcaaatcacttgaagaagtgaaagcgag

SEQ ID NO: 32 - DNA, 85 bp, Artificial Sequence
  Fusion_0684.10_T023 capture probe
ctccttttcacttgtcatttcagtaactgcaggccttttctgcggagaacccacgcgatgacgttcgtcaagagtcgcataatct

SEQ ID NO: 33 - DNA, 85 bp, Artificial Sequence
  Fusion_0738.10_T024 capture probe
ctgagatattggtggtgacattgatggctgtggctggaccaaagcctttgcatttggaatgatgtgtactgggaataagacgacg

SEQ ID NO: 34 - DNA, 75 bp, Artificial Sequence
  NM_001101.21010_ProbeB reporter probe
cgaaagccatgacctccgatcactcaggatggagccgccgatccacacggagtacttgcgctcaggaggagcaat

SEQ ID NO: 35 - DNA, 75 bp, Artificial Sequence
  NM_002046.3972_ProbeB reporter probe
cgaaagccatgacctccgatcactcccctgttgctgtagccaaattcgttgtcataccaggaaatgagcttgaca

SEQ ID NO: 36 - DNA, 75 bp, Artificial Sequence
  NM_004655.3527_ProbeB reporter probe
cgaaagccatgacctccgatcactcgcaaaccagaagtctaaggtatccacgcatttctccctctccaggaaagt

SEQ ID NO: 37 - DNA, 75 bp, Artificial Sequence
  NM_003667.33414_ProbeB reporter probe
cgaaagccatgacctccgatcactctagaatgaaatcccatggatcacagcctctacctagcaatgtaggtcatt

SEQ ID NO: 38 - DNA, 62 bp, Artificial Sequence
  NM_178565.4150_ProbeB reporter probe
cgaaagccatgacctccgatcactccgctcacactctctgcctccctaaccaattgtgtcgc

SEQ ID NO: 39 - DNA, 68 bp, Artificial Sequence
  NM_178565.4641_ProbeB reporter probe
cgaaagccatgacctccgatcactcatcggttgccttggcagtggctgtaatccatgcagttcagaat

SEQ ID NO: 40 - DNA, 75 bp, Artificial Sequence
  NM_178565.41060_ProbeB reporter probe
cgaaagccatgacctccgatcactccccatttaaatccacatgtgcgattatttctgctacaagttccccattcg

SEQ ID NO: 41 - DNA, 75 bp, Artificial Sequence
  NM_178565.42289_ProbeB reporter probe
cgaaagccatgacctccgatcactcggcctgtgaaactggctacaagtgactggatatagtccctcatgttttca

SEQ ID NO: 42 - DNA, 63 bp, Artificial Sequence
  Fusion_0622.169_ProbeB reporter probe
cgaaagccatgacctccgatcactccggtcagttcagcgcgatcagcatctctccgccacgaa

SEQ ID NO: 43 - DNA, 61 bp, Artificial Sequence
  Fusion_0623.133_ProbeB reporter probe
cgaaagccatgacctccgatcactccgcaccggtcagttcagcgcgatcagcatctctccg

SEQ ID NO: 44 - DNA, 64 bp, Artificial Sequence
  Fusion_0625.177_ProbeB reporter probe
cgaaagccatgacctccgatcactccggtcagttcagcgcgatcagcatctctccgccacgaac

SEQ ID NO: 45 - DNA, 61 bp, Artificial Sequence
  Fusion_0624.1155_ProbeB reporter probe
cgaaagccatgacctccgatcactccggtcagttcagcgcgatcagcatctctccgccacg

SEQ ID NO: 46 - DNA, 75 bp, Artificial Sequence
  NM_032784.4173_ProbeB reporter probe
cgaaagccatgacctccgatcactctcccttcctttctcctctttcttttgattgttaattatatttaatgtttt

SEQ ID NO: 47 - DNA, 75 bp, Artificial Sequence
  NM_032784.4634_ProbeB reporter probe
cgaaagccatgacctccgatcactcacagtgcacaatactgacacactccatagtatggttgttggcttccaacc

SEQ ID NO: 48 - DNA, 75 bp, Artificial Sequence
  NM_032784.41520_ProbeB reporter probe
cgaaagccatgacctccgatcactcaaatcctgtgattccaaatgccaggccctaattctgagcactctctagat

SEQ ID NO: 49 - DNA, 64 bp, Artificial Sequence
  Fusion_0169.11_ProbeB reporter probe
cgaaagccatgacctccgatcactccacagcctccttggcagccttgactaacgttaggatgca

SEQ ID NO: 50 - DNA, 64 bp, Artificial Sequence; identical to SEQ ID NO: 49
  Fusion_0736.10_ProbeB reporter probe

SEQ ID NO: 51 - DNA, 64 bp, Artificial Sequence; identical to SEQ ID NO: 49
  Fusion_0170.11_ProbeB reporter probe

SEQ ID NO: 52 - DNA, 64 bp, Artificial Sequence; identical to SEQ ID NO: 49
  Fusion_0737.10_ProbeB reporter probe

SEQ ID NO: 53 - DNA, 64 bp, Artificial Sequence; identical to SEQ ID NO: 49
  Fusion_0524.10_ProbeB reporter probe

SEQ ID NO: 54 - DNA, 64 bp, Artificial Sequence; identical to SEQ ID NO: 49
  Fusion_0525.10_ProbeB

SEQ ID NO: 55 - DNA, 64 bp, Artificial Sequence; identical to SEQ ID NO: 49
  Fusion_0526.10_ProbeB reporter probe

SEQ ID NO: 56 - DNA, 64 bp, Artificial Sequence; identical to SEQ ID NO: 49
  Fusion_0684.10_ProbeB reporter probe

SEQ ID NO: 57 - DNA, 64 bp, Artificial Sequence; identical to SEQ ID NO: 49
  Fusion_0738.10_ProbeB reporter probe

SEQ ID NO: 58 - DNA, 100 bp, Artificial Sequence
  PTPRKe1-Rspo3e2 gene fusion
cctctctccttggcctctcctgggatcggcccaaggccagttctccgcagtgcatcctaacgttagtcaaggctgccaaggaggctgtgcaacatgctca

SEQ ID NO: 59 - DNA, 100 bp, Artificial Sequence
  PTPRKe7-Rspo3e2 gene fusion
tggaacggggctcccaggacctccactaatcaccagaacaaaatgtgcagtgcatcctaacgttagtcaaggctgccaaggaggctgtgcaacatgctca

SEQ ID NO: 60 - DNA, 100 bp, Artificial Sequence
  PTPRKe6X-Rspo3e2 gene fusion
gttctccgcagaaaaggcctgcagttactgaaatgacaagtgaaaaggagtgcatcctaacgttagtcaaggctgccaaggaggctgtgcaacatgctca

SEQ ID NO: 61 - DNA, 100 bp, Artificial Sequence
  PTPRKe13-Rspo3e2 gene fusion
cagtgtggagaaggaaactaaaacccagtgcgtacgcattgctacaaaagtgcatcctaacgttagtcaaggctgccaaggaggctgtgcaacatgctca

SEQ ID NO: 62 - DNA, 100 bp, Artificial Sequence
  EIF3Ee1-Rspo2e2 gene fusion
ggatcggcatctagtctttccgcttcttgaatttctctctgtaaaggaggttcgtggcggagagatgctgatcgcgctgaactgaccggtgcggcccggg

SEQ ID NO: 63 - DNA, 100 bp, Artificial Sequence
  EIF3Ee1-Rspo2e3 gene fusion
tggatcggcatctagtctttccgcttcttgaatttctctctgtaaaggagctagttatgtatcaaatcccatttgcaagggttgtttgtcttgttcaaag

SEQ ID NO: 64 - DNA, 100 bp, Artificial Sequence
  EMC2e1-Rspo2e2 gene fusion
agatggcgaaggtctcagagctttacgatgtcacttgggaaggttcgtggcggagagatgctgatcgcgctgaactgaccggtgcggcccgggggtgagt

SEQ ID NO: 65 - DNA, 100 bp, Artificial Sequence
  PVT1-Rspo2e2 gene fusion
ttggcggtccctgtgacctgtggagacacggccagatctgccctccagttcgtggcggagagatgctgatcgcgctgaactgaccggtgcggcccggggg

SEQ ID NO: 66 - DNA, 97 bp, Artificial Sequence
  PVT1-Rspo2e3 gene fusion
ttggcggtccctgtgacctgtggagacacggccagatctgccctccactagttatgtatcaaatcccatttgcaagggttgtttgtcttgttcaaag

SEQ ID NO: 67 - DNA, 100 bp, Artificial Sequence
  HNF4G-Rspo2e2 gene fusion
tgattctactcttctctacaaatatagactccgttccctaccacagccttgttcgtggcggagagatgctgatcgcgctgaactgaccggtgcggcccgg

SEQ ID NO: 68 - DNA, 132 bp, Artificial Sequence
  PTPRKex2-RSPO3ex2 gene fusion
atgatgactttgaatgggtgcatgttagtgctcaagagcctcattatctaccacccgagatgccccaagtgcatcctaacgttagtcaaggctgccaaggaggctgtgcaacatgctcagattacaatggat

SEQ ID NO: 69 - DNA, 126 bp, Artificial Sequence
  PTPRKex4-RSPO3ex2 gene fusion
tctcaggagggagaagtggttatattgccattgatgacatccaagtactgagttatccttgtgtgcatcctaacgttagtcaaggctgccaaggaggctgtgcaacatgctcagattacaatggat

SEQ ID NO: 70 - DNA, 127 bp, Artificial Sequence
  PTPRKex6-RSPO3ex2 gene fusion
cgctgtgtaactcagtcagaacgaggttccggtgtgtccaattttgctcaacttattgtgagagtgcatcctaacgttagtcaaggctgccaaggaggctgtgcaacatgctcagattacaatggat

SEQ ID NO: 71 - DNA, 124 bp, Artificial Sequence
  PTPRKex8-RSPO3ex2 gene fusion
ctaaccaatccagagggaaggaaggagagtgaagagacaattattcaaactgatgaagatgtgcatcctaacgttagtcaaggctgccaaggaggctgtgcaacatgctcagattacaatggat

SEQ ID NO: 72 - DNA, 126 bp, Artificial Sequence
  PTPRKex10-RSPO3ex2 gene fusion
gagccagcacggtcaaaggctttggtccagccacagccatcaatgtcaccaccaatatctcagtgcatcctaacgttagtcaaggctgccaaggaggctgtgcaacatgctcagattacaatggat

(19) United States
(12) Patent Application Publication
(10) Pub. No.: US 2021/0054466 A1
(43) Pub. Date: Feb. 25, 2021

(54) TUMOR BIOMARKERS AND USE THEREOF

(71) Applicant: Curegenix Corporation, Burlingarne, CA (US)

(72) Inventors: Xiaoli Qin, Burlingame, CA (US); Songzhu An, Burlingame, CA (US); Tao Huang, Burlingame, CA (US)

(21) Appl. No.: 17/092,653

(22) Filed: Nov. 9, 2020

Related U.S. Application Data
(62) Division of application No. 15/575,780, filed on Nov. 20, 2017.
(60) Provisional application No. 62/166,305, filed on May 26, 2015.

(51) Int. Cl. C12Q 1/6886; A61K 31/444; A61K 31/4985; A61K 31/4725; G01N 33/574; A61P 35/00

(52) U.S. Cl. CPC C12Q 1/6886; A61K 31/444; A61K 31/4985; C12Q 2600/158; G01N 33/57484; A61P 35/00; G01N 2800/52; A61K 31/4725

(57) ABSTRACT

Disclosed herein are biomarkers related to WNT signal transduction pathway, as well as methods and kits comprising the same. Further, the present disclosure relates to the use of the biomarkers in patient selection, companion diagnostics, and treatment of cancer.

### CROSS REFERENCE TO RELATED APPLICATIONS

This application is a divisional of U.S. patent application Ser. No. 15/575,780, filed Nov. 20, 2017, which is a 35 U.S.C. 371 national phase entry of PCT/US2016/034245, filed May 26, 2016, which claims the benefit of, and priority to, U.S. Provisional Patent Application Ser. No. 62/166,305, filed on May 26, 2015, the entire disclosures of which are hereby incorporated by reference in their entirety.

### FIELD OF THE INVENTION

The present invention relates to biomarkers related to WNT signal transduction pathway, as well as methods and kits comprising the same. Further, the present invention relates to the use of the biomarkers in patient selection, companion diagnostics, and treatment of cancer.

### BACKGROUND OF THE INVENTION

Cancer is a class of diseases that affects people world-wide. Generally, cells in a benign tumor retain their differentiated features and do not divide in a completely uncontrolled manner. A benign tumor is usually localized and non-metastatic.

In a malignant tumor, cells become undifferentiated, do not respond to the body's growth control signals, and multiply in an uncontrolled manner. Malignant tumors are generally divided into two categories: primary and secondary. Primary tumors arise directly from the tissue in which they are found. Secondary tumors may be originated from the primary tumors or may be originated elsewhere in the body, and are capable of spreading to distant sites (metastasizing) or metastasis. The common routes for metastasis are direct growth into adjacent structures, spread through the vascular or lymphatic systems or blood streams.

WNT signaling is important to both embryogenesis and homeostasis in adult animals. The WNT pathway is comprised in general of a network of proteins that regulate the following processes: (1) the production and secretion of WNT proteins; (2) the binding of WNT with cellular receptors; and (3) the intracellular transduction of the biochemical responses triggered by the interaction (Mikels and Nusse, 2006; MacDonald, 2009; Moon, 2005).

The so-called canonical WNT pathway triggered by binding of WNT proteins to cell surface co-receptors Frizzled LRP5/6 results in a change in the amount of β-catenin that reaches the nucleus where it interacts with TCF/LEF family transcription factors to promote transcription of specific genes.

The non-canonical WNT pathway transduced by a different set of intracellular proteins controls planar cell polarity in insects and several processes such as gastrulation in vertebrates.

WNT signaling is also known for its roles in controlling pluripotency and differentiation of embryonic and adult stem cells (Nusse, 2008). For example, formation of the primitive streak during gastrulation was associated with localized WNT activation in the embryoid bodies (Ten Berge, 2008). The derivation of a number of cell types, such as heart cells, pancreatic beta cells, dopminergic neurons and liver hepatocytes from embryonic stem cells or PS cells is influenced by WNT modulation (Yang, 2008; D'Amour, 2006; Inestrosa and Arenas, 2010; Sullivan, 2010). The WNT pathway plays a particularly important role in skeletal tissue development such as osteogenesis and chondrogenesis (Hoeppner, 2009; Chun, 2008). WNT signaling is also associated with neuro-regeneration of the adult central nervous system (Lie, 2005).

Diseases may arise from altered WNT pathway activity. For example, hyperactivation of the canonical WNT pathway can lead to aberrant cell growth (Reya and Clevers, 2005). Notably, 90% of colorectal cancers are initiated by the loss of the adenomatosis polyposis coli (APC) gene, a suppressor of the WNT/β-catenin pathway (Kinzler and Vogelstein, 1996). Increased expression of WNT proteins and loss of extracellular inhibitors that normally suppress WNT protein function may give rise to WNT-dependent tumors (Polakis, 2007). On the other hand, the non-canonical WNT pathway has also been shown to play a role in the progression of certain cancers (Camilli and Weeraratna, 2010). More recently, WNT signaling is also implicated in cancer stem cells (Takahashi-Yanaga and Kahn, 2010).

Evidence suggests that targeting the Wnt-mediated signal transduction pathway would be therapeutically useful in a broad range of diseases (Barker and Clevers, 2006). Mutations of APC, beta-catenin or axin-1 leading to constitutive activation of the canonical Wnt pathway are critical events in a variety of human cancers including colorectal cancer, melanoma, hepatocellular carcinoma, gastric cancer, ovarian cancer and others (Polakis, 2007). Blockade of the Wnt pathway in a variety of cancers using either genetic or chemical approaches has been shown to abrogate aberrant cell growth (Herbst and Kolligs, 2007). Furthermore, inhibition of this pathway may directly influence the cells that sustain cancer cell growth and enable metastasis, and that are thought to be resistant to traditional chemotherapeutic agents.

In addition to activation caused by mutations of gene products downstream of the receptors, aberrant Wnt pathway activity caused by other mechanisms have been associated with a broad range of cancers. These cancers include but not limited to: lung (small cell and non-small cell), breast, prostate, carcinoid, bladder, scarcinoma, esophageal, ovarian, cervical, endometrial, mesothelioma, melanoma, sarcoma, osteosarcoma, liposarcoma, thyroid, desmoids, chronic myelocytic leukemia (AML), and chronic myelocytic leukemia (CML). There are now multiple examples of cancer cells dependent upon upregulated autocrine or paracrine Wnt signaling, and cell lines from osteosarcoma, breast, head and neck and ovarian cancers have been shown to derive protection from apoptosis by autocrine or paracrine Wnt signaling (Kansara, 2009; Bafico, 2004; Aki, 2009; DeAlmeida, 2007; Chan, 2007; Chen, 2009; Rhee, 2002).

Furthermore, aberrant Wnt pathway has been implicated in the development of fibrosis, include but are not limited to: lung fibrosis, such as idiopathic pulmonary fibrosis and radiation-induced fibrosis, renal fibrosis and liver fibrosis (Morrisey, 2003; Hwang, 2009; Cheng, 2008).

Other disorders associated with aberrant WNT signaling, include but are not limited to bone and cartilage disorders, such as osteoporosis and osteoarthritis, obesity associated type II diabetes, and neurodegenerative diseases such as Alzheimer's disease (Hoeppner, 2009; Ouchi, 2010; Blom, 2010; Boonen, 2009). WNT signaling also contributes to the self-renewal and maintenance of HSC's, and dysfunctional WNT signaling is responsible for various disorders resulting from HSC's, such as leukemias and various other blood related cancers (Reya, 2005).

### SUMMARY OF THE INVENTION

The present invention generally provides biomarkers related WNT pathway, and the use of such biomarker in patient selection for treatment of diseases, such as cancer.

In one aspect, the present invention provides a method for treating cancer characterized by expression of an R-spondin fusion in a subject that has been diagnosed with cancer and is in need of such treatment, comprising: administering to a subject diagnosed with cancer a pharmaceutical composition comprising a therapeutically effective amount of an antagonist of Porcupine, wherein said subject has been determined to have an R-spondin fusion.

In some embodiments, the R-spondin fusion comprising: (1) a PTPRKe1-Rspo3e2 fusion; (2) a PTPRKe7-Rspo3e2 fusion; (3) an EIF3Ee1-Rspo2e2 fusion; or (4) an EIF3Ee1-Rspo2e3 fusion.

In some embodiments, the R-spondin fusion comprising: (1) an EMC2e1-Rspo2e2 fusion (2) a PVT1-Rspo2e2 fusion; (3) a PVT1-Rspo2e3 fusion; (4) an HNF4G-Rspo2e2 fusion; (5) a PTPRKe13-Rspo3e2 fusion; or (6) a PTPRKe6X-Rspo3e2 fusion.

In some embodiments, the subject is determined to have R-spondin mRNA expression level that is higher than the R-spondin mRNA expression level in a control subject that has been determined that does not have a R-spondin fusion.

In some embodiments, the Rspondin fusion comprises a junction sequence of any one of SEQ ID NO.:58, SEQ ID.:59, SEQ ID NO.:62, or SEQ ID NO.:63.

In some embodiments, the EMC2e1-Rspo2e2 fusion comprises a junction sequence of SEQ ID NO.:64.

In some embodiments, the PVT1-Rspo2e2 fusion comprises a junction sequence of SEQ ID NO.:65.

In some embodiments, the PVT1-Rspo2e3 fusion comprises a junction sequence of SEQ ID NO.:66.

In some embodiments, the HNF4G-Rspo2e2 fusion comprises a junction sequence of SEQ ID NO.:67.

In some embodiments, the PTPRKe13-Rspo3e2 fusion comprises a junction sequence of SEQ ID NO.:61.

In some embodiments, the PTPRKe6X-Rspo3e2 fusion comprises a junction sequence of SEQ ID NO.:60.

In some embodiments, the Rspondin is Rspo2 or Rsp3, and the fusion gene is overexpressed in comparison to the Rspondin that is not fused to another gene.

In some embodiments, the Porcupine antagonist comprises a compound of Formula (I):

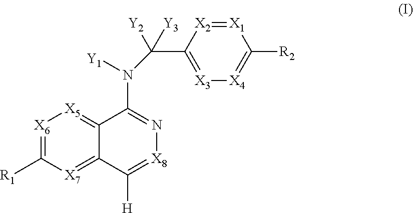

or a physiologically acceptable salt thereof, wherein
X₁, X₂, X₃, X₄, X₅, X₆, X₇, X₈ are independently CR₄ or N;
Y₁ is hydrogen or CR₄; Y₂, Y₃ are independent hydrogen, halo or CR₃;
R₁ is morpholinyl, piperazinyl, quinolinyl,

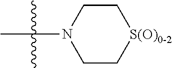

aryl, C₁₋₆ heterocycle, 5 or 6 membered heteroaryl containing 1-2 heteroatoms selected from N, O and S;
R₂ is hydrogen, halo, morpholinyl, piperazinyl, quinolinyl,

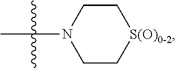

aryl, C₁₋₆ heterocycle, 5 or 6 membered heteroaryl containing 1-2 heteroatoms selected from N, O and S;
R₃ is hydrogen, halo, cyano, C₁₋₆ alkyl, C₁₋₆ alkoxy optionally substituted with halo, amino, hydroxyl, alkoxy or cyano;
R₄ is hydrogen, halo, C₁₋₆ alkoxy, —S(O)₂R₅, —C(O)OR₅, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano.

In some embodiments, the 5 or 6 membered heteroaryl is selected from:

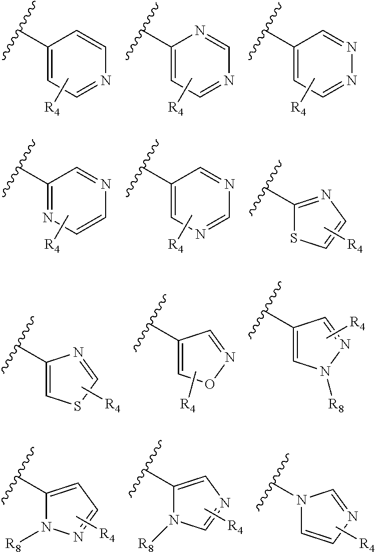

wherein,
R₄ is hydrogen, halo, C₁₋₆ alkoxy, —S(O)₂R₄, —C(O)OR₄, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano; and
R₈ is hydrogen or C₁₋₆ alkyl.

In some embodiments, R₁ and R₂ is independently substituted with 1 or 2 R₄ groups.

In some embodiments, the compound is selected from: 6-(2-methylpyridin-4-yl)-N-(4-(2-methylpyridin-4-yl)benzyl)-2,7-naphthyridin-1-amine;
N-(3-methyl-4-(2-methylpyridin-4-yl)benzyl)-6-(2-methylpyridin-4-yl)-2,7-naphthyridin-1-amine;
6-(3-fluorophenyl)-N-((2′-methyl-2,4′-bipyridin-5-yl)methyl)isoquinolin-1-amine;
2-(2-methylpyridin-4-yl)-N-(4-(2-methylpyridin-4-yl)benzyl)-1,6-naphthyridin-5-amine;
N-(4-(2-methylpyridin-4-yl)benzyl)-2-phenylpyrido[4,3-b]pyrazin-5-amine;
N-(4-(2-methylpyridin-4-yl)benzyl)-6-(pyridin-4-yl)-2,7-naphthyridin-1-amine;
N-(4-(2-methylpyridin-4-yl)benzyl)-6-phenyl-2,7-naphthyridin-1-amine;
6-(3-chlorophenyl)-N-(4-(2-methylpyridin-4-yl)benzyl)-2,7-naphthyridin-1-amine;
6-(3-fluorophenyl)-N-(4-(2-methylpyridin-4-yl)benzyl)-2,7-naphthyridin-1-amine;
6-(3-fluorophenyl)-N-((2′-methyl-2,4′-bipyridin-5-yl)methyl)-2,7-naphthyridin-1-amine;
6-(3-fluorophenyl)-N-(4-(2-(trifluoromethyl)pyridin-4-yl)benzyl)-2,7-naphthyridin-1-amine;
N-((2′,3-dimethyl-2,4′-bipyridin-5-yl)methyl)-6-(3-fluorophenyl)-2,7-naphthyridin-1-amine;
N-(4-(2-methylpyridin-4-yl)benzyl)-6-(pyrimidin-5-yl)-2,7-naphthyridin-1-amine;
6-(5-methylpyridin-3-yl)-N-(4-(2-methylpyridin-4-yl)benzyl)-2,7-naphthyridin-1-amine;
6-(6-methylpyridin-3-yl)-N-(4-(2-methylpyridin-4-yl)benzyl)-2,7-naphthyridin-1-amine;
3-(8-(4-(2-methylpyridin-4-yl)benzylamino)-2,7-naphthyridin-3-yl)benzonitrile;
4-(8-(4-(2-methylpyridin-4-yl)benzylamino)-2,7-naphthyridin-3-yl)benzonitrile;
6-(4-fluorophenyl)-N-(4-(2-methylpyridin-4-yl)benzyl)-2,7-naphthyridin-1-amine;
N-(4-(2-methylpyridin-4-yl)benzyl)-8-m-tolyl-2,7-naphthyridin-1-amine;
N-(4-(2-methylpyridin-4-yl)benzyl)-2,7-naphthyridin-1-amine;
N-(4-(2-methylpyridin-4-yl)benzyl)-6-(pyridin-2-yl)-2,7-naphthyridin-1-amine;
6-(2-fluoropyridin-4-yl)-N-(4-(2-methylpyridin-4-yl)benzyl)-2,7-naphthyridin-1-amine;
6-(2-fluorophenyl)-N-(4-(2-methylpyridin-4-yl)benzyl)-2,7-naphthyridin-1-amine;
N-(4-(2-methylpyridin-4-yl)benzyl)-6-(pyridin-3-yl)-2,7-naphthyridin-1-amine;
N-(biphenyl-4-ylmethyl)-6-(2-methylpyridin-4-yl)-2,7-naphthyridin-1-amine;
6-(2-methylpyridin-4-yl)-N-((5-phenylpyridin-2-yl)methy-2,7-naphthyridin-1-amine;
6-(3-fluorophenyl)-N-((2′-(trifluoromethyl)-2,4′-bipyridin-5-yl)methyl)-2,7-naphthyridin-1-amine;
N-(3-fluoro-4-(2-fluoropyridin-4-yl)benzyl)-6-(2-methylpyridin-4-yl)-2,7-naphthyridin-1-amine;
6-(2-methylpyridin-4-yl)-N-((2′-(trifluoromethyl)-2,4′-bipyridin-5-yl)methyl)-2,7-naphthyridin-1-amine;
N-((3-fluoro-2′-(trifluoromethyl)-2,4′-bipyridin-5-yl)methyl)-6-(2-methylpyridin-4-yl)-2,7-naphthyridin-1-amine;
N-(3-fluoro-4-(2-methylpyridin-4-yl))benzyl)-6-(2-methylpyridin-4-yl)-2,7-naphthyridin-1-amine;
N-((2′-fluoro-2,4′-bipyridin-5-yl)methyl)-6-(2-methylpyridin-4-yl)-2,7-naphthyridin-1-amine;
4-(5-(((6-(2-methylpyridin-4-yl)-2,7-naphthyridin-1-yl)amino)methyl)pyridine-2-yl)thiomorpholine 1,1-dioxide;
6-(2-methylpyridin-4-yl)-N-(4-(pyridazin-4-yl)benzyl)-2,7-naphthyridin-1-amine;
N-(4-(2-methylpyridin-4-yl)benzyl)-6-(pyrazin-2-yl)-2,7-naphthyridin-1-amine;
N-(4-(2-methylpyridin-4-yl)benzyl)-6-(pyridazin-4-yl)-2,7-naphthyridin-1-amine;
N-(4-(2-methylpyridin-4-yl)benzyl)-8-morpholino-2,7-naphthyridin-1-amine;
6-(4-methylpiperazin-1-yl)-N-(4-(2-methylpyridin-4-yl)benzyl)-2,7-naphthyridin-1-amine;
4-(8-((4-(2-methylpyridin-4-yl)benzyl)amino)-2,7-naphthyridin-3-yl)thiomorpholine 1,1-dioxide;
N-(3-fluoro-4-(2-fluoropyridin-4-yl)benzyl)-6-(3-fluorophenyl)-2,7-naphthyridin-1-amine;
N-(3-fluoro-4-(2-methylpyridin-4-yl)benzyl)-6-(3-fluorophenyl)-2,7-naphthyridin-1-amine;
N-((3-fluoro-2′-(trifluoromethyl)-2,4′-bipyridin-5-yl)methyl)-6-(3-fluorophenyl)-2,7-naphthyridin-1-amine;
N-((2′-fluoro-2,4′-bipyridin-5-yl)methyl)-6-(3-fluorophenyl)-2,7-naphthyridin-1-amine;
6-(3-fluorophenyl)-N-(3-methyl-4-(2-methylpyridin-4-yl)benzyl)-2,7-naphthyridin-1-amine;
4-(5-(((6-(3-fluorophenyl)-2,7-naphthyridin-1-yl)amino)methyl)pyridine-2-yl)thiomorpholine 1,1-dioxide;
N-(4-chlorobenzyl)-6-(2-methylpyridin-4-yl)-2,7-naphthyridin-1-amine;
N-(4-methylbenzyl)-6-(2-methylpyridin-4-yl)-2,7-naphthyridin-1-amine;
6-(2-methylpyridin-4-yl)-N-(pyridin-3-ylmethyl)-2,7-naphthyridin-1-amine;
N-benzyl-2-(3-fluorophenyl)-1,6-naphthyridin-5-amine;
2-(3-fluorophenyl)-N-((2′-methyl-2,4′-bipyridin-5-yl)methyl)-1,6-naphthyridin-5-amine;
N-((2′-methyl-2,4′-bipyridin-5-yl)methyl)-2-(2-methylpyridin-4-yl)-1,6-naphthyridin-5-amine;
N-((6-(3-fluorophenyl)pyridin-3-yl)methyl)-2-(2-methylpyridin-4-yl)-1,6-naphthyridin-5-amine;
N-(4-(2-fluoropyridin-4-yl)benzyl)-2-(2-methylpyridin-4-yl)-1,6-naphthyridin-5-amine;
2-(2-methylpyridin-4-yl)-N-(4-(2-(trifluoromethyl)pyridin-4-yl)benzyl)-1,6-naphthyridin-5-amine;
N-((2′,3-dimethyl-2,4′-bipyridin-5-yl)methyl)-2-(2-methylpyridin-4-yl)-1,6-naphthyridin-5-amine;
N-(biphenyl-4-ylmethyl)-6-(3-fluorophenyl)isoquinolin-1-amine;
N-((2-fluorobiphenyl-4-yl)methyl)-6-(3-fluorophenyl)isoquinolin-1-amine;
N-((2′-methyl-2,4′-bipyridin-5-yl)methyl)-6-phenylisoquinolin-1-amine;
6-(3-chlorophenyl)-N-((2′-methyl-2,4′-bipyridin-5-yl)methyl)isoquinolin-1-amine;
N-(4-(2-methylpyridin-4-yl)benzyl)-6-phenylisoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-4-yl)isoquinolin-1-amine;
6-(6-methylpyridin-3-yl)-N-(4-(2-methylpyridin-4-yl)benzyl)isoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-3-yl)isoquinolin-1-amine;
N-(4-(2-methylpyridin-4-yl)benzyl)-6-(pyrazin-2-yl)isoquinolin-1-amine;
N-(4-(2-methylpyridin-4-yl)benzyl)-6-(pyridazin-4-yl)isoquinolin-1-amine;
N-((2′-methyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyridin-2-yl)isoquinolin-1-amine;
N-((2′,3-dimethyl-2,4′-bipyridin-5-yl)methyl)-6-(3-fluorophenyl)isoquinolin-1-amine;
N-((2′,3-dimethyl-2,4′-bipyridin-5-yl)methy-6-(5-methylpyridin-3-yl)isoquinolin-1-amine;
N-((2′-methyl-2,4′-bipyridin-5-yl)methyl)-2-phenylpyrido[4,3-b]pyrazin-5-amine;
2-(3-fluorophenyl)-N-(4-(2-methylpyridin-4-yl)benzyl)pyrido[4,3-b]pyrazin-5-amine;
2-(3-fluorophenyl)-N-((2′-methyl-2,4′-bipyridin-5-yl)methyl)pyrido[4,3-b]pyrazin-5-amine;
2-(3-fluorophenyl)-N-(3-methyl-4-(2-methylpyridin-4-yl)benzyl)pyrido[4,3-b]pyrazin-5-amine;
N-(3-fluoro-4-(2-methylpyridin-4-yl)benzyl)-2-(3-fluorophenyl)pyrido[4,3-b]pyrazin-5-amine;
2-(2-methylpyridin-4-yl)-N-(4-(2-methylpyridin-4-yl)benzyl)pyrido[4,3-b]pyrazin-5-amine;
N-((2′-methyl-2,4′-bipyridin-5-yl)methyl)-2-(2-methylpyridin-4-yl)pyrido[4,3-b]pyrazin-5-amine;
N-(3-methyl-4-(2-methylpyridin-4-yl)benzyl)-2-(2-methylpyridin-4-yl)pyrido[4,3-b]pyrazin-5-amine;
N-(3-fluoro-4-(2-methylpyridin-4-yl)benzyl)-2-(2-methylpyridin-4-yl)pyrido[4,3-b]pyrazin-5-amine;
N-((2′,3-dimethyl-2,4′-bipyridin-5-yl)methyl)-6-(pyrazin-2-yl)-2,7-naphthyridin-1-amine;
6-(2-methylmorpholino)-N-(4-(2-methylpyridin-4-yl)benzyl)-2,7-naphthyridin-1-amine;
(S)-6-(2-methylmorpholino)-N-(4-(2-methylpyridin-4-yl)benzyl)-2,7-naphthyridin-1-amine;
(R)-6-(2-methylmorpholino)-N-(4-(2-methylpyridin-4-yl)benzyl)-2,7-naphthyridin-1-amine;
1-(4-(8-(4-(2-methylpyridin-4-yl)benzylamino)-2,7-naphthyridin-3-yl) piperazin-1-yl)ethanone;
6-(1H-imidazol-1-yl)-N-(4-(2-methylpyridin-4-yl)benzyl)-2,7-naphthyridin-1-amine;
6-(4-methyl-1H-imidazol-1-yl)-N-(4-(2-methylpyridin-4-yl)benzyl)-2,7-naphthyridin-1-amine;
N-(4-(2-methylpyridin-4-yl)benzyl)-6-(1H-tetrazol-5-yl)-2,7-naphthyridin-1-amine;
6-(5-methyl-1,3,4-oxadiazol-2-yl)-N-(4-(2-methylpyridin-4-yl)benzyl)-2,7-naphthyridin-1-amine;
6-(1-methyl-1H-pyrazol-3-yl)-N-(4-(2-methylpyridin-4-yl)benzyl)-2,7-naphthyridin-1-amine;
N-(4-(2-methylpyridin-4-yl)benzyl)-6-(thiazol-5-yl)-2,7-naphthyridin-1-amine;
N-(4-(2-methylpyridin-4-yl)benzyl)-6-(oxazol-5-yl)-2,7-naphthyridin-1-amine;
N-((2′,3-dimethyl-2,4′-bipyridin-5-yl)methyl)-6-(5-methylpyridin-3-yl)-2,7-naphthyridin-1-amine;
N-((2′,3-dimethyl-2,4′-bipyridin-5-yl)methyl)-6-(2-methylpyridin-4-yl)-2,7-naphthyridin-1-amine;
N-((3-fluoro-2′-methyl-2,4′-bipyridin-5-yl)methyl)-6-(2-methylpyridin-4-yl)-2,7-naphthyridin-1-amine;
N-((2′,3-dimethyl-2,4′-bipyridin-5-yl)methyl)-6-(5-fluoropyridin-3-yl)-2,7-naphthyridin-1-amine;
N-(3-methyl-4-(2-methylpyridin-4-yl)benzyl)-6-(pyrazin-2-yl)-2,7-naphthyridin-1-amine;
N-(3-fluoro-4-(2-methylpyridin-4-yl)benzyl)-6-(pyrazin-2-yl)-2,7-naphthyridin-1-amine;
methyl 4-(8-(4-(2-methylpyridin-4-yl)benzylamino)-2,7-naphthyridin-3-yl)piperazine-1-carboxylate;
4-(8-(4-(2-methylpyridin-4-yl)benzylamino)-2,7-naphthyridin-3-yl)piperazin-2-one;
2-(4-(8-(4-(2-methylpyridin-4-yl)benzylamino)-2,7-naphthyridin-3-yl)piperazin-1-yl)acetonitrile;
2-methyl-4-(4-((6-(2-methylpyridin-4-yl)-2,7-naphthyridin-1-ylamino)methyl)phenylpyridine 1-oxide;
6-(2-chloropyridin-4-yl)-N-((2′,3-dimethyl-2,4′-bipyridin-5-yl)methyl)-2,7-naphthyridin-1-amine;
6-(2-chloropyridin-4-yl)-N-(4-(2-methylpyridin-4-yl)benzyl)-2,7-naphthyridin-1-amine;
2-(2-methylpyridin-4-yl)-5-((6-(2-methylpyridin-4-yl)-2,7-naphthyridin-1-ylamino)methyl)benzonitrile;
N-(3-methoxy-4-(2-methylpyridin-4-yl)benzyl)-6-(2-methylpyridin-4-yl)-2,7-naphthyridin-1-amine;
N-((3-chloro-2′-methyl-2,4′-bipyridin-5-yl)methyl)-6-(2-methylpyridin-4-yl)-2,7-naphthyridin-1-amine;
2′-methyl-5-((6-(2-methylpyridin-4-yl)-2,7-naphthyridin-1-ylamino)methyl)-2,4′-bipyridine-3-carbonitrile; and N-(4-(2-(difluoromethyl)pyridin-4-yl)benzyl)-6-(2-methylpyridin-4-yl)-2,7-naphthyridin-1-amine.

In some embodiments, the Porcupine antagonist comprises a compound of the following Formula (II):

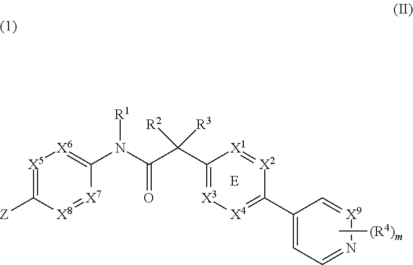

or a physiologically acceptable salt thereof, wherein:
X¹, X², X³ and X⁴ is selected from N and CR⁷;
one of X⁵, X₆, X⁷ and X⁸ is N and the others are CH;
X⁹ is selected from N and CH;
Z is selected from phenyl, pyrazinyl, pyridinyl, pyridazinyl and piperazinyl;
wherein each phenyl, pyrazinyl, pyridinyl, pyridazinyl or piperazinyl of Z is optionally substituted with an R⁶ group;
R¹, R² and R³ are hydrogen;
m is 1;
R₄ is selected from hydrogen, halo, difluoromethyl, trifluoromethyl and methyl;
R⁶ is selected from hydrogen, halo and —C(O)Rⁱ⁰; wherein R¹⁰ is methyl; and
R⁷ is selected from hydrogen, halo, cyano, methyl and trifluoromethyl.

In some embodiments, the compound is selected from the group of:
N-[5-(3-fluorophenyl)pyridin-2-yl]-2-[5-methyl-6-(pyridazin-4-yl)pyridin-3-yl]acetamide;
2-[5-methyl-6-(2-methylpyridin-4-yl)pyridin-3-yl]-N-[5-(pyrazin-2-yl)pyridin-2-yl]acetamide (LGK974);
N-(2,3′-bipyridin-6′-yl)-2-(2′,3-dimethyl-2,4′-bipyridin-5-yl)acetamide;
N-(5-(4-acetylpiperazin-1-yl)pyridin-2-yl)-2-(2′-methyl-3-(trifluoromethyl)-2,4′-bipyridin-5-yl)acetamide;
N-(5-(4-acetylpiperazin-1-yl)pyridin-2-yl)-2-(2′-fluoro-3-methyl-2,4′-bipyridin-5-yl)acetamide; and
2-(2′-fluoro-3-methyl-2,4′-bipyridin-5-yl)-N-(5-(pyrazin-2-yl)pyridin-2-yl)acetamide; and a pharmaceutically acceptable salt thereof.

In some embodiments, the compound is 2-[5-methyl-6-(2-methylpyridin-4-yl)pyridin-3-yl]-N-[5-(pyrazin-2-yl)pyridin-2-yl]acetamide.

In some embodiments, the therapeutically effective amount of the compound is about 0.01 to 20 mg/kg per body weight at daily dosages.

In some embodiments, the therapeutically effective amount of the compound from about 0.5 mg to about 1000 mg for humans.

In some embodiments, wherein the cancer is colorectal cancer, gastric cancer, liver cancer, esophageal cancer, intestinal cancer, bile duct cancer, pancreatic cancer, endometrial cancer, or prostate cancer.

In another aspect, the present invention provides a method for determining whether a subject with cancer should be treated with a composition that inhibits Wnt activity, the method comprising: (a) isolating a biological sample from the subject; (b) performing an assay on the biological sample to identify the presence or absence of an R-spondin fusion; and (c) determining that the subject should be treated with a composition comprising a therapeutically effective amount of an antagonist of Porcupine if the biological sample contains an R-spondin fusion.

In some embodiments, the R-spondin fusion comprising: (1) a PTPRKe1-Rspo3e2 fusion; (2) a PTPRKe7-Rspo3e2 fusion; (3) an EIF3Ee1-Rspo2e2 fusion; or (4) an EIF3Ee1-Rspo2e3 fusion.

In some embodiments, the R-spondin fusion comprising: (1) an EMC2e1-Rspo2e2 fusion; (2) a PVT1-Rspo2e2 fusion; (3) a PVT1-Rspo2e3 fusion; (4) an HNF4G-Rspo2e2 fusion; (5) a PTPRKe13-Rspo3e2 fusion; or (6) a PTPRKe6X-Rspo3e2 fusion.

In some embodiments, the subject is determined to have R-spondin mRNA expression level that is higher than the R-spondin mRNA expression level in a control subject that has been determined that does not have a R-spondin fusion.

In some embodiments, the -Rspondin fusion comprises a junction sequence of any one of SEQ ID NO.:58, SEQ ID.:59, SEQ ID NO.:62, or SEQ ID NO.: 63.

In some embodiments, the EMC2e1-Rspo2e2 fusion comprises a junction sequence of SEQ ID NO.:64.

In some embodiments, the PVT1-Rspo2e2 fusion comprises a junction sequence of SEQ ID NO.:65.

In some embodiments, the PVT1-Rspo2e3 fusion comprises a junction sequence of SEQ ID NO.:66.

In some embodiments, the HNF4G-Rspo2e2 fusion comprises a junction sequence of SEQ ID NO.:67.

In some embodiments, the PTPRKe13-Rspo3e2 fusion comprises a junction sequence of SEQ ID NO.:61.

In some embodiments, the PTPRKe6X-Rspo3e2 fusion comprises a junction sequence of SEQ ID NO.:60.

In some embodiments, the Rspondin is Rspo2 or Rsp3, and the fusion gene is overexpressed in comparison to the Rspondin that is not fused to another gene.

In some embodiments, the Porcupine antagonist comprise a compound of Formula (I):

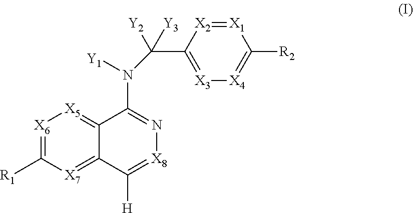

or a physiologically acceptable salt thereof, wherein
X₁, X₂, X₃, X₄, X₅, X₆, X₇, X₈ are independently CR₄ or N;
Y₁ is hydrogen or CR₄; Y₂, Y₃ are independent hydrogen, halo or CR₃;
R₁ is morpholinyl, piperazinyl, quinolinyl,

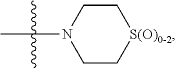

aryl, C₁₋₆ heterocycle, 5 or 6 membered heteroaryl containing 1-2 heteroatoms selected from N, O and S;
R₂ is hydrogen, halo, morpholinyl, piperazinyl, quinolinyl,

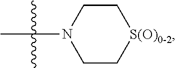

aryl, C₁₋₆ heterocycle, 5 or 6 membered heteroaryl containing 1-2 heteroatoms selected from N, O and S; Ra is hydrogen, halo, cyano, C₁₋₆ alkyl, C₁₋₆ alkoxy optionally substituted with halo, amino, hydroxyl, alkoxy or cyano;
R₄ is hydrogen, halo, C₁₋₆ alkoxy, —S(O)₂R₅, —C(O)OR₅, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano.

In some embodiments, the 5 or 6 membered heteroaryl is selected from:

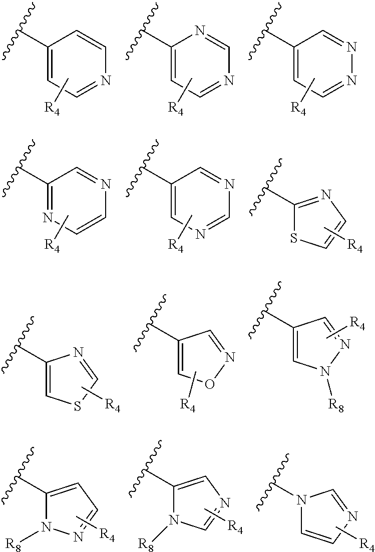

wherein,
R₄ is hydrogen, halo, C₁₋₆ alkoxy, —S(O)₂R₅, —C(O)OR₅, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano; and R₈ is hydrogen or C₁₋₆ alkyl.

In some embodiments, R₁ and R₂ is independently substituted with 1 or 2 R₄ groups.

In some embodiments, the compound is selected from 6-(2-methylpyridin-4-yl)-N-(4-(2-methylpyridin-4-yl)benzyl)-2,7-naphthyridin-1-amine;
N-(3-methyl-4-(2-methylpyridin-4-yl)benzyl)-6-(2-methylpyridin-4-yl)-2,7-naphthyridin-1-amine;
6-(3-fluorophenyl)-N-((2′-methyl-2,4′-bipyridin-5-yl)methyl)isoquinolin-1-amine;
2-(2-methylpyridin-4-yl)-N-(4-(2-methylpyridin-4-yl)benzyl)-1,6-naphthyridin-5-amine;
N-(4-(2-methylpyridin-4-yl)benzyl)-2-phenylpyrido[4,3-b]pyrazin-5-amine;
N-(4-(2-methylpyridin-4-yl)benzyl)-6-(pyridin-4-yl)-2,7-naphthyridin-1-amine;
N-(4-(2-methylpyridin-4-yl)benzyl)-6-phenyl-2,7-naphthyridin-1-amine;
6-(3-chlorophenyl)-N-(4-(2-methylpyridin-4-yl)benzyl)-2,7-naphthyridin-1-amine;
6-(3-fluorophenyl)-N-(4-(2-methylpyridin-4-yl)benzyl)-2,7-naphthyridin-1-amine;
6-(3-fluorophenyl)-N-((2′-methyl-2,4′-bipyridin-5-yl)methyl)-2,7-naphthyridin-1-amine;
6-(3-fluorophenyl)-N-(4-(2-(trifluoromethyl)pyridin-4-yl)benzyl)-2,7-naphthyridin-1-amine;
N-((2′,3-dimethyl-2,4′-bipyridin-5-yl)methy-6-(3-fluorophenyl)-2,7-naphthyridin-1-amine;
N-(4-(2-methylpyridin-4-yl)benzyl)-6-(pyrimidin-5-yl)-2,7-naphthyridin-1-amine;
6-(5-methylpyridin-3-yl)-N-(4-(2-methylpyridin-4-yl)benzyl)-2,7-naphthyridin-1-amine;
6-(6-methylpyridin-3-yl)-N-(4-(2-methylpyridin-4-yl)benzyl)-2,7-naphthyridin-1-amine;
3-(8-(4-(2-methylpyridin-4-yl)benzylamino)-2,7-naphthyridin-3-yl)benzonitrile;
4-(8-(4-(2-methylpyridin-4-yl)benzylamino)-2,7-naphthyridin-3-yl)benzonitrile;
6-(4-fluorophenyl)-N-(4-(2-methylpyridin-4-yl)benzyl)-2,7-naphthyridin-1-amine;
N-(4-(2-methylpyridin-4-yl)benzyl)-8-m-tolyl-2,7-naphthyridin-1-amine;
N-(4-(2-methylpyridin-4-yl)benzyl)-2,7-naphthyridin-1-amine;
N-(4-(2-methylpyridin-4-yl)benzyl)-6-(pyridin-2-yl)-2,7-naphthyridin-1-amine;
6-(2-fluoropyridin-4-yl)-N-(4-(2-methylpyridin-4-yl)benzyl)-2,7-naphthyridin-1-amine;
6-(2-fluorophenyl)-N-(4-(2-methylpyridin-4-yl)benzyl)-2,7-naphthyridin-1-amine;
N-(4-(2-methylpyridin-4-yl)benzyl)-6-(pyridin-3-yl)-2,7-naphthyridin-1-amine;
N-(biphenyl)-4-ylmethyl)-6-(2-methylpyridin-4-yl)-2,7-naphthyridin-1-amine;
6-(2-methylpyridin-4-yl)-N-((5-phenylpyridin-2-yl)methy-2,7-naphthyridin-1-amine;
6-(3-fluorophenyl)-N-((2′-(trifluoromethyl)-2,4′-bipyridin-5-yl)methyl)-2,7-naphthyridin-1-amine;
N-(3-fluoro-4-(2-fluoropyridin-4-yl)benzyl)-6-(2-methylpyridin-4-yl)-2,7-naphthyridin-1-amine;
6-(2-methylpyridin-4-yl)-N-((2′-(trifluoromethyl)-2,4′-bipyridin-5-yl)methyl)-2,7-naphthyridin-1-amine;
N-((3-fluoro-2′-(trifluoromethyl)-2,4′-bipyridin-5-yl)methyl)-6-(2-methylpyridin-4-yl)-2,7-naphthyridin-1-amine;
N-(3-fluoro-4-(2-methylpyridin-4-yl))benzyl)-6-(2-methylpyridin-4-yl)-2,7-naphthyridin-1-amine;
N-((2′-fluoro-2,4′-bipyridin-5-yl)methyl)-6-(2-methylpyridin-4-yl)-2,7-naphthyridin-1-amine;
4-(5-(((6-(2-methylpyridin-4-yl)-2,7-naphthyridin-1-yl)amino)methyl)pyridine-2-yl)thiomorpholine 1,1-dioxide;
6-(2-methylpyridin-4-yl)-N-(4-(pyridazin-4-yl)benzyl)-2,7-naphthyridin-1-amine;
N-(4-(2-methylpyridin-4-yl)benzyl)-6-(pyrazin-2-yl)-2,7-naphthyridin-1-amine;
N-(4-(2-methylpyridin-4-yl)benzyl)-6-(pyridazin-4-yl)-2,7-naphthyridin-1-amine;
N-(4-(2-methylpyridin-4-yl)benzyl)-8-morpholino-2,7-naphthyridin-1-amine;
6-(4-methylpiperazin-1-yl)-N-(4-(2-methylpyridin-4-yl)benzyl)-2,7-naphthyridin-1-amine;
4-(8-((4-(2-methylpyridin-4-yl)benzyl)amino)-2,7-naphthyridin-3-yl)thiomorpholine 1,1-dioxide;
N-(3-fluoro-4-(2-fluoropyridin-4-yl)benzyl)-6-(3-fluorophenyl)-2,7-naphthyridin-1-amine;
N-(3-fluoro-4-(2-methylpyridin-4-yl)benzyl)-6-(3-fluorophenyl)-2,7-naphthyridin-1-amine;
N-((3-fluoro-2′-(trifluoromethyl)-2,4′-bipyridin-5-yl)methyl)-6-(3-fluorophenyl)-2,7-naphthyridin-1-amine;
N-((2′-fluoro-2,4′-bipyridin-5-yl)methyl)-6-(3-fluorophenyl)-2,7-naphthyridin-1-amine;
6-(3-fluorophenyl)-N-(3-methyl-4-(2-methylpyridin-4-yl)benzyl)-2,7-naphthyridin-1-amine;
4-(5-(((6-(3-fluorophenyl)-2,7-naphthyridin-1-yl)amino)methyl)pyridine-2-yl)thiomorpholine 1,1-dioxide;
N-(4-chlorobenzyl)-6-(2-methylpyridin-4-yl)-2,7-naphthyridin-1-amine;
N-(4-methylbenzyl)-6-(2-methylpyridin-4-yl)-2,7-naphthyridin-1-amine;
6-(2-methylpyridin-4-yl)-N-(pyridin-3-ylmethyl)-2,7-naphthyridin-1-amine;
N-benzyl-2-(3-fluorophenyl)-1,6-naphthyridin-5-amine;
2-(3-fluorophenyl)-N-((2′-methyl-2,4′-bipyridin-5-yl)methyl)-1,6-naphthyridin-5-amine;
N-((2′-methyl-2,4′-bipyridin-5-yl)methyl)-2-(2-methylpyridin-4-yl)-1,6-naphthyridin-5-amine;
N-((6-(3-fluorophenyl)pyridin-3-yl)methyl)-2-(2-methylpyridin-4-yl)-1,6-naphthyridin-5-amine;
N-(4-(2-fluoropyridin-4-yl)benzyl)-2-(2-methylpyridin-4-yl)-1,6-naphthyridin-5-amine;
2-(2-methylpyridin-4-yl)-N-(4-(2-(trifluoromethyl)pyridin-4-yl)benzyl)-1,6-naphthyridin-5-amine;
N-((2′,3-dimethyl-2,4′-bipyridin-5-yl)methyl)-2-(2-methylpyridin-4-yl)-1,6-naphthyridin-5-amine;
N-(biphenyl-4-ylmethyl)-6-(3-fluorophenyl)isoquinolin-1-amine;
N-((2-fluorobiphenyl-4-yl)methyl)-6-(3-fluorophenyl)isoquinolin-1-amine;
N-((2′-methyl-2,4′-bipyridin-5-yl)methyl)-6-phenylisoquinolin-1-amine;
6-(3-chlorophenyl)-N-((2′-methyl-2,4′-bipyridin-5-yl)methyl)isoquinolin-1-amine;
N-(4-(2-methylpyridin-4-yl)benzyl)-6-phenylisoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-4-yl)isoquinolin-1-amine;
6-(6-methylpyridin-3-yl)-N-(4-(2-methylpyridin-4-yl)benzyl)isoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-3-yl)isoquinolin-1-amine;
N-(4-(2-methylpyridin-4-yl)benzyl)-6-(pyrazin-2-yl)isoquinolin-1-amine;
N-(4-(2-methylpyridin-4-yl)benzyl)-6-(pyridazin-4-yl)isoquinolin-1-amine;
N-((2′-methyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyridin-2-yl)isoquinolin-1-amine;
N-((2′,3-dimethyl-2,4′-bipyridin-5-yl)methyl)-6-(3-fluorophenyl)isoquinolin-1-amine;
N-((2′,3-dimethyl-2,4′-bipyridin-5-yl)methy-6-(5-methylpyridin-3-yl)isoquinolin-1-amine;
N-((2′-methyl-2,4′-bipyridin-5-yl)methyl)-2-phenylpyrido[4,3-b]pyrazin-5-amine;
2-(3-fluorophenyl)-N-(4-(2-methylpyridin-4-yl)benzyl)pyrido[4,3-b]pyrazin-5-amine;
2-(3-fluorophenyl)-N-((2′-methyl-2,4′-bipyridin-5-yl)methyl)pyrido[4,3-b]pyrazin-5-amine;
2-(3-fluorophenyl)-N-(3-methyl-4-(2-methylpyridin-4-yl)benzyl)pyrido[4,3-b]pyrazin-5-amine;
N-(3-fluoro-4-(2-methylpyridin-4-yl)benzyl)-2-(3-fluorophenyl)pyrido[4,3-b]pyrazin-5-amine;
2-(2-methylpyridin-4-yl)-N-(4-(2-methylpyridin-4-yl)benzyl)pyrido[4,3-b]pyrazin-5-amine;
N-((2′-methyl-2,4′-bipyridin-5-yl)methyl)-2-(2-methylpyridin-4-yl)pyrido[4,3-b]pyrazin-5-amine;
N-(3-methyl-4-(2-methylpyridin-4-yl)benzyl)-2-(2-methylpyridin-4-yl)pyrido[4,3-b]pyrazin-5-amine;
N-(3-fluoro-4-(2-methylpyridin-4-yl)benzyl)-2-(2-methylpyridin-4-yl)pyrido[4,3-b]pyrazin-5-amine;
N-((2′,3-dimethyl-2,4′-bipyridin-5-yl)methyl)-6-(pyrazin-2-yl)-2,7-naphthyridin-1-amine;
6-(2-methylmorpholino)-N-(4-(2-methylpyridin-4-yl)benzyl)-2,7-naphthyridin-1-amine;
(S)-6-(2-methylmorpholino)-N-(4-(2-methylpyridin-4-yl)benzyl)-2,7-naphthyridin-1-amine;
(R)-6-(2-methylmorpholino)-N-(4-(2-methylpyridin-4-yl)benzyl)-2,7-naphthyridin-1-amine;
1-(4-(8-(4-(2-methylpyridin-4-yl)benzylamino)-2,7-naphthyridin-3-yl)piperazin-1-yl)ethanone;
6-(1H-imidazol-1-yl)-N-(4-(2-methylpyridin-4-yl)benzyl)-2,7-naphthyridin-1-amine;
6-(4-methyl)-1H-imidazol-1-yl)-N-(4-(2-methylpyridin-4-yl)benzyl)-2,7-naphthyridin-1-amine;
N-(4-(2-methylpyridin-4-yl)benzyl)-6-(1H-tetrazol-5-yl)-2,7-naphthyridin-1-amine;
6-(5-methyl-1,3,4-oxadiazol-2-yl)-N-(4-(2-methylpyridin-4-yl)benzyl)-2,7-naphthyridin-1-amine;
6-(1-methyl-1H-pyrazol-3-yl)-N-(4-(2-methylpyridin-4-yl)benzyl)-2,7-naphthyridin-1-amine;
N-(4-(2-methylpyridin-4-yl)benzyl)-6-(thiazol-5-yl)-2,7-naphthyridin-1-amine;
N-(4-(2-methylpyridin-4-yl)benzyl)-6-(oxazol-5-yl)-2,7-naphthyridin-1-amine;
N-((2′,3-dimethyl-2,4′-bipyridin-5-yl)methyl)-6-(5-methylpyridin-3-yl)-2,7-naphthyridin-1-amine;
N-((2′,3-dimethyl-2,4′-bipyridin-5-yl)methyl)-6-(2-methylpyridin-4-yl)-2,7-naphthyridin-1-amine;
N-((3-fluoro-2′-methyl-2,4′-bipyridin-5-ylmethyl)-6-(2-methylpyridin-4-yl)-2,7-naphthyridin-1-amine;
N-((2′,3-dimethyl-2,4′-bipyridin-5-yl)methyl)-6-(5-fluoropyridin-3-yl)-2,7-naphthyridin-1-amine;
N-(3-methyl-4-(2-methylpyridin-4-yl)benzyl)-6-(pyrazin-2-yl)-2,7-naphthyridin-1-amine;
N-(3-fluoro-4-(2-methylpyridin-4-yl)benzyl)-6-(pyrazin-2-yl)-2,7-naphthyridin-1-amine; methyl 4-(8-(4-(2-methylpyridin-4-yl)benzylamino)-2,7-naphthyridin-3-yl)piperazine-1-carboxylate;
4-(8-(4-(2-methylpyridin-4-yl)benzylamino)-2,7-naphthyridin-3-yl)piperazin-2-one;
2-(4-(8-(4-(2-methylpyridin-4-yl)benzylamino)-2,7-naphthyridin-3-yl)piperazin-1-yl)acetonitrile;
2-methyl-4-(4-((6-(2-methylpyridin-4-yl)-2,7-naphthyridin-1-ylamino)methylphenylpyridine 1-oxide;
6-(2-chloropyridin-4-yl)-N-((2′,3-dimethyl-2,4′-bipyridin-5-yl)methyl)-2,7-naphthyridin-1-amine;
6-(2-chloropyridin-4-yl)-N-(4-(2-methylpyridin-4-yl)benzyl)-2,7-naphthyridin-1-amine;
2-(2-methylpyridin-4-yl)-5-((6-(2-methylpyridin-4-yl)-2,7-naphthyridin-1-ylamino)methyl)benzonitrile;
N-(3-methoxy-4-(2-methylpyridin-4-yl)benzyl)-6-(2-methylpyridin-4-yl)-2,7-naphthyridin-1-amine;
N-((3-chloro-2′-methyl-2,4′-bipyridin-5-yl)methyl)-6-(2-methylpyridin-4-yl)-2,7-naphthyridin-1-amine;
2′-methyl-5-((6-(2-methylpyridin-4-yl)-2,7-naphthyridin-1-ylamino)methyl)-2,4′-bipyridine-3-carbonitrile; and N-(4-(2-(difluoromethyl)pyridin-4-yl)benzyl)-6-(2-methylpyridin-4-yl)-2,7-naphthyridin-1-amine.

In some embodiments, wherein the Porcupine antagonist comprises a compound of Formula (II):

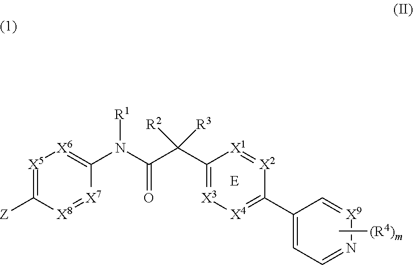

or a physiologically acceptable salt thereof, wherein:
X¹, X², X³ and X⁴ is selected from N and CR⁷;
one of X⁵, X₆, X⁷ and X⁸ is N and the others are CH;
X⁹ is selected from N and CH;
Z is selected from phenyl, pyrazinyl, pyridinyl, pyridazinyl and piperazinyl;
wherein each phenyl, pyrazinyl, pyridinyl, pyridazinyl or piperazinyl of Z is optionally substituted with an R⁶ group;
R¹, R² and R³ are hydrogen;
m is 1;
R₄ is selected from hydrogen, halo, difluoromethyl, trifluoromethyl and methyl;
R⁶ is selected from hydrogen, halo and —C(O)Rⁱ⁰: wherein R¹⁰ is methyl; and
R⁷ is selected from hydrogen, halo, cyano, methyl and trifluoromethyl.

In some embodiments, the compound is selected from the group of:
N-[5-(3-fluorophenyl)pyridin-2-yl]-2-[5-methyl-6-(pyridazin-4-yl)pyridin-3-yl]acetamide;
2-[5-methyl-6-(2-methylpyridin-4-yl)pyridin-3-yl]-N-[5-(pyrazin-2-yl)pyridin-2-yl]acetamide (LGK974);
N-(2,3′-bipyridin-6′-yl)-2-(2′,3-dimethyl-2,4′-bipyridin-5-yl)acetamide;
N-(5-(4-acetylpiperazin-1-yl)pyridin-2-yl)-2-(2′-methyl-3-(trifluoromethyl)-2,4′-bipyridin-5-yl)acetamide;
N-(5-(4-acetylpiperazin-1-yl)pyridin-2-yl)-2-(2′-fluoro-3-methyl-2,4′-bipyridin-5-yl)acetamide; and
2-(2′-fluoro-3-methyl-2,4′-bipyridin-5-yl)-N-(5-(pyrazin-2-yl)pyridin-2-yl)acetamide; or a pharmaceutically acceptable salt thereof.

In some embodiments, the compound is 2-[5-methyl-6-(2-methylpyridin-4-yl)pyridin-3-yl]-N-[5-(pyrazin-2-yl)pyridin-2-yl]acetamide.

In some embodiments, the cancer is colorectal cancer, gastric cancer, liver cancer, esophageal cancer, intestinal cancer, bile duct cancer, pancreatic cancer, endometrial cancer, or prostate cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts Rspo2 Nanostring nCounter decision making chart.

FIG. 2 depicts Rspo3 Nanostring nCounter decision making chart.

FIG. 3 depicts validation of Nanostring nCounter genotyping assay with characterized tumor tissues (Table 7). The shade highlights an expected positive signal in the characterized sample. Signal count of Rspo2 exon1 in L440 samples is close to the count in other exons, indicating the expression of wild type Rspo2 transcripts instead of Rspo2 fusion in L440 tumor sample.

FIG. 4 depicts the quantitation of Rspo2 and Rspo3 transcripts by Nanostring nCounter assay in tumor samples with and without Rspo2 fusion or Rspo3 fusion genes.

FIGS. 5A and 5B depicts the sequences of various Rspo2 (Table 8) and Rspo3 (Table 9) gene fusions.

FIGS. 6A-6C depict anti-tumor effect of CGX1321 in the tumor models carrying RSPO3 Fusion Genes. FIG. 6A: Dose response of CGX1321 on CRC011 PDX model of colorectal tumor with PTPRKe-Rspo3 gene fusion that fuses exon1 of PTPRK to exon2 of Rspo3. Upon tumors reaching ˜150 mm³ size, various doses of CGX1321 were administered as indicated for 28 days. Tumor sizes were measured twice a week in each group (n=8 animals/group). FIG. 6B: CRC141 colorectal tumor PDX model with type 2 PTPRK-Rspo3 gene fusion that fuses exon7 of PTPRK to exon2 of Rspo3. Upon tumors reaching ˜150 mm³ size, CGX1321 was administered at 7.5 mg/kg QD orally for 21 days. Tumor sizes were measured twice a week (n=8 animals/group). FIG. 6C: CR2506 colorectal tumor PDX model with type3 PTPRK-Rspo3 gene fusion that fuses exon13 to Rspo3 exon2. Left: Tumor growth curve of xenograft tumor CR2506 model without treatment in 12 independent experiments as historical controls provided by the service provider. Right: Tumor growth of CR2506 model with the treatment of CGX1321 at 1 mg/kg QD orally for 28 days. Tumor sizes were measured twice a week in each group (n=4 animals/group).

FIGS. 7A-7C depict anti-tumor effect of CGX1321 in the tumor models carrying Rspo2 fusion genes. FIG. 7A: GA67 gastric tumor PDX model with EMC2e1-Rspo2e2 gene fusion that fuses exon1 of EMC2 to exon2 of Rspo2. Upon tumors reaching ˜100 mm³ size, CGX1321 was administered at 1 mg/kg QD orally for 28 days. Tumor sizes were measured twice a week (n=6 animals/group). FIG. 7B: CR3056 colorectal tumor PDX model with PVT1e1-Rspo2e2 gene fusion that fuses exon1 of PVT1 to exon2 of Rspo2. Upon tumors reaching ˜100 mm³ size, CGX1321 was administered at 1 mg/kg QD orally for 28 days. Tumor sizes were measured twice a week in each group (n=6 animals/group). FIG. 7C: GA3055 gastric tumor PDX model with HFN4G-Rspo2e2 gene fusion that fuses HFN4G 5′ end to Rspo2 exon2. (Left: Tumor growth without the treatment provided by the service provider. Right: Tumor growth with the treatment of CGX1321 at 1 mg/kg QD orally for 28 days. Tumor sizes were measured twice a week in each group (n=4 animals/group).

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events.

Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### I. Definitions and Abbreviations

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well-known and commonly employed in the art. Standard techniques, or modifications thereof, are used for chemical syntheses and chemical analyses.

As used herein, “WNT signaling pathway” or “WNT pathway” refers to the pathway by which binding of the WNT protein to cellular receptors results in changes of cell behavior. The WNT pathway involves a variety of proteins including Frizzled, Disheveled, Axin, APC, GSK3β, β-catenin, LEF/TCF transcription factors, and molecules involved in the synthesis and secretion of WNT proteins. Examples of proteins implicated in the secretion of functional WNTs include, but are not limited to wntless/evenness interrupted (Wls/Evi), porcupine (Porcn), and Vps35p. Wls/Evi is a 7 pass transmembrane protein which resides in the Golgi apparatus and is required for secretion of Wg (drosophila) MOM-2 (C. elegans) and Wnt3A. It contains a conserved structural motif whose structure and function are both unknown. Porcupine (Porcn) is a member of the membrane-bound O-acyltransferase (MBOAT) family of palmitolyl transferases. Fatty acid modification of Wnts is critical for their function. Wnts are palmitolylated on one or two highly conserved sites. Inhibitors of Porcn may therefore block all functional Wnt signaling. Vps35p is a subunit of a multiprotein complex called the retromer complex which is involved in intracellular protein trafficking. Vps35p functions in binding target proteins like WNTs for recruitment into vesicles.

An “Wnt inhibitor” as used herein reduces the activity of Wnt pathway. Wnt inhibitors are compounds which can inhibit the Wnt signaling pathways, and include the PORCN inhibitors. This inhibition may include, for example, inhibiting PORCN, and its palmitolylation of Wnt, or reducing the association between the Wnt pathway components including Frizzled and Disheveled. Preferably a Wnt inhibitor is a PORCN inhibitor.

The term “a method of inhibiting WNT pathway” refers to methods of inhibiting known biochemical events associated with production of functional WNT proteins or with cellular responses to WNT proteins. As discussed herein, small organic molecules may inhibit WNT response in accordance with this definition.

“WNT protein” is a protein binds to Frizzled and LRP5/6 co-receptors so as to activate canonical or non-canonical WNT signaling. Specific examples of WNT proteins include: WNT-1 (NM005430), WNT-2 (NM003391), WNT-2B/WNT-13 (NM004185), WNT-3 (NM030753), WNT3a (NM033131), WNT-4 (NM030761), WNT-5A (NM003392), WNT-5B (NM032642), WNT-6 (NM006522), WNT-7A (NM004625), WNT-7B (NM058238), WNT-8A (NM058244), WNT-8B (NM003393), WNT-9A/WNT-14) (NM003395), WNT-9B/WNT-15 (NM003396), WNT-10A (NM025216), WNT-10B (NM003394), WNT-11 (NM004626), WNT-16 (NM016087).

“WNT pathway disorder” is a condition or disease state with aberrant WNT signaling. In one aspect, the aberrant WNT signaling is a level of WNT signaling in a cell or tissue suspected of being diseased that exceeds the level of WNT signaling in a normal cell or tissue. In one specific aspect, a WNT-mediated disorder includes cancer or fibrosis.

The term “cancer” refers to the pathological condition in humans that is characterized by unregulated cell proliferation. Examples include but are not limited to: carcinoma, lymphoma, blastoma, and leukemia. More particular examples of cancers include but are not limited to: lung (small cell and non-small cell), breast, prostate, carcinoid, bladder, gastric, pancreatic, liver (hepatocellular), hepatoblastoma, colorectal, head and neck squamous cell carcinoma, esophageal, ovarian, cervical, endometrial, mesothelioma, melanoma, sarcoma, osteosarcoma, liposarcoma, thyroid, desmoids, chronic myelocytic leukemia (AML), and chronic myelocytic leukemia (CML).

The term “fibrosis” refers to the pathological condition in humans that is typically characterized by uncontrolled proliferation of fibroblast cells and tissue hardening. Specific examples include but not limited to: lung fibrosis (idiopathic pulmonary fibrosis and radiation-induced fibrosis), renal fibrosis and liver fibrosis including liver cirrhosis.

“Inhibiting” or “treating” or “treatment” refers to reduction, therapeutic treatment and prophylactic or preventative treatment, wherein the objective is to reduce or prevent the aimed pathologic disorder or condition. In one example, following administering of a WNT signaling inhibitor, a cancer patient may experience a reduction in tumor size. “Treatment” or “treating” includes (1) inhibiting a disease in a subject experiencing or displaying the pathology or symptoms of the disease, (2) ameliorating a disease in a subject that is experiencing or displaying the pathology or symptoms of the disease, and/or (3) affecting any measurable decrease in a disease in a subject or patient that is experiencing or displaying the pathology or symptoms of the disease. To the extent the WNT pathway inhibitor may prevent growth and/or kill cancer cells, it may be cytostatic and/or cytotoxic.

The term “therapeutically effective amount” refers to an amount of a WNT pathway inhibitor (e.g. a Porcupine antagonist) effective to “treat” a WNT pathway disorder in a subject or mammal. In the case of cancer, the therapeutically effective amount of the drug may either reduce the number of cancer cells, reduce the tumor size, inhibit cancer cell infiltration into peripheral organs, inhibit tumor metastasis, inhibit tumor growth to certain extent, and/or relieve one or more of the symptoms associated with the cancer to some extent.

Administration “in combination with” one or more further therapeutic agents includes simultaneous (concurrent) and consecutive administration in any order. As used herein, the term “pharmaceutical combination” refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

A“chemotherapeutic agent” is a chemical compound useful in the treatment of cancer. Examples are but not limited to: Gemcitabine, Irinotecan, Doxorubicin, 5-Fluorouracil, Cytosine arabinoside (“Ara-C”), Cyclophosphamide, Thiotepa, Busulfan, Cytoxin, TAXOL, Methotrexate, Cisplatin, Melphalan, Vinblastine and Carboplatin.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups, are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A“lower alkyl” or “lower alkylene” is a shorter chain alkyl or alkylene group, generally having eight or fewer carbon atoms.

The terms “alkoxy,” “alkylamino” and “alkytthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazoyl, 2-imidazoyl, 4-imidazolyl, pyrazinyl, 2-oxazolyl, 4-oxazolyl, 2-phenyl-4-oxazolyl, 5-oxazoyl, 3-isoxazoyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxyl)methyl, 2-pyridyloxy)methyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, ═NR′, ═N—OR′, —NR′R″, —SR′, -halogen, —SiR′R″′R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′RR′)═NR″, —NR—C(NR′R)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R′, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When Rand R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂CH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′RR′″, —OC(O)R′, —C(O)R′, —CO₂R′, —CONR′R″, —OC(O)NR′R—, —NR—C(O)R′, —NR′—C(O)NRR′″, —NR″C(O)₂R′, —NR—C(NR′R)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣB—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₈) akyl.

As used herein, the term “heteroatom” includes oxygen (O), nitrogen (N), sulfur (S), phosphorus (P) and silicon (Si).

### II. The Compositions

In one aspect, the present invention provides methods and compositions for determining or predicting patients that are most likely to respond (e.g., with a therapeutic benefit) to therapy using an Wnt inhibitor or a drug having substantially similar biological activity as the Wnt inhibitor, as well as to determine or predict patients that are most likely not to respond to therapy using an Wnt inhibitor.

In some embodiments, the Wnt inhibitor is a Porcupine inhibitor suitable for use in humans. The Wnt inhibitor may be a Porcupine inhibitor that has a function similar to a known Porcupine inhibitor such as IWP-2, IWP-3 or IWP-4, which are described by Chen B at al. (2009) Nature Chem. Biol. 5:100-107 and commercially available from Miltenyi Biotech as Stemolecule™ Wnt Inhibitor IWP-2 (#130-095-584), Stemolecule™ Wnt Inhibitor IWP-3 (#130-095-585) and Stemolecule™ Wnt Inhibitor IWP-4. Stemolecule™ IWP-2, Stemolecule™ IWP-3, and Stemolecule™ IWP-4 prevent palmitoylation of Wnt proteins by Porcupine (PORCN), a membrane-bound O— acyltransferase.

Alternatively, Wnt inhibitors can be the products of drug design and can be produced using various methods known in the art. See, international patent application WO2010/101849, published 10 Sep. 2010. Various methods of drug design, useful to design mimetics or other compounds useful in the invention are disclosed in Maulik of al. (1997) Molecular Biotechnology: Therapeutic Applications and Strategies. Wiley-Liss, Inc. (incorporated by reference in its entirety). A Wnt inhibitor can be obtained from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See, for example, Maulik et al. (1997) Molecular Biotechnology: Therapeutic Applications and Strategies. Wiley-Liss, Inc. In a molecular diversity strategy, large compound libraries are synthesized, for example, from peptides, oligonucleotides, natural or synthetic steroidal compounds, carbohydrates or natural or synthetic organic and non-steroidal molecules, using biological, enzymatic or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik et al. (1997) Molecular Biotechnology: Therapeutic Applications and Strategies. Wiley-Liss, Inc.

In another aspect, the present invention provides a compound as Porcupine antagonist or inhibitor.

By “PORCN” herein is meant Porcupine, a membrane-bound acyltransferase, required for Wnt post-translational modification. Unless specifically stated otherwise, PORCN as used herein, refers to human PORCN-accession numbers NM_017617.3/NP_060087.

In some embodiments, the Porcupine inhibitor has the structure of Formula (1):

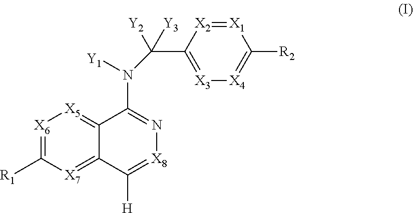

or a physiologically acceptable sat thereof, wherein,
X₁, X₂, X₃, X₄, X₅, X₆, X₇, X₈ are independently CR4 or N
Y₁ is hydrogen or CR₄;
Y₂, Y₃ are independently hydrogen, halo or CR₃;
R₁ is morpholinyl, piperazinyl, quinolinyl,

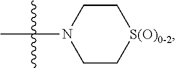

aryl, C₁₋₆ heterocycle, 5 or 6 membered heteroaryl containing 1-2 heteroatoms selected from N, O and S;
R₂ is hydrogen, halo, morpholinyl, piperazinyl, quinolinyl,

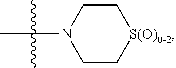

aryl, C₁₋₆ heterocycle, 5 or 6 membered heteroaryl containing 1-2 heteroatoms selected from N, O and S;
wherein 5 or 6 membered heteroaryl includes the following selected groups but is not limited to:

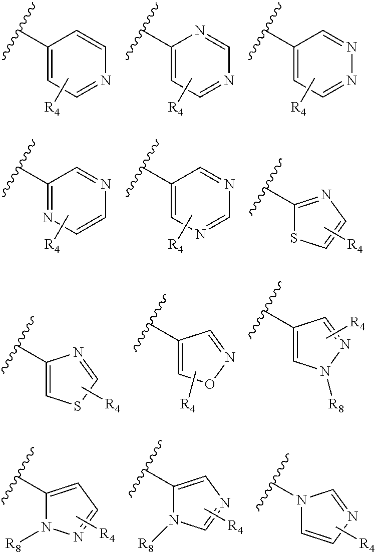

R₁ and R₂ could be independently and optionally substituted with 1-2 R₄ groups;
R₃ is hydrogen, halo, cyano, C₁₋₆ alkyl, C₁₋₆ alkoxy optionally substituted with halo, amino, hydroxyl, alkoxy or cyano;
R₄ is hydrogen, halo, C₁₋₆ alkoxy, —S(O)₂R₅, —C(O)OR₅, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano;
R₈ is hydrogen or C₁₋₆ alkyl.

As used herein, an H atom in any substituent groups (e.g., CH₂) encompasses all suitable isotopic variations, e.g., H, ²H and ³H.

As used herein, other atoms in any substituent groups encompasses all suitable isotopic variations, including but not limited to 11C, ¹³C, ¹⁴C, ¹⁵N, ¹⁷O, ¹⁸O, ³⁵S, ¹⁸F, ³⁶I and/or ¹²³I.

In some embodiments, example of the compound of the invention includes but is not limited to:
6-(2-methylpyridin-4-yl)-N-(4-(2-methylpyridin-4-yl)benzyl)-2,7-naphthyridin-1-amine;
N-(3-methyl-4-(2-methylpyridin-4-yl)benzyl)-6-(2-methylpyridin-4-yl)-2,7-naphthyridin-1-amine;
6-(3-fluorophenyl)-N-((2′-methyl-2,4′-bipyridin-5-yl)methyl)isoquinolin-1-amine;
2-(2-methylpyridin-4-yl)-N-(4-(2-methylpyridin-4-yl)benzyl)-1,6-naphthyridin-5-amine;
N-(4-(2-methylpyridin-4-yl)benzyl)-2-phenylpyrido[4,3-b]pyrazin-5-amine;
N-(4-(2-methylpyridin-4-yl)benzyl)-6-(pyridin-4-yl)-2,7-naphthyridin-1-amine;
N-(4-(2-methylpyridin-4-yl)benzyl)-6-phenyl-2,7-naphthyridin-1-amine;
6-(3-chlorophenyl)-N-(4-(2-methylpyridin-4-yl)benzyl)-2,7-naphthyridin-1-amine;
6-(3-fluorophenyl)-N-(4-(2-methylpyridin-4-yl)benzyl)-2,7-naphthyridin-1-amine;
6-(3-fluorophenyl)-N-((2′-methyl-2,4′-bipyridin-5-yl)methyl)-2,7-naphthyridin-1-amine;
6-(3-fluorophenyl)-N-(4-(2-(trifluoromethyl)pyridin-4-yl)benzyl)-2,7-naphthyridin-1-amine;
N-((2′,3-dimethyl-2,4′-bipyridin-5-yl)methyl)-6-(3-fluorophenyl)-2,7-naphthyridin-1-amine;
N-(4-(2-methylpyridin-4-yl)benzyl)-6-(pyrimidin-5-yl)-2,7-naphthyridin-1-amine;
6-(5-methylpyridin-3-yl)-N-(4-(2-methylpyridin-4-yl)benzyl)-2,7-naphthyridin-1-amine;
6-(6-methylpyridin-3-yl)-N-(4-(2-methylpyridin-4-yl)benzyl)-2,7-naphthyridin-1-amine;
3-(8-(4-(2-methylpyridin-4-yl)benzylamino)-2,7-naphthyridin-3-yl)benzonitrile;
4-(8-(4-(2-methylpyridin-4-yl)benzylamino)-2,7-naphthyridin-3-yl)benzonitrile;
6-(4-fluorophenyl)-N-(4-(2-methylpyridin-4-yl)benzyl)-2,7-naphthyridin-1-amine;
N-(4-(2-methylpyridin-4-yl)benzyl)-6-m-tolyl-2,7-naphthyridin-1-amine;
N-(4-(2-methylpyridin-4-yl)benzyl)-2,7-naphthyridin-1-amine;
N-(4-(2-methylpyridin-4-yl)benzyl)-6-(pyridin-2-yl)-2,7-naphthyridin-1-amine;
6-(2-fluoropyridin-4-yl)-N-(4-(2-methylpyridin-4-yl)benzyl)-2,7-naphthyridin-1-amine;
6-(2-fluorophenyl)-N-(4-(2-methylpyridin-4-yl)benzyl)-2,7-naphthyridin-1-amine;
N-(4-(2-methylpyridin-4-yl)benzyl)-6-(pyridin-3-yl)-2,7-naphthyridin-1-amine;
N-(biphenyl-4-ylmethyl)-6-(2-methylpyridin-4-yl)-2,7-naphthyridin-1-amine;
6-(2-methylpyridin-4-yl)-N-((5-phenylpyridin-2-yl)methyl)-2,7-naphthyridin-1-amine;
6-(3-fluorophenyl)-N-((2′-(trifluoromethyl)-2,4′-bipyridin-5-yl)methyl)-2,7-naphthyridin-1-amine;
N-(3-fluoro-4-(2-fluoropyridin-4-yl)benzyl)-6-(2-methylpyridin-4-yl)-2,7-naphthyridin-1-amine;
6-(2-methylpyridin-4-yl)-N-((2′-(trifluoromethyl)-2,4′-bipyridin-5-yl)methyl)-2,7-naphthyridin-1-amine;
N-((3-fluoro-2′-(trifluoromethyl)-2,4′-bipyridin-5-yl)methyl)-6-(2-methylpyridin-4-yl)-2,7-naphthyridin-1-amine;
N-(3-fluoro-4-(2-methylpyridin-4-yl))benzyl)-6-(2-methylpyridin-4-yl)-2,7-naphthyridin-1-amine;
N-((2′-fluoro-2,4′-bipyridin-5-yl)methyl)-6-(2-methylpyridin-4-yl)-2,7-naphthyridin-1-amine;
4-(5-(((6-(2-methylpyridin-4-yl)-2,7-naphthyridin-1-yl)amino)methyl)pyridine-2-yl)thiomorpholine 1,1-dioxide;
6-(2-methylpyridin-4-yl)-N-(4-(pyridazin-4-yl)benzyl)-2,7-naphthyridin-1-amine;
N-(4-(2-methylpyridin-4-yl)benzyl)-6-(pyrazin-2-yl)-2,7-naphthyridin-1-amine;
N-(4-(2-methylpyridin-4-yl)benzyl)-6-(pyridazin-4-yl)-2,7-naphthyridin-1-amine;
N-(4-(2-methylpyridin-4-yl)benzyl)-8-morpholino-2,7-naphthyridin-1-amine;
6-(4-methylpiperazin-1-yl)-N-(4-(2-methylpyridin-4-yl)benzyl)-2,7-naphthyridin-1-amine;
4-(8-((4-(2-methylpyridin-4-yl)benzyl)amino)-2,7-naphthyridin-3-yl)thiomorpholine 1,1-dioxide;
N-(3-fluoro-4-(2-fluoropyridin-4-yl)benzyl)-6-(3-fluorophenyl)-2,7-naphthyridin-1-amine;
N-(3-fluoro-4-(2-methylpyridin-4-yl)benzyl)-6-(3-fluorophenyl)-2,7-naphthyridin-1-amine;
N-((3-fluoro-2′-(trifluoromethyl)-2,4′-bipyridin-5-yl)methyl)-6-(3-fluorophenyl)-2,7-naphthyridin-1-amine;
N-((2′-fluoro-2,4′-bipyridin-5-yl)methyl)-6-(3-fluorophenyl)-2,7-naphthyridin-1-amine;
6-(3-fluorophenyl)-N-(3-methyl-4-(2-methylpyridin-4-yl)benzyl)-2,7-naphthyridin-1-amine;
4-(5-(((6-(3-fluorophenyl)-2,7-naphthyridin-1-yl)amino)methyl)pyridine-2-yl)thiomorpholine 1,1-dioxide;
N-(4-chlorobenzyl)-6-(2-methylpyridin-4-yl)-2,7-naphthyridin-1-amine;
N-(4-methylbenzyl)-6-(2-methylpyridin-4-yl)-2,7-naphthyridin-1-amine;
6-(2-methylpyridin-4-yl)-N-(pyridin-3-ylmethyl)-2,7-naphthyridin-1-amine;
N-benzyl-2-(3-fluorophenyl)-1,6-naphthyridin-5-amine;
2-(3-fluorophenyl)-N-((2′-methyl-2,4′-bipyridin-5-yl)methyl)-1,6-naphthyridin-5-amine;
N-((2′-methyl-2,4′-bipyridin-5-yl)methyl)-2-(2-methylpyridin-4-yl)-1,6-naphthyridin-5-amine;
N-((6-(3-fluorophenyl)pyridin-3-yl)methyl)-2-(2-methylpyridin-4-yl)-1,6-naphthyridin-5-amine;
N-(4-(2-fluoropyridin-4-yl)benzyl)-2-(2-methylpyridin-4-yl)-1,6-naphthyridin-5-amine;
2-(2-methylpyridin-4-yl)-N-(4-(2-(trifluoromethyl)pyridin-4-yl)benzyl)-1,6-naphthyridin-5-amine;
N-((2′,3-dimethyl-2,4′-bipyridin-5-yl)methyl)-2-(2-methylpyridin-4-yl)-1,6-naphthyridin-5-amine;
N-(biphenyl-4-ylmethyl)-6-(3-fluorophenyl)isoquinolin-1-amine;
N-((2-fluorobiphenyl-4-yl)methyl)-6-(3-fluorophenyl)isoquinolin-1-amine;
N-((2′-methyl-2,4′-bipyridin-5-yl)methyl)-6-phenylisoquinolin-1-amine;
6-(3-chlorophenyl)-N-((2′-methyl-2,4′-bipyridin-5-yl)methyl)isoquinolin-1-amine;
N-(4-(2-methylpyridin-4-yl)benzyl)-6-phenylisoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-4-yl)isoquinolin-1-amine;
6-(6-methylpyridin-3-yl)-N-(4-(2-methylpyridin-4-yl)benzyl)isoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-3-yl)isoquinolin-1-amine;
N-(4-(2-methylpyridin-4-yl)benzyl)-6-(pyrazin-2-yl)isoquinolin-1-amine;
N-(4-(2-methylpyridin-4-yl)benzyl)-6-(pyridazin-4-yl)isoquinolin-1-amine;
N-((2′-methyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyridin-2-yl)isoquinolin-1-amine;
N-((2′,3-dimethyl-2,4′-bipyridin-5-yl)methyl)-6-(3-fluorophenyl)isoquinolin-1-amine;
N-((2′,3-dimethyl-2,4′-bipyridin-5-yl)methy-6-(5-methylpyridin-3-yl)isoquinolin-1-amine;
N-((2′-methyl-2,4′-bipyridin-5-yl)methyl)-2-phenylpyrido[4,3-b]pyrazin-5-amine;
2-(3-fluorophenyl)-N-(4-(2-methylpyridin-4-yl)benzyl)pyrido[4,3-b]pyrazin-5-amine;
2-(3-fluorophenyl)-N-((2′-methyl-2,4′-bipyridin-5-yl)methyl)pyrido[4,3-b]pyrazin-5-amine;
2-(3-fluorophenyl)-N-(3-methyl-4-(2-methylpyridin-4-yl)benzyl)pyrido[4,3-b]pyrazin-5-amine;
N-(3-fluoro-4-(2-methylpyridin-4-yl)benzyl)-2-(3-fluorophenyl)pyrido[4,3-b]pyrazin-5-amine;
2-(2-methylpyridin-4-yl)-N-(4-(2-methylpyridin-4-yl)benzyl)pyrido[4,3-b]pyrazin-5-amine;
N-((2′-methyl-2,4′-bipyridin-5-yl)methyl)-2-(2-methylpyridin-4-yl)pyrido[4,3-b]pyrazin-5-amine;
N-(3-methyl-4-(2-methylpyridin-4-yl)benzyl)-2-(2-methylpyridin-4-yl)pyrido[4,3-b]pyrazin-5-amine;
N-(3-fluoro-4-(2-methylpyridin-4-yl)benzyl)-2-(2-methylpyridin-4-yl)pyrido[4,3-b]pyrazin-5-amine;
N-((2′,3-dimethyl-2,4′-bipyridin-5-yl)methyl)-6-(pyrazin-2-yl)-2,7-naphthyridin-1-amine;
6-(2-methylmorpholino)-N-(4-(2-methylpyridin-4-yl)benzyl)-2,7-naphthyridin-1-amine;
(S)-6-(2-methylmorpholino)-N-(4-(2-methylpyridin-4-yl)benzyl)-2,7-naphthyridin-1-amine;
(R)-6-(2-methylmorpholino)-N-(4-(2-methylpyridin-4-yl)benzyl)-2,7-naphthyridin-1-amine;
1-(4-(8-(4-(2-methylpyridin-4-yl)benzylamino)-2,7-naphthyridin-3-yl) piperazin-1-yl)ethanone;
6-(1H-imidazol-1-yl)-N-(4-(2-methylpyridin-4-yl)benzyl)-2,7-naphthyridin-1-amine;
6-(4-methyl-1H-imidazol-1-yl)-N-(4-(2-methylpyridin-4-yl)benzyl)-2,7-naphthyridin-1-amine;
N-(4-(2-methylpyridin-4-yl)benzyl)-6-(1H-tetrazol-5-yl)-2,7-naphthyridin-1-amine;
6-(5-methyl-1,3,4-oxadiazol-2-yl)-N-(4-(2-methylpyridin-4-yl)benzyl)-2,7-naphthyridin-1-amine;
6-(1-methyl-1H-pyrazol-3-yl)-N-(4-(2-methylpyridin-4-yl)benzyl)-2,7-naphthyridin-1-amine;
N-(4-(2-methylpyridin-4-yl)benzyl)-6-(thiazol-5-yl)-2,7-naphthyridin-1-amine;
N-(4-(2-methylpyridin-4-yl)benzyl)-6-(oxazol-5-yl)-2,7-naphthyridin-1-amine;
N-((2′,3-dimethyl-2,4′-bipyridin-5-yl)methyl)-6-(5-methylpyridin-3-yl)-2,7-naphthyridin-1-amine;
N-((2′,3-dimethyl-2,4′-bipyridin-5-yl)methyl)-6-(2-methylpyridin-4-yl)-2,7-naphthyridin-1-amine;
N-((3-fluoro-2′-methyl-2,4′-bipyridin-5-yl)methyl)-6-(2-methylpyridin-4-yl)-2,7-naphthyridin-1-amine;
N-((2′,3-dimethyl-2,4′-bipyridin-5-yl)methyl)-6-(5-fluoropyridin-3-yl)-2,7-naphthyridin-1-amine;
N-(3-methyl-4-(2-methylpyridin-4-yl)benzyl)-6-(pyrazin-2-yl)-2,7-naphthyridin-1-amine;
N-(3-fluoro-4-(2-methylpyridin-4-yl)benzyl)-6-(pyrazin-2-yl)-2,7-naphthyridin-1-amine; methyl 4-(8-(4-(2-methylpyridin-4-yl)benzylamino)-2,7-naphthyridin-3-yl)piperazine-1-carboxylate;
4-(8-(4-(2-methylpyridin-4-yl)benzylamino)-2,7-naphthyridin-3-yl)piperazin-2-one;
2-(4-(8-(4-(2-methylpyridin-4-yl)benzylamino)-2,7-naphthyridin-3-yl)piperazin-1-yl)acetonitrile;
2-methyl-4-(4-((8-(2-methylpyridin-4-yl)-2,7-naphthyridin-1-ylamino)methyl)phenyl)pyridine 1-oxide;
6-(2-chloropyridin-4-yl)-N-((2′,3-dimethyl-2,4′-bipyridin-5-yl)methyl)-2,7-naphthyridin-1-amine;
6-(2-chloropyridin-4-yl)-N-(4-(2-methylpyridin-4-yl)benzyl)-2,7-naphthyridin-1-amine;
2-(2-methylpyridin-4-yl)-5-((8-(2-methylpyridin-4-yl)-2,7-naphthyridin-1-ylamino)methyl)benzonitrile;
N-(3-methoxy-4-(2-methylpyridin-4-yl)benzyl)-6-(2-methylpyridin-4-yl)-2,7-naphthyridin-1-amine;
N-((3-chloro-2′-methyl-2,4′-bipyridin-5-yl)methyl)-6-(2-methylpyridin-4-yl)-2,7-naphthyridin-1-amine;
2′-methyl-5-((6-(2-methylpyridin-4-yl)-2,7-naphthyridin-1-ylamino)methyl)-2,4′-bipyridine-3-carbonitrile; and N-(4-(2-(difluoromethyl)pyridin-4-yl)benzyl)-6-(2-methylpyridin-4-yl)-2,7-naphthyridin-1-amine; or physiologically acceptable salts thereof.

In some embodiments, examples of the compound of the invention include but are not limited to the compounds provided in Examples 1-5 and Table 1. A person skilled in the art can clearly understand and know that the other compounds could be prepared by the same strategy as Examples 1-5.

TABLE 1
Compounds Table

| No. | Compound Structure | Compound physical characterization |
|---|---|---|
| 6 | 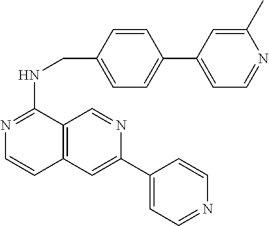 [SMILES: Cc1cc(-c2ccc(CNc3nccc4cc(-c5ccncc5)ncc34)cc2)ccn1] | MS m/z = 404.2 (M + 1); |
| 7 | 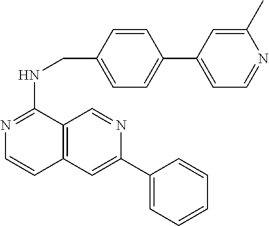 [SMILES: Cc1cc(-c2ccc(CNc3nccc4cc(-c5ccccc5)ncc34)cc2)ccn1] | MS m/z = 403.2 (M + 1); |
| 8 | 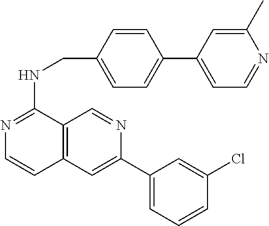 [SMILES: Cc1cc(-c2ccc(CNc3nccc4cc(-c5cccc(Cl)c5)ncc34)cc2)ccn1] | MS m/z = 437.2 (M + 1); |
| 9 | 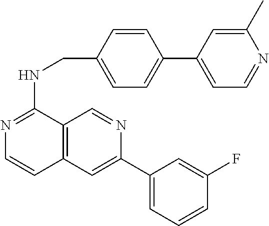 [SMILES: Cc1cc(-c2ccc(CNc3nccc4cc(-c5cccc(F)c5)ncc34)cc2)ccn1] | MS m/z = 421.2 (M + 1); ¹H NMR (400 MHz, DMSO-d6) δ 9.82 (s, 1H), 8.76 (d, J = 6.0 Hz, 1H), 8.39 (s, 1H), 8.17 (s, 1H), 7.95-8.18 (m, 6H), 7.58-7.66 (m, 3H), 7.35 (t, J = 8.0 Hz, 1H), 7.07 (d, J = 6.0 Hz, 1H), 5.77 (s, 1H), 4.92 (d, J = 6.0 Hz, 1H), 2.70 (s, 3H) |
| 10 | 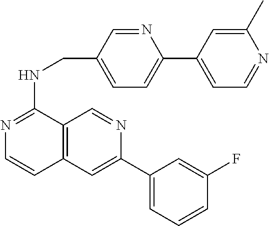 [SMILES: Cc1cc(-c2ccc(CNc3nccc4cc(-c5cccc(F)c5)ncc34)cn2)ccn1] | MS m/z = 422.2 (M + 1); |
| 11 | 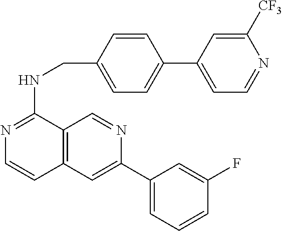 [SMILES: Fc1cccc(-c2cc3ccnc(NCc4ccc(-c5ccnc(C(F)(F)F)c5)cc4)c3cn2)c1] | MS m/z = 475.2 (M + 1); |
| 12 | 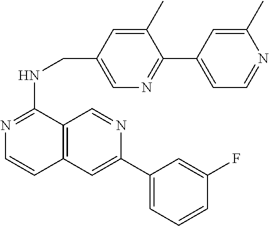 [SMILES: Cc1cc(-c2ncc(CNc3nccc4cc(-c5cccc(F)c5)ncc34)cc2C)ccn1] | MS m/z = 436.2 (M + 1); |
| 13 | 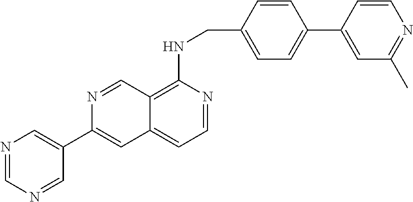 [SMILES: Cc1cc(-c2ccc(CNc3nccc4cc(-c5cncnc5)ncc34)cc2)ccn1] | MS m/z = 405.2 (M + 1); |
| 14 | 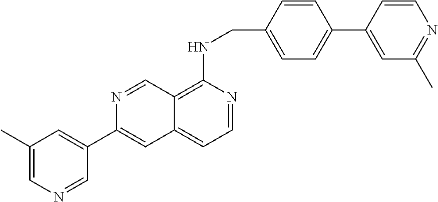 | MS m/z = 418.2 (M + 1); ¹H NMR (300 MHz, CDCl₃): δ 2.46 (s, 3H), 2.63 (s, 3H), 4.94 (d, J = 5.10 Hz, 2H), 5.94 (br, 1H), 6.97 (d, J = 5.70 Hz, 1H), 7.31 (d, J = 4.20 Hz, 1H), 7.36 (s, 1H), 7.54 (d, J = 8.10 Hz, 2H), 7.63 (d, J = 8.40 Hz, 2H), 7.90 (s, 1H), 8.19 (d, J = 6.00 Hz, 1H), 8.22 (s, 1H), 8.51 (m, 2H), 9.08 (s, 1H), 9.30 (s, 1H). |
| 15 | 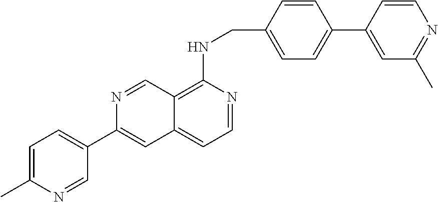 | MS m/z = 418.2 (M + 1); |
| 16 | 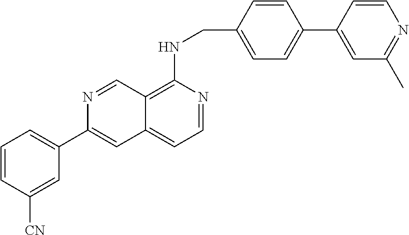 [SMILES: Cc1cc(-c2ccc(CNc3nccc4cc(-c5cccc(C#N)c5)ncc34)cc2)ccn1] | MS m/z = 428.2 (M + 1); ¹H NMR (300 MHz, CDCl₃): δ 2.64 (s, 3H), 4.96 (d, J = 5.10 Hz, 2H), 5.99 (br, 1H), 7.31 (d, J = 5.10 Hz, 1H), 7.37 (s, 1H), 7.63 (m, 1H), 7.73 (m, 1H), 7.91 (s, 1H), 8.22 (d, J = 5.70 Hz, 1H), 8.33 (m, 1H), 8.44 (s, 1H), 8.53 (d, J = 5.10 Hz, 1H), 9.33 (s, 1H). |
| 17 | 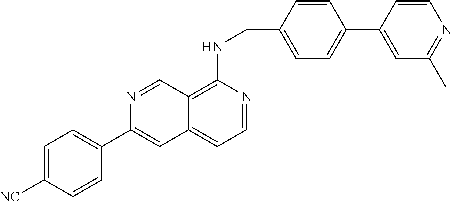 [SMILES: Cc1cc(-c2ccc(CNc3nccc4cc(-c5ccc(C#N)cc5)ncc34)cc2)ccn1] | MS m/z = 428.2 (M + 1); |
| 18 | 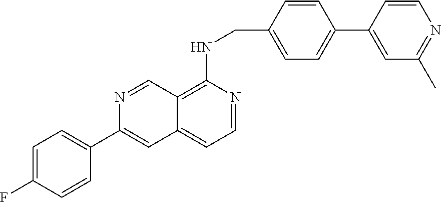 [SMILES: Cc1cc(-c2ccc(CNc3nccc4cc(-c5ccc(F)cc5)ncc34)cc2)ccn1] | MS m/z = 420.2 (M + 1); |
| 19 | 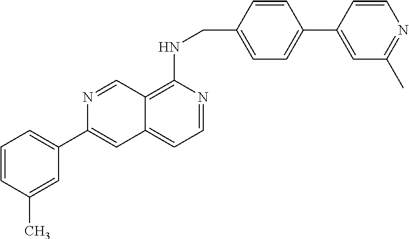 [SMILES: Cc1cccc(-c2cc3ccnc(NCc4ccc(-c5ccnc(C)c5)cc4)c3cn2)c1] | MS m/z = 417.2 (M + 1); |
| 20 | 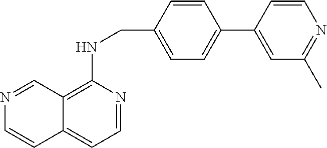 [SMILES: Cc1cc(-c2ccc(CNc3nccc4ccncc34)cc2)ccn1] | MS m/z = 326.1 (M + 1); ¹H NMR (300 MHz, CDCl₃): δ 2.58 (s, 3H), 4.90 (d, J = 5.1 Hz, 2H), 5.96 (br, 1H), 6.91 (d, J = 6.0 Hz, 1H), 7.48-7.58 (m, 4H), 7.62 (d, J = 5.7 Hz, 1H), 7.70 (d, J = 8.4 Hz, 2H), 8.02 (d, J = 5.7 Hz, 1H), 8.40 (d, J = 5.1 Hz, 1H), 8.53 (d, J = 5.7 Hz, 1H), 9.50 (s, 1H). |
| 21 | 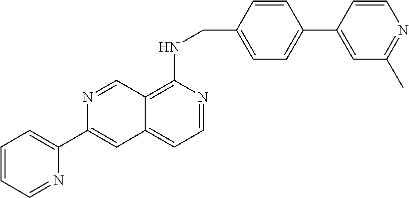 [SMILES: Cc1cc(-c2ccc(CNc3nccc4cc(-c5ccccn5)ncc34)cc2)ccn1] | MS m/z = 404.2 (M + 1); |
| 22 | 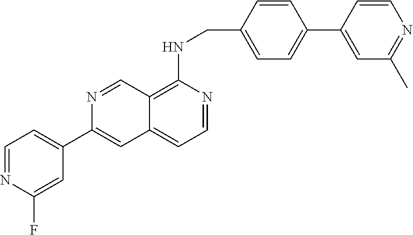 [SMILES: Cc1cc(-c2ccc(CNc3nccc4cc(-c5ccnc(F)c5)ncc34)cc2)ccn1] | MS m/z = 422.2 (M + 1); ¹H NMR (300 MHz, CDCl₃): δ 2.64 (s, 3H), 4.96 (d, J = 5.40 Hz, 2H), 5.96 (br, 1H), 7.01 (d, J = 6.00 Hz, 1H), 7.31 (m, 1H), 7.37 (s, 1H), 7.56 (d, J = 8.10 Hz, 2H), 7.64 (d, J = 8.10 Hz, 2H), 7.88 (m, 1H), 7.99 (s, 1H), 8.25 (d, J = 6.00 Hz, 1H), 8.36 (d, J = 8.10 Hz, 1H), 9.32 (s, 1H). |
| 23 | 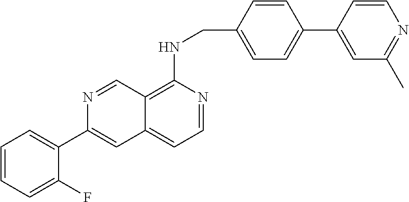 [SMILES: Cc1cc(-c2ccc(CNc3nccc4cc(-c5ccccc5F)ncc34)cc2)ccn1] | MS m/z = 421.2 (M + 1); |
| 24 | 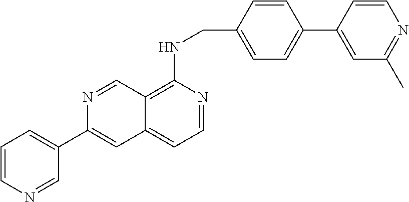 [SMILES: Cc1cc(-c2ccc(CNc3nccc4cc(-c5cccnc5)ncc34)cc2)ccn1] | MS m/z = 404.2 (M + 1); |
| 25 | 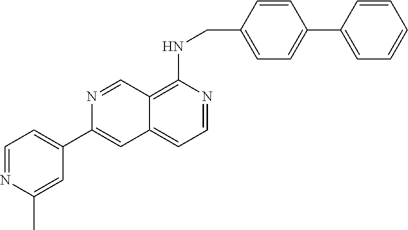 [SMILES: Cc1cc(-c2cc3ccnc(NCc4ccc(-c5ccccc5)cc4)c3cn2)ccn1] | MS m/z = 403.2 (M + 1); |
| 26 | 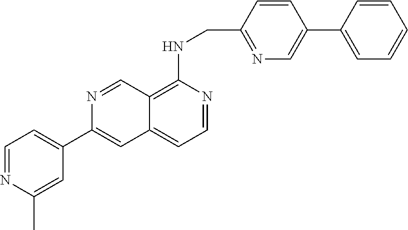 [SMILES: Cc1cc(-c2cc3ccnc(NCc4ccc(-c5ccccc5)cn4)c3cn2)ccn1] | MS m/z = 404.2 (M + 1); |
| 27 | 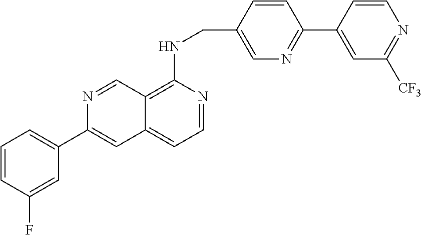 [SMILES: Fc1cccc(-c2cc3ccnc(NCc4ccc(-c5ccnc(C(F)(F)F)c5)nc4)c3cn2)c1] | MS m/z = 476.2 (M + 1); |
| 28 | 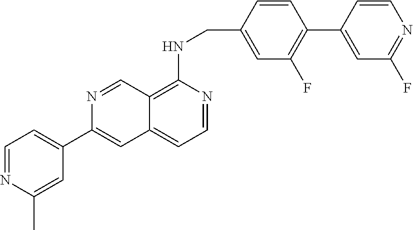 [SMILES: Cc1cc(-c2cc3ccnc(NCc4ccc(-c5ccnc(F)c5)c(F)c4)c3cn2)ccn1] | MS m/z = 440.2 (M + 1); 1H NMR (300 MHz, CDCl3): δ 2.61 (s, 3H), 4.88 (d, J = 5.70 Hz, 2H), 5.98 (br, 1H), 6.92 (d, J = 5.7 Hz, 1H), 7.02 (s, 1H), 7.26 (m, 3H), 7.37 (t, J = 7.8 Hz, 1H), 7.68 (d, J = 5.4 Hz, 1H), 7.79 (s, 1H), 7.89 (s, 1H), 8.11 (d, J = 6.0 Hz, 1H), 8.17 (d, J = 5.1 Hz, 1H), 8.55 (d, J = 5.4 Hz, 1H), 9.26 (s, 1H). |
| 29 | 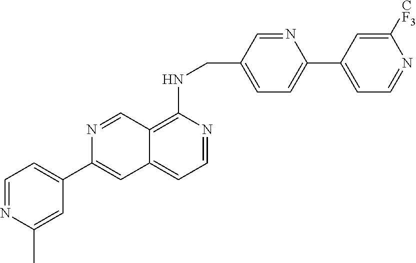 [SMILES: Cc1cc(-c2cc3ccnc(NCc4ccc(-c5ccnc(C(F)(F)F)c5)nc4)c3cn2)ccn1] | MS m/z = 473.2 (M + 1); |
| 30 | 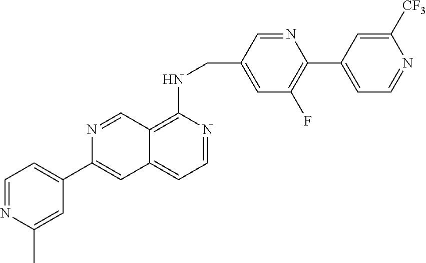 [SMILES: Cc1cc(-c2cc3ccnc(NCc4cnc(-c5ccnc(C(F)(F)F)c5)c(F)c4)c3cn2)ccn1] | MS m/z = 497.2 (M + 1); |
| 31 | 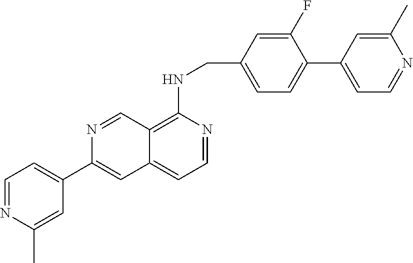 [SMILES: Cc1cc(-c2cc3ccnc(NCc4ccc(-c5ccnc(C)c5)c(F)c4)c3cn2)ccn1] | MS m/z = 436.2 (M + 1); ¹H NMR (300 MHz, CDCl₃): δ 2.63 (s, 3H), 2.70 (s, 3H), 4.96 (d, J = 5.70 Hz, 2H), 6.02 (br, 1H), 7.02 (d, J = 5.70 Hz, 1H), 7.34 (s, 1H), 7.45 (d, J = 7.80 Hz, 2H), 7.61 (s, 1H), 7.78 (d, J = 4.80 Hz, 2H), 7.88 (s, 1H), 7.98 (s, 1H), 8.22 (d, J = 5.70 Hz, 1H), 8.55 (d, J = 5.10 Hz, 2H), 8.64 (d, J = 5.10 Hz, 2H), 9.34 (s, 1H). |
| 32 | 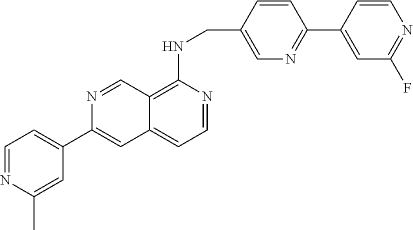 [SMILES: Cc1cc(-c2cc3ccnc(NCc4ccc(-c5ccnc(F)c5)nc4)c3cn2)ccn1] | MS m/z = 423.2 (M + 1); |
| 33 | 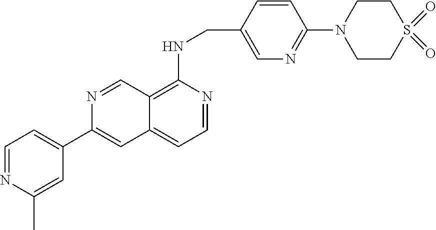 [SMILES: Cc1cc(-c2cc3ccnc(NCc4ccc(N5CCS(=O)(=O)CC5)nc4)c3cn2)ccn1] | MS m/z = 461.2 (M + 1); ¹H NMR (300 MHz, CDCl₃): δ 2.69 (s, 3H), 3.06 (t, 4H), 4.18 (t, 4H), 4.79 (d, J = 5.40 Hz, 2H), 5.85 (br, 1H), 6.76 (d, J = 8.70 Hz, 1H), 6.99 d, J = 6.00 Hz, 1H), 7.69 (q, 1H), 7.76 (q, 1H), 7.86 (s, 1H), 7.96 (s, 1H), 8.22 (d, J = 6.00 Hz, 1H), 8.31 (s, 1H), 8.63 (d, J = 5.40 Hz, 1H), 9.27 (s, 1H). |
| 34 | 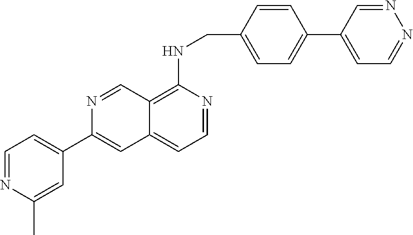 [SMILES: Cc1cc(-c2cc3ccnc(NCc4ccc(-c5ccnnc5)cc4)c3cn2)ccn1] | MS m/z = 405.2 (M + 1); |
| 35 | 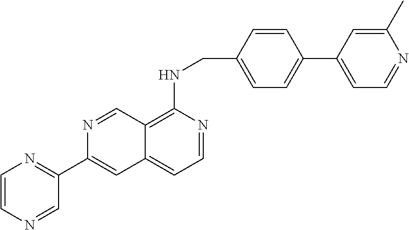 [SMILES: Cc1cc(-c2ccc(CNc3nccc4cc(-c5cnccn5)ncc34)cc2)ccn1] | MS m/z = 405.2 (M + 1); ¹H NMR (300 MHz, CDCl₃): δ 2.64 (s, 3H), 4.96 (d, J = 5.40 Hz, 2H), 5.96 (br, 1H), 7.05 (d, J = 5.70 Hz, 1H), 7.31 (m, 1H), 7.37 (s, 1H), 7.56 (d, J = 8.40 Hz, 2H), 7.64 (d, J = 8.40 Hz, 2H), 8.23 (d, J = 5.70 Hz, 1H), 8.54 (d, J = 5.40 Hz, 1H), 8.57 (s, 1H), 8.64 (d, J = 2.40 Hz, 1H), 8.67 (m, 1H), 9.32 (s, 1H), 9.71 (d, J = 1.50 Hz, 1H). |
| 36 | 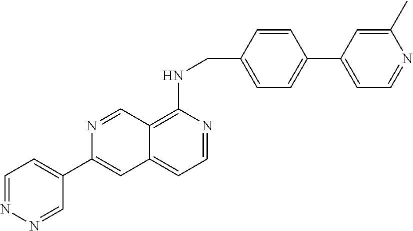 [SMILES: Cc1cc(-c2ccc(CNc3nccc4cc(-c5ccnnc5)ncc34)cc2)ccn1] | MS m/z = 405.2 (M + 1); |
| 37 | 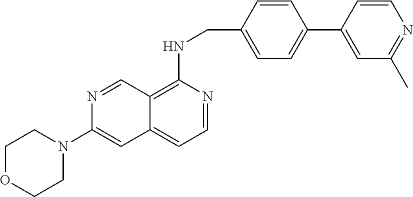 [SMILES: Cc1cc(-c2ccc(CNc3nccc4cc(N5CCOCC5)ncc34)cc2)ccn1] | MS m/z = 412.2 (M + 1); |
| 38 | 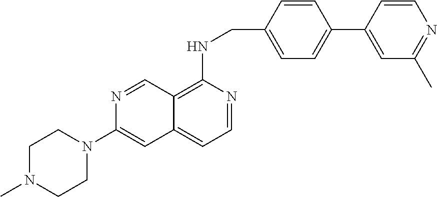 | MS m/z = 425.2 (M + 1); |
| 39 | 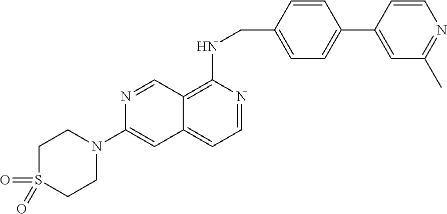 [SMILES: Cc1cc(-c2ccc(CNc3nccc4cc(N5CCS(=O)(=O)CC5)ncc34)cc2)ccn1] | MS m/z = 460.2 (M + 1); ¹H NMR (300 MHz, CD₃OD): δ 2.56 (s, 3H), 3.13 (t, 4H), 4.28 (t, 4H), 4.81 (s, 2H), 6.79 (d, J = 6.30 Hz, 1H), 6.99 (s, 1H), 7.47 (m, 2H), 7.51 (s, 1H), 7.55 (d, J = 6.60 Hz, 2H), 7.71 (d, J = 8.40 Hz, 2H), 8.38 (d, J = 5.40 Hz, 1H), 9.27 (s, 1H). |
| 40 | 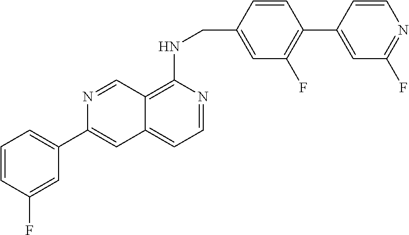 [SMILES: Fc1cccc(-c2cc3ccnc(NCc4ccc(-c5ccnc(F)c5)c(F)c4)c3cn2)c1] | MS m/z = 443.2 (M + 1); |
| 41 | 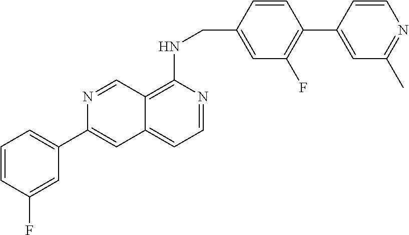 [SMILES: Cc1cc(-c2ccc(CNc3nccc4cc(-c5cccc(F)c5)ncc34)cc2F)ccn1] | MS m/z = 439.2 (M + 1); |
| 42 | 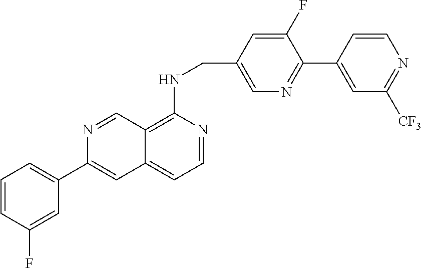 [SMILES: Fc1cccc(-c2cc3ccnc(NCc4cnc(-c5ccnc(C(F)(F)F)c5)c(F)c4)c3cn2)c1] | MS m/z = 494.2 (M + 1); |
| 43 | 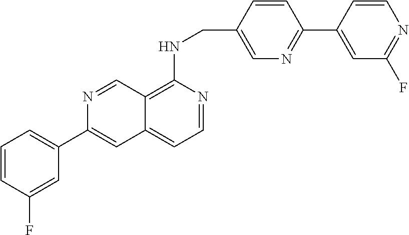 [SMILES: Fc1cccc(-c2cc3ccnc(NCc4ccc(-c5ccnc(F)c5)nc4)c3cn2)c1] | MS m/z = 426.2 (M + 1); |
| 44 | 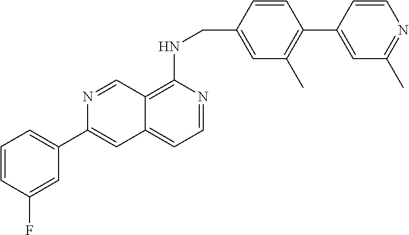 [SMILES: Cc1cc(-c2ccc(CNc3nccc4cc(-c5cccc(F)c5)ncc34)cc2C)ccn1] | MS m/z = 435.2 (M + 1); |
| 45 | 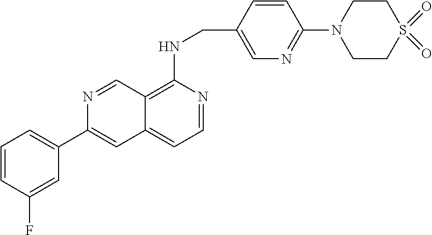 [SMILES: O=S1(=O)CCN(c2ccc(CNc3nccc4cc(-c5cccc(F)c5)ncc34)cn2)CC1] | MS m/z = 464.2 (M + 1); |
| 46 | 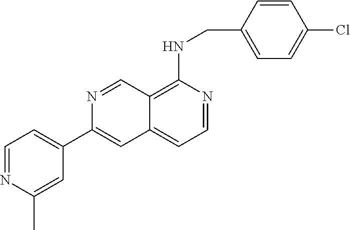 [SMILES: Cc1cc(-c2cc3ccnc(NCc4ccc(Cl)cc4)c3cn2)ccn1] | MS m/z = 361.2 (M + 1); |
| 47 | 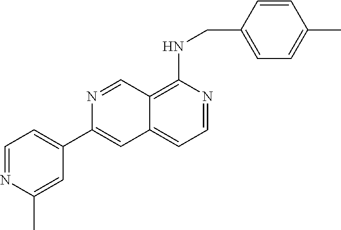 [SMILES: Cc1ccc(CNc2nccc3cc(-c4ccnc(C)c4)ncc23)cc1] | MS m/z = 341.1 (M + 1); ¹H NMR (300 MHz, CD₃OD): δ 2.31 (s, 3H), 2.65 (s, 3H), 4.76 (s, 2H), 6.98 (m, 1H), 7.12 (d, J = 7.80 Hz, 2H), 7.28 (d, J = 8.10 Hz, 2H), 7.92 (m, 1H), 8.03 (m, 2H), 8.17 (s, 1H), 8.52 (d, J = 5.40 Hz, 1H), 9.56 (s, 1H). |
| 48 | 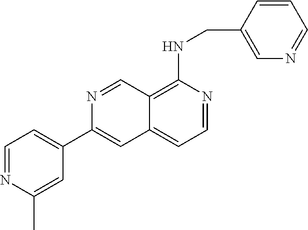 [SMILES: Cc1cc(-c2cc3ccnc(NCc4cccnc4)c3cn2)ccn1] | MS m/z = 328.1 (M + 1); |
| 49 | 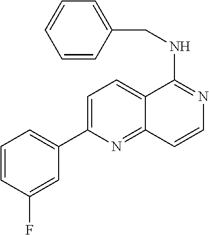 [SMILES: Fc1cccc(-c2ccc3c(NCc4ccccc4)nccc3n2)c1] | MS m/z = 330.1 (M + 1); |
| 50 | 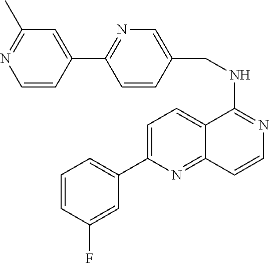 [SMILES: Cc1cc(-c2ccc(CNc3nccc4nc(-c5cccc(F)c5)ccc34)cn2)ccn1] | MS m/z = 422.2 (M + 1); ¹H NMR (400 MHz, DMSO-d6) δ 8.96 (d, J = 8.4 Hz, 1H), 8.87 (s, 1H), 8.76 (d, J = 6.0 Hz, 1H), 802-8.37 (m, 8H), 7.61-7.67 (m, 1H), 7.42 (t, J = 8.0 Hz, 1H), 7.19 (d, J = 6.4 Hz, 1H), 5.76 (s, 1H), 4.93 (d, J = 5.6 Hz, 2H), 2.69 (s, 3H). |
| 51 | 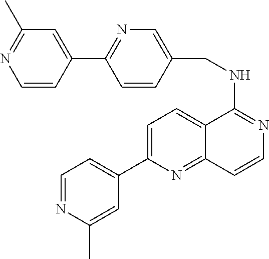 [SMILES: Cc1cc(-c2ccc(CNc3nccc4nc(-c5ccnc(C)c5)ccc34)cn2)ccn1] | MS m/z = 419.2 (M + 1); |
| 52 | 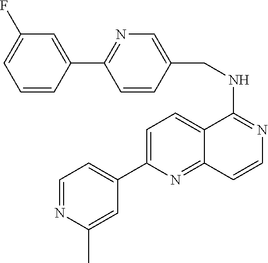 [SMILES: Cc1cc(-c2ccc3c(NCc4ccc(-c5cccc(F)c5)nc4)nccc3n2)ccn1] | MS m/z = 422.2 (M + 1); |
| 53 | 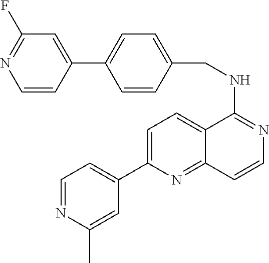 [SMILES: Cc1cc(-c2ccc3c(NCc4ccc(-c5ccnc(F)c5)cc4)nccc3n2)ccn1] | MS m/z = 422.2 (M + 1); |
| 54 | 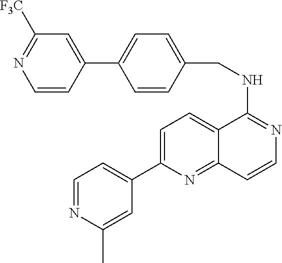 [SMILES: Cc1cc(-c2ccc3c(NCc4ccc(-c5ccnc(C(F)(F)F)c5)cc4)nccc3n2)ccn1] | MS m/z = 472.2 (M + 1); |
| 55 | 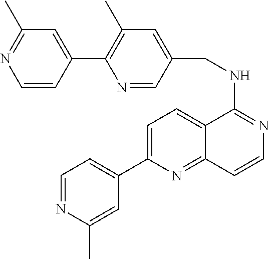 [SMILES: Cc1cc(-c2ccc3c(NCc4cnc(-c5ccnc(C)c5)c(C)c4)nccc3n2)ccn1] | MS m/z = 433.2 (M + 1); |
| 56 | 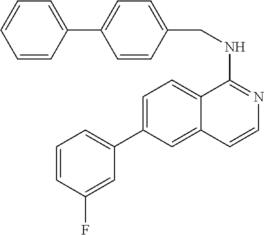 [SMILES: Fc1cccc(-c2ccc3c(NCc4ccc(-c5ccccc5)cc4)nccc3c2)c1] | MS m/z = 405.2 (M + 1); |
| 57 | 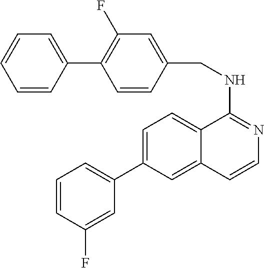 [SMILES: Fc1cccc(-c2ccc3c(NCc4ccc(-c5ccccc5)c(F)c4)nccc3c2)c1] | MS m/z = 423.2 (M + 1); |
| 58 | 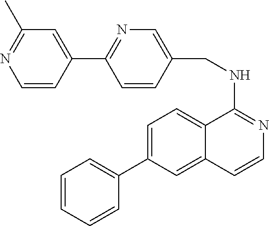 [SMILES: Cc1cc(-c2ccc(CNc3nccc4cc(-c5ccccc5)ccc34)cn2)ccn1] | MS m/z = 403.2 (M + 1); |
| 59 | 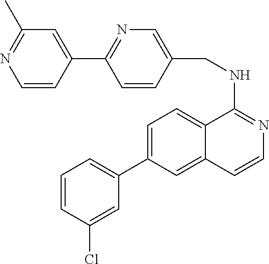 [SMILES: Cc1cc(-c2ccc(CNc3nccc4cc(-c5cccc(Cl)c5)ccc34)cn2)ccn1] | MS m/z = 437.2 (M + 1); |
| 60 | 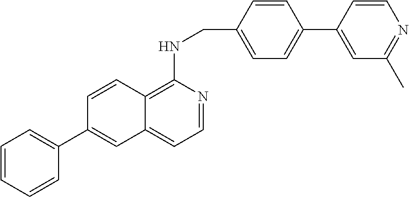 [SMILES: Cc1cc(-c2ccc(CNc3nccc4cc(-c5ccccc5)ccc34)cc2)ccn1] | MS m/z = 402.2 (M + 1); |
| 61 | 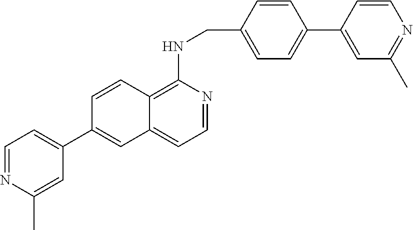 [SMILES: Cc1cc(-c2ccc(CNc3nccc4cc(-c5ccnc(C)c5)ccc34)cc2)ccn1] | MS m/z = 417.2 (M + 1); 1H NMR (300 MHz, CDCl3): δ 2.45 (s, 3H), 2.64 (s, 3H), 4.94 (d, J = 5.10 Hz, 2H), 5.93 (br, 1H), 7.00 (d, J = 5.70 Hz, 1H), 7.32 (d, J = 5.10 Hz, 1H), 7.36 (s, 1H), 7.54 (d, J = 8.10 Hz, 2H), 7.63 (d, J = 8.10 Hz, 2H), 7.80 (m, 2H), 8.20 (d, J = 6.00 Hz, 1H), 8.21 (s, 1H), 8.53 (m, 2H), 9.10 (s, 1H), 9.31 (s, 1H). |
| 62 | 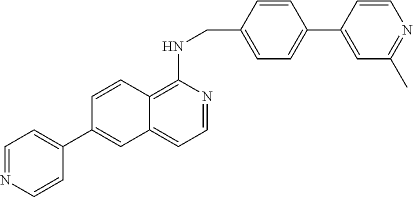 [SMILES: Cc1cc(-c2ccc(CNc3nccc4cc(-c5ccncc5)ccc34)cc2)ccn1] | MS m/z = 403.2 (M + 1); |
| 63 | 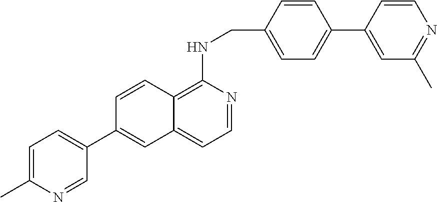 | MS m/z = 417.2 (M + 1); ¹H NMR (300 MHz, CDCl₃): δ 2.63 (s, 3H), 2.65 (s, 3H), 4.93 (d, J = 5.10 Hz, 2H), 7.06 (d, J = 6.00 Hz, 1H), 7.30 (m, 2H), 7.37 (s, 1H), 7.55 (d, J = 8.10 Hz, 2H), 7.63 (d, J = 8.10 Hz, 2H), 7.67 (m, 1H), 7.88 (m, 3H), 8.07 (d, J = 6.00 Hz, 1H), 8.53 (d, J = 5.10 Hz, 1H), 8.82 (d, J = 2.40 Hz, 1H). |
| 64 | 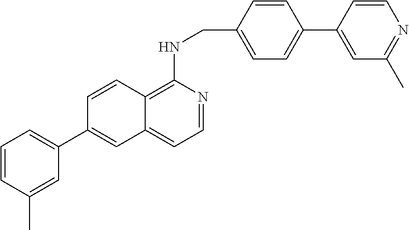 [SMILES: Cc1cccc(-c2ccc3c(NCc4ccc(-c5ccnc(C)c5)cc4)nccc3c2)c1] | MS m/z = 416.2 (M + 1); |
| 65 | 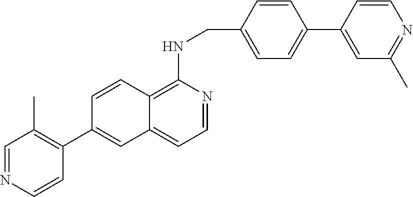 [SMILES: Cc1cc(-c2ccc(CNc3nccc4cc(-c5ccncc5C)ccc34)cc2)ccn1] | MS m/z = 417.2 (M + 1); |
| 66 | 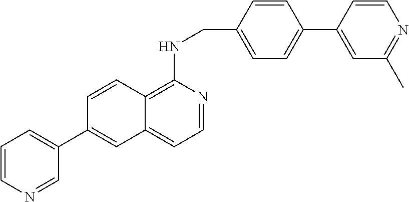 [SMILES: Cc1cc(-c2ccc(CNc3nccc4cc(-c5cccnc5)ccc34)cc2)ccn1] | MS m/z = 403.2 (M + 1); |
| 67 | 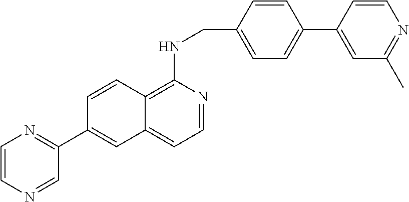 [SMILES: Cc1cc(-c2ccc(CNc3nccc4cc(-c5cnccn5)ccc34)cc2)ccn1] | MS m/z = 404.2 (M + 1); |
| 68 | 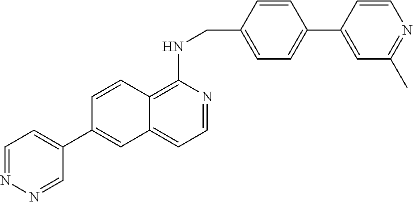 [SMILES: Cc1cc(-c2ccc(CNc3nccc4cc(-c5ccnnc5)ccc34)cc2)ccn1] | MS m/z = 404.2 (M + 1); |
| 69 | 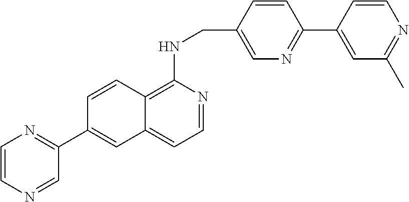 [SMILES: Cc1cc(-c2ccc(CNc3nccc4cc(-c5cnccn5)ccc34)cn2)ccn1] | MS m/z = 405.2 (M + 1); ¹H NMR (400 MHz, DMSO-d6) δ 9.52 (d, J = 1.2 Hz, 1H), 8.92 (d, J = 2.0 Hz, 1H), 8.84-8.86 (m, 1H), 8.75- 8.82 (m, 4H), 8.56 (d, J = 8.8 Hz, 1H), 8.42 (s, 1H), 8.31 (d, J = 8.8 Hz, 2H), 8.12 (d, J = 8.0 Hz, 1H), 7.78 (d, J = 6.8 Hz, 1H), 7.40 (d, J = 6.8 Hz, 1H), 5.76 (s, 1H), 5.00 (d, J = 5.6 Hz, 2H), 2.73 (s, 1H). |
| 70 | 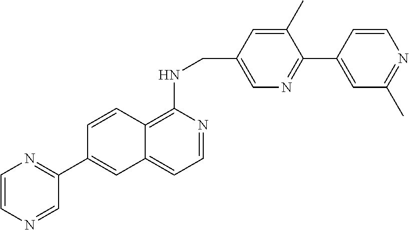 [SMILES: Cc1cc(-c2ncc(CNc3nccc4cc(-c5cnccn5)ccc34)cc2C)ccn1] | MS m/z = 419.2 (M + 1); |
| 71 | 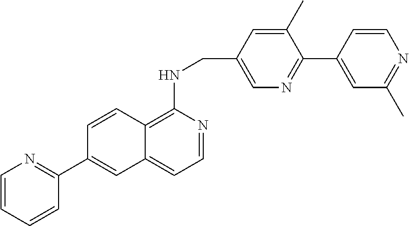 [SMILES: Cc1cc(-c2ncc(CNc3nccc4cc(-c5ccccn5)ccc34)cc2C)ccn1] | MS m/z = 418.2 (M + 1); |
| 72 | 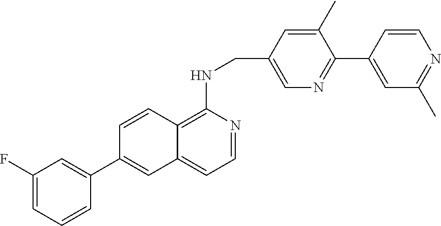 [SMILES: Cc1cc(-c2ncc(CNc3nccc4cc(-c5cccc(F)c5)ccc34)cc2C)ccn1] | MS m/z = 435.2 (M + 1); |
| 73 | 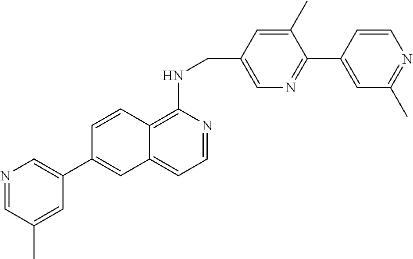 [SMILES: Cc1cncc(-c2ccc3c(NCc4cnc(-c5ccnc(C)c5)c(C)c4)nccc3c2)c1] | MS m/z = 432.2 (M + 1); |
| 74 | 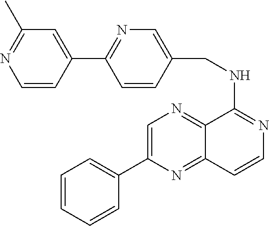 [SMILES: Cc1cc(-c2ccc(CNc3nccc4nc(-c5ccccc5)cnc34)cn2)ccn1] | MS m/z = 405.2 (M + 1); |
| 75 | 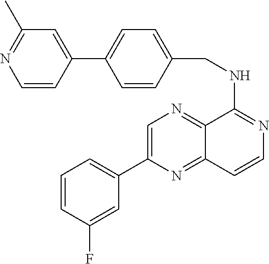 [SMILES: Cc1cc(-c2ccc(CNc3nccc4nc(-c5cccc(F)c5)cnc34)cc2)ccn1] | MS m/z = 422.2 (M + 1); |
| 76 | 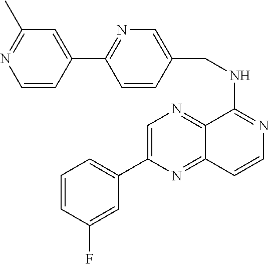 [SMILES: Cc1cc(-c2ccc(CNc3nccc4nc(-c5cccc(F)c5)cnc34)cn2)ccn1] | MS m/z = 423.2 (M + 1); |
| 77 | 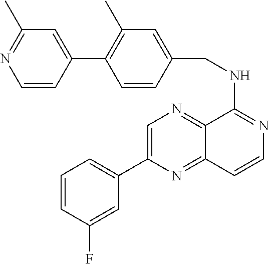 [SMILES: Cc1cc(-c2ccc(CNc3nccc4nc(-c5cccc(F)c5)cnc34)cc2C)ccn1] | MS m/z = 436.2 (M + 1); |
| 78 | 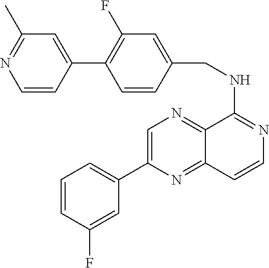 [SMILES: Cc1cc(-c2ccc(CNc3nccc4nc(-c5cccc(F)c5)cnc34)cc2F)ccn1] | MS m/z = 440.2 (M + 1); |
| 79 | 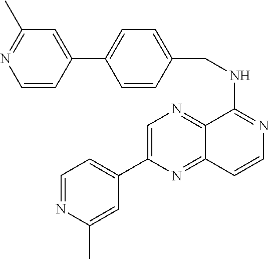 [SMILES: Cc1cc(-c2ccc(CNc3nccc4nc(-c5ccnc(C)c5)cnc34)cc2)ccn1] | MS m/z = 419.2 (M + 1); |
| 80 | 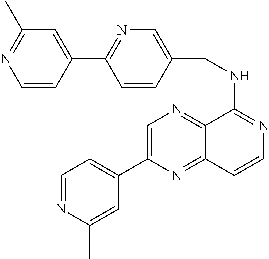 [SMILES: Cc1cc(-c2ccc(CNc3nccc4nc(-c5ccnc(C)c5)cnc34)cn2)ccn1] | MS m/z = 420.2 (M + 1); |
| 81 | 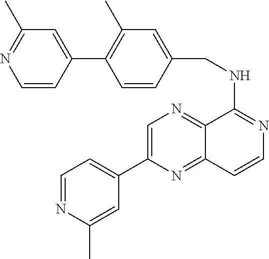 [SMILES: Cc1cc(-c2cnc3c(NCc4ccc(-c5ccnc(C)c5)c(C)c4)nccc3n2)ccn1] | MS m/z = 433.2 (M + 1); |
| 82 | 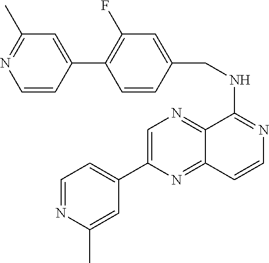 [SMILES: Cc1cc(-c2cnc3c(NCc4ccc(-c5ccnc(C)c5)c(F)c4)nccc3n2)ccn1] | MS m/z = 437.2 (M + 1); |
| 83 | 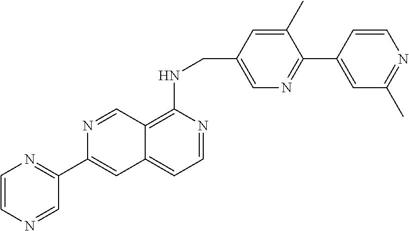 [SMILES: Cc1cc(-c2ncc(CNc3nccc4cc(-c5cnccn5)ncc34)cc2C)ccn1] | MS m/z = 420.2 (M + 1); |
| 84 | 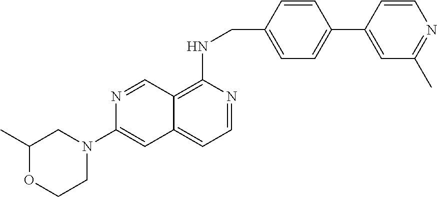 | MS m/z = 426.2 (M + 1); |
| 85 | 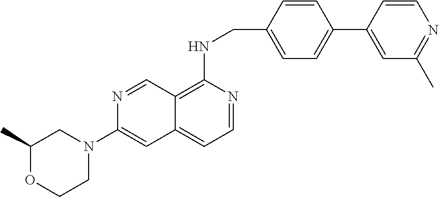 | MS m/z = 426.2 (M + 1); |
| 86 | 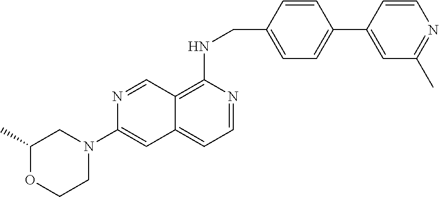 | MS m/z = 426.2 (M + 1); |
| 87 | 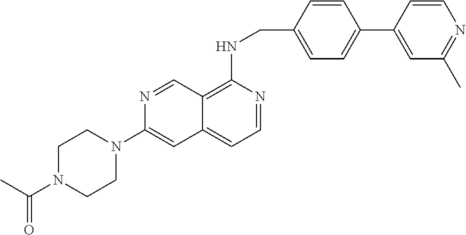 [SMILES: CC(=O)N1CCN(c2cc3ccnc(NCc4ccc(-c5ccnc(C)c5)cc4)c3cn2)CC1] | MS m/z = 453.2 (M + 1); |
| 88 | 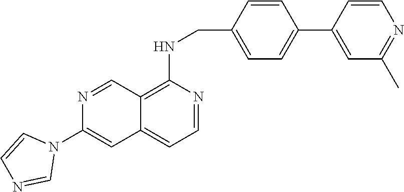 [SMILES: Cc1cc(-c2ccc(CNc3nccc4cc(-n5ccnc5)ncc34)cc2)ccn1] | MS m/z = 393.1 (M + 1); |
| 89 | 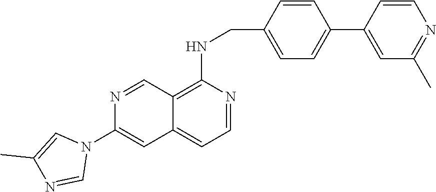 | MS m/z = 407.2 (M + 1); |
| 90 | 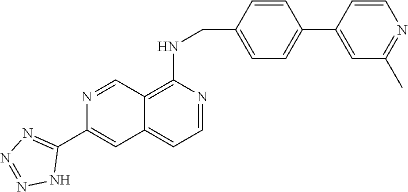 [SMILES: Cc1cc(-c2ccc(CNc3nccc4cc(-c5nnn[nH]5)ncc34)cc2)ccn1] | MS m/z = 395.1 (M + 1); |
| 91 | 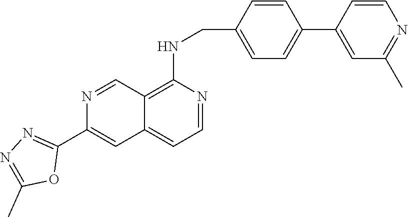 [SMILES: Cc1cc(-c2ccc(CNc3nccc4cc(-c5nnc(C)o5)ncc34)cc2)ccn1] | MS m/z = 409.2 (M + 1); |
| 92 | 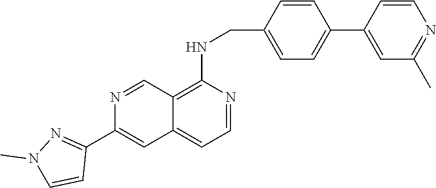 | MS m/z = 407.2 (M + 1); |
| 93 | 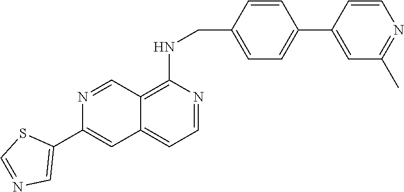 [SMILES: Cc1cc(-c2ccc(CNc3nccc4cc(-c5cncs5)ncc34)cc2)ccn1] | MS m/z = 410.2 (M + 1); |
| 94 | 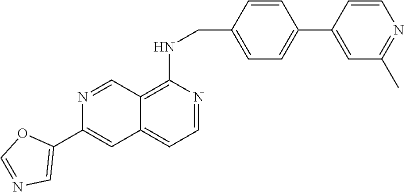 [SMILES: Cc1cc(-c2ccc(CNc3nccc4cc(-c5cnco5)ncc34)cc2)ccn1] | MS m/z = 394.1 (M + 1); |
| 95 | 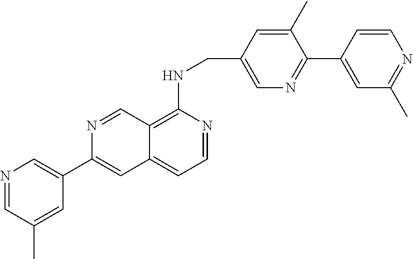 [SMILES: Cc1cncc(-c2cc3ccnc(NCc4cnc(-c5ccnc(C)c5)c(C)c4)c3cn2)c1] | MS m/z = 433.2 (M + 1); |
| 96 | 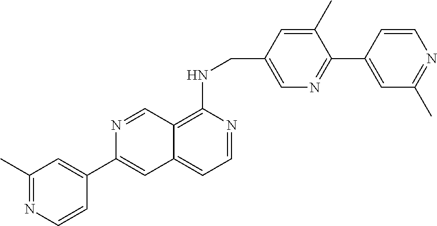 | MS m/z = 433.2 (M + 1); ¹H NMR (300 MHz, CDCl3): δ 2.30 (s, 3H), 2.55 (s, 3H), 2.61 (s, 3H), 4.86 (d, J = 5.4 Hz, 2H), 5.98 (br, 1H), 6.94 (d, J = 5.7 Hz, 1H), 7.17 (m, 1H), 7.24 (s, 1H), 7.61 (s, 1H), 7.70 (d, J = 5.1 Hz, 1H), 7.79 (s, 1H), 7.89 (s, 1H), 8.14 (d, J = 6.0 Hz, 1H), 8.49 (d, J = 5.1 Hz, 1H), 8.56 (m, 2H), 9.25 (s, 1H). |
| 97 | 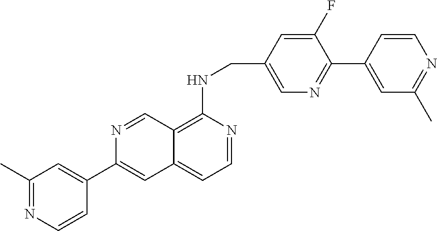 | MS m/z = 437.2 (M + 1); ¹H NMR (300 MHz, CDCl3): δ 2.31 (s, 3H), 2.61 (s, 3H), 4.90 (d, J = 5.4 Hz, 2H), 6.00 (br, 1H), 6.94 (d, J = 5.7 Hz, 1H), 7.18 (m, 1H), 7.24 (s, 1H), 7.63 (s, 1H), 7.70 (d, J = 5.1 Hz, 1H), 7.80 (s, 1H), 7.90 (s, 1H), 8.14 (d, J = 6.0 Hz, 1H), 8.33 (s, 1H), 8.50 (d, J = 5.1 Hz, 1H), 8.54 (m, 1H), 9.25 (s, 1H). |
| 98 | 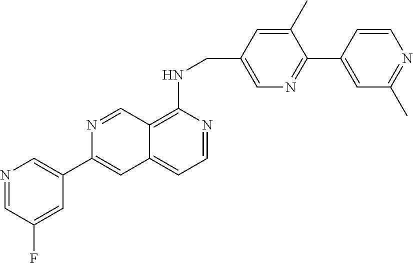 [SMILES: Cc1cc(-c2ncc(CNc3nccc4cc(-c5cncc(F)c5)ncc34)cc2C)ccn1] | MS m/z = 437.2 (M + 1); |
| 99 | 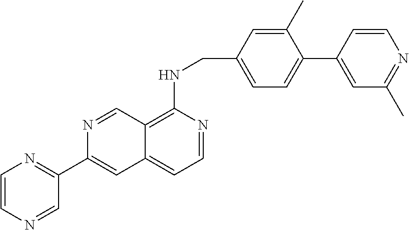 [SMILES: Cc1cc(-c2ccc(CNc3nccc4cc(-c5cnccn5)ncc34)cc2C)ccn1] | MS m/z = 419.2 (M + 1); |
| 100 | 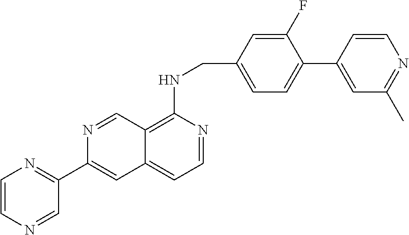 [SMILES: Cc1cc(-c2ccc(CNc3nccc4cc(-c5cnccn5)ncc34)cc2F)ccn1] | MS m/z = 423.2 (M + 1); |
| 101 | 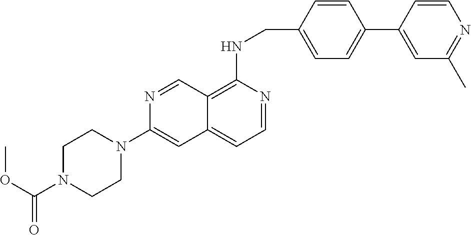 [SMILES: COC(=O)N1CCN(c2cc3ccnc(NCc4ccc(-c5ccnc(C)c5)cc4)c3cn2)CC1] | MS m/z = 469.2 (M + 1); |
| 102 | 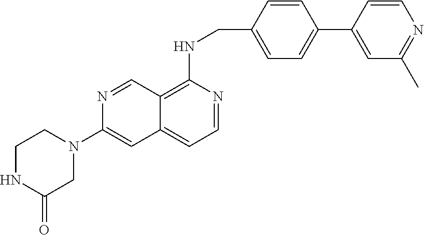 [SMILES: Cc1cc(-c2ccc(CNc3nccc4cc(N5CCNC(=O)C5)ncc34)cc2)ccn1] | MS m/z = 425.2 (M + 1); |
| 103 | 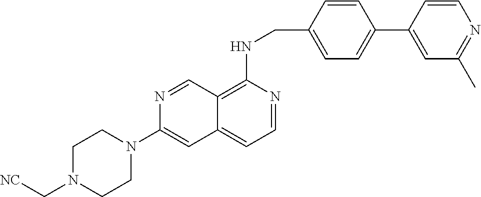 [SMILES: Cc1cc(-c2ccc(CNc3nccc4cc(N5CCN(CC#N)CC5)ncc34)cc2)ccn1] | MS m/z = 450.2 (M + 1); |
| 104 | 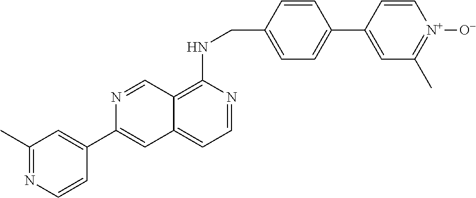 [SMILES: Cc1cc(-c2cc3ccnc(NCc4ccc(-c5cc[n+]([O-])c(C)c5)cc4)c3cn2)ccn1] | MS m/z = 434.2 (M + 1); |
| 105 | 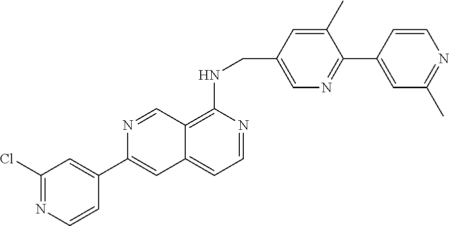 [SMILES: Cc1cc(-c2ncc(CNc3nccc4cc(-c5ccnc(Cl)c5)ncc34)cc2C)ccn1] | MS m/z = 453.2 (M + 1); |
| 106 | 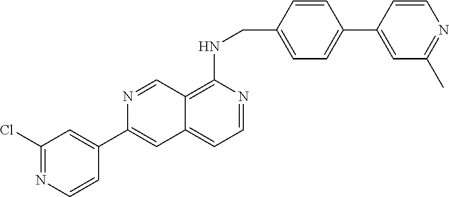 [SMILES: Cc1cc(-c2ccc(CNc3nccc4cc(-c5ccnc(Cl)c5)ncc34)cc2)ccn1] | MS m/z = 438.2 (M + 1); |
| 107 | 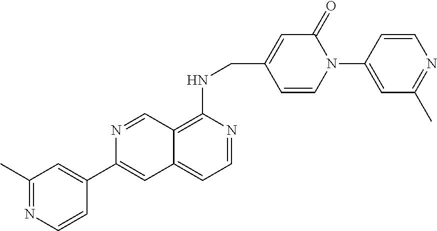 | MS m/z = 435.2 (M + 1); |
| 108 | 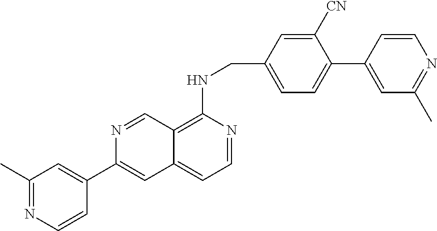 | MS m/z = 443.2 (M + 1); ¹H NMR (300 MHz, CDCl3): δ 2.30 (s, 3H), 2.61 (s, 3H), 4.98 (d, J = 5.7 Hz, 2H), 6.00 (br, 1H), 7.03 (d, J = 5.70 Hz, 1H), 7.35 (s, 1H), 7.45 (d, J = 7.8 Hz, 2H), 7.62 (s, 1H), 7.79 (d, J = 5.1 Hz, 2H), 7.89 (s, 1H), 7.98 (s, 1H), 8.20 (d, J = 5.70 Hz, 1H), 8.56 (d, J = 5.10 Hz, 2H), 8.66 (d, J = 5.10 Hz, 2H), 9.30 (s, 1H). |
| 109 | 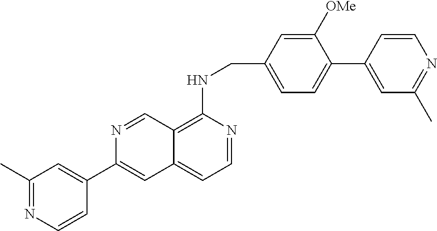 | MS m/z = 448.2 (M + 1); |
| 110 | 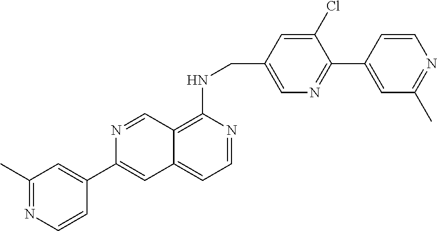 | MS m/z = 453.2 (M + 1); |
| 111 | 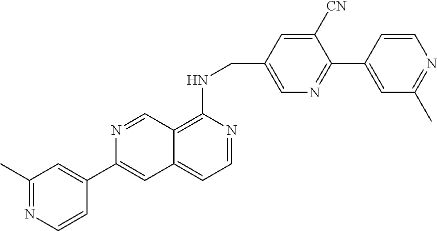 | MS m/z = 444.2 (M + 1); |
| 112 | 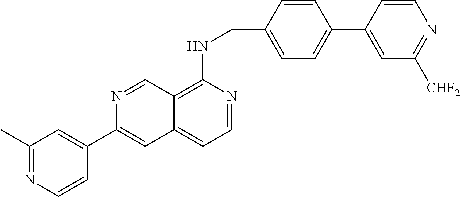 [SMILES: Cc1cc(-c2cc3ccnc(NCc4ccc(-c5ccnc(C(F)F)c5)cc4)c3cn2)ccn1] | MS m/z = 454.2 (M + 1); |

In some embodiments, the Porcupine antagonist or inhibitor used for the treatment as described herein is any suitable compound as disclosed in the WO2010/101849 A1 (PCT/US10/025813), preferably a compound of Formula II:

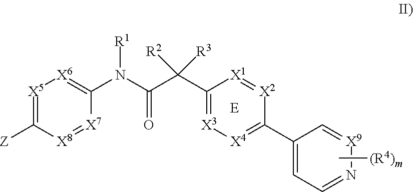

or a physiologically acceptable salt thereof, wherein:
X¹, X₂, X³ and X⁴ is selected from N and CR⁷;
one of X⁵, X₆, X⁷ and X⁸ is N and the others are CH;
X is selected from N and CH;
Z is selected from phenyl, pyrazinyl, pyridinyl, pyridazinyl and piperazinyl;
wherein each phenyl, pyrazinyl, pyridinyl, pyridazinyl or piperazinyl of Z is optionally substituted with an R⁶ group;
R¹, R² and R³ are hydrogen;
m is 1;
R⁴ is selected from hydrogen, halo, difluoromethyl, trifluoromethyl and methyl;
R⁶ is selected from hydrogen, halo and —C(O)Rⁱ⁰; wherein R¹⁰ is methyl; and
R⁷ is selected from hydrogen, halo, cyano, methyl and trifluoromethyl.

In some embodiments, the compound is selected from the group consisting of:
N-[5-(3-fluorophenyl)pyridin-2-yl]-2-[5-methyl-6-(pyridazin-4-yl)pyridin-3-yl]acetamide;
2-[5-methyl-6-(2-methylpyridin-4-yl)pyridin-3-yl]-N-[5-(pyrazin-2-yl)pyridin-2-yl]acetamide (LGK974);
N-(2,3′-bipyridin-6′-yl)-2-(2′,3-dimethyl-2,4′-bipyridin-5-yl)acetamide;
N-(5-(4-acetylpiperazin-1-yl)pyridin-2-yl)-2-(2′-methyl-3-(trifluoromethyl)-2,4′-bipyridin-5-yl)acetamide;
N-(5-(4-acetylpiperazin-1-yl)pyridin-2-yl)-2-(2′-fluoro-3-methyl-2,4′-bipyridin-5-yl)acetamide; and
2-(2′-fluoro-3-methyl-2,4′-bipyridin-5-yl)-N-(5-(pyrazin-2-yl)pyridin-2-yl)acetamide; or a pharmaceutically acceptable salt thereof.

In some embodiments, the compound is 2-[5-methyl-6-(2-methylpyridin-4-yl)pyridin-3-yl]-N-[5-(pyrazin-2-yl)pyridin-2-yl]acetamide.

### III. Medical and Pharmaceutical Uses

Compounds of the invention are indicated as pharmaceuticals. According to a further aspect of the invention there is provided a compound of the invention, as hereinbefore (but without any provisos, where applicable), for use as a pharmaceutical. There is also provided a synthetic form of a compound of the invention (but without any provisos, where applicable), for use as a pharmaceutical.

For the avoidance of doubt, although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolized in the body to form compounds of the invention. Such compounds (which may possess some pharmacological activity, provided that such activity is appreciably lower than that of the “active” compounds to which they are metabolized) may therefore be described as “prodrugs” of compounds of the invention.

By“prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention (as hereinbefore defined but without the proviso(s)) may be useful in the treatment of a cancer. By “cancer”, we mean any disease that arises from an uncontrolled growth of cells (e.g. uncontrolled division), invasion (e.g. direct growth into adjacent tissue) or metastasis. By “uncontrolled growth”, we include an increase in the number and/or size of cancer cells (also referred to herein as “proliferation”). By “metastasis” we mean the movement or migration (e.g. invasiveness) of cancer cells from a primary tumor site in the body of a subject to one or more other areas within the subject's body (where the cells can then form secondary tumors). Thus, in one embodiment the invention provides compounds and methods for inhibiting, in whole or in part, the formation of secondary tumors in a subject with cancer.

Advantageously, the compounds of the invention may be capable of inhibiting the proliferation and/or metastasis of cancer cells selectively.

By “selectively” we mean that the compounds of the invention may inhibit the proliferation and/or metastasis of cancer cells to a greater extent than it modulates the function (e.g. proliferation) of non-cancer cells. Preferably, the compounds of the invention inhibit the proliferation and/or metastasis of cancer cells only.

In another aspect, the present invention provides a pharmaceutical composition comprising the compound of the present invention and at least one pharmaceutically acceptable carrier or diluent, wherein said compound is in free form or in a pharmaceutically acceptable salt form. Such composition may be an oral composition, injectable composition or suppository. And the composition may be manufactured in a conventional manner by mixing, granulating or coating methods.

In an embodiment of the invention, the composition is an oral composition and it may be a tablet or gelatin capsule. Preferably, the oral composition comprises the present compound together with a) diluents, e.g., lactose, dextrose, sucrose, mannitol, sorbitol, cellulose and/or glycine; b) lubricants, e.g., silica, talcum, stearic acid, its magnesium or calcium sat and/or polyethyleneglycol; for tablets, together with c) binders, e.g., magnesium aluminum silicate, starch paste, gelatin, tragamayth, methylcellulose, sodium carboxyl)methylcellulose and or polyvinylpyrrolidone; and if desired, d) disintegrants, e.g., starches, agar, alginic acid or its sodium salt, or effervescent mixtures; and/or e) additives, e.g., absorbents, colorants, flavors and sweeteners.

In another embodiment of the invention, the composition is an injectable composition, and may be an aqueous isotonic solution or suspension.

In yet another embodiment of the invention, the composition is a suppository and may be prepared from fatty emulsion or suspension.

Preferably, the composition is sterilized and/or contains adjuvant. Such adjuvant can be preserving, stabilizing, wetting or emulsifying agent, solution promoter, salt for regulating the osmotic pressure, buffer and/or any combination thereof.

Alternatively or in addition, the composition may further contain other therapeutically valuable substances for different applications, like solubilizers, stabilizers, tonicity enhancing agents, buffers and/or preservatives.

In an embodiment of the invention, the composition may be a formulation suitable for transdermal application. Such formulation includes an effective amount of the compound of the present invention and a carrier. Preferably, the carrier may include absorbable pharmacologically acceptable solvents to assist passage through the skin of the host. A transdermal device contain the formulation may also be used. The transdermal device may be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Otherwise, a matrix transdermal formulation may also be used.

In another embodiment of the invention, the composition may be a formulation suitable for topical application, such as to the skin and eyes, and may be aqueous solution, ointment, cream or gel well known in the art.

In another aspect, the present invention provides a method of inhibiting WNT secretion from a cell.

In one embodiment, the cell is contained within a mammal, and the administered amount is a therapeutically effective amount. In another embodiment, the inhibition of WNT signaling further results in the inhibition of the growth of the cell. In a further embodiment, the cell is a cancer cell. In yet another embodiment, the cell is a fibrogenic cell.

Cell proliferation is measured by using methods known to those skilled in the art. For example, a convenient assay for measuring cell proliferation is the CellTiter-Glo™ Assay commercially available from Promega (Madison, Wis.). The assay procedure involves adding the CellTiter-Glo® reagent to cells cultured on multi-well dishes. The luminescent signal, measured by a luminometer or an imaging device, is proportional to the amount of ATP present, which is directly proportional to the number of viable cells present in culture. In addition, cell proliferation may also be measured using colony formation assays known in the art.

The present invention also provides a method for treating cancers or fibroses related to the WNT signaling pathway with an effective amount of the present compound. Those skilled in the art would readily be able to determine whether a cancer is related to the Wnt pathway by analyzing cancer cells using one of several techniques known in the art. For example, one could examine cancer cells for aberrations in the levels of proteins or mRNAs involved in Wnt signaling using immune and nucleic acid detection methods.

Cancers or fibroses related to the Wnt pathway include those in which activity of one or more components of the Wnt signaling pathways are upregulated from basal levels. In one embodiment, inhibiting the Wnt pathway may involve inhibiting Wnt secretion. As another example, inhibiting the Wnt pathway may involve inhibiting components downstream of the cell surface receptors. In another embodiment, inhibition of Wnt secretion may involve inhibiting the activity of any of the proteins implicated in the secretion of functional WNTs.

Furthermore, the invention provides a method for treating a WNT pathway disorder in a subject suffering from the disorder by administering to the subject a therapeutically effective amount of a WNT inhibitor. In one embodiment, the disorder is a cell proliferative disorder associated with aberrant, e.g., increased, activity of WNT signaling. In another embodiment, the disorder results from increased amount of a WNT protein. In yet another embodiment, the cell proliferative disorder is cancer, include but are not limited to: lung (small cell and non-small cell), breast, prostate, carcinoid, bladder, gastric, pancreatic, liver (hepatocellular), hepatoblastoma, colorectal, head cancer and neck squamous cell carcinoma, esophageal, ovarian, cervical, endometrial, mesothelioma, melanoma, sarcoma, osteosarcoma, liposarcoma, thyroid, desmoids, chronic myelocytic leukemia (AML), and chronic myelocytic leukemia (CML). In yet another embodiment, the cell proliferative disorder is fibrosis, include but are not limited to: lung fibrosis, such as idiopathic pulmonary fibrosis and radiation-induced fibrosis, renal fibrosis and liver fibrosis including liver cirrhosis. In yet another embodiment, the disorder is osteoarthritis, Parkinson's disease, retinopathy, macular degeneration.

For therapeutically use, the compound of the present invention could be administered in a therapeutically effective amount via any acceptable way known in the art singly. As used herein, the therapeutically effective amount may vary widely depending on the severity of the disease, the age and relative health of the subject, the potency of the compound used and other factors. Generally, the satisfactory result is indicated to be obtained systemically at a daily dosage of about 0.03 to 2.5 mg/kg per body weight of the subject. In one embodiment, the indicated daily dosage for larger mammal as human is in the range from about 0.5 mg to about 100 mg. Preferably, the compound is administered in divided doses up to four times a day or in retard form. In another embodiment, suitable unit dosage forms for oral administration comprise from ca. 1 to 100 mg active ingredient.

Alternatively, the compound of the present invention may be administered in a therapeutically effective amount as the active ingredient in combination with one or more therapeutic agents, such as pharmaceutical combinations. There may be synergistic effects when the compound of the present invention is used with a chemotherapeutic agent known in the art. The dosage of the co-administered compounds could vary depending on the type of co-drug employed, the specific drug employed, the condition being treated and so forth.

The compound of the present invention or the composition thereof may be administered by any conventional route. In one embodiment, it is administered enterally, such as orally, and in the form of tablets or capsules. In another embodiment, it is administered parenterally and in the form of injectable solutions or suspensions. In yet another embodiment, it is administered topically and in the form of lotions, gels, ointments or creams, or in a nasal or suppository form.

In another aspect, the invention also provides a pharmaceutical combination, preferably, a kit, comprising a) a first agent which is the compound of the present invention as disclosed herein, in free form or in pharmaceutically acceptable sat form, and b) at least one co-agent. In addition, the kit may comprise instructions for its administration.

The combination of the present invention may be used in vitro or in vivo. Preferably, the desired therapeutic benefit of the administration may be achieved by contacting cell, tissue or organism with a single composition or pharmacological formulation that includes the compound of the present invention and one or more agents, or by contacting the cell with two or more distinct compositions or formulations, wherein one composition includes one agent and the other includes another. The agents of the combination may be administered at the same time or separately within a period of time. Preferably, the separate administration can result in a desired therapeutic benefit. The present compound may precede, be co-current with and/or follow the other agents by intervals ranging from minutes to weeks. A person skilled in the art could generally ensure the interval of the time of each delivery, wherein the agents administered separately could still be able to exert an advantageously combined effect on the cell, tissue or organism. In one embodiment, it is contemplated that one may contact the cell, tissue or organism with two, three, four or more modalities substantially simultaneously as the candidate substance, i.e., with less than about one minute. In another embodiment, one or more agents may be administered about between 1 minute to 14 days.

In another aspect, the present provides a process for preparing the compound of the present invention or the salts or derivatives thereof.

In one embodiment, the compound having Formula (1) may be prepared following any one of the synthetic methodologies described in Examples below. In the reactions described, reactive functional groups, for example hydroxy, amino, imino, thio or carboxy groups, where these are desired in the final product, may be protected to avoid their unwanted participation in the reactions. Conventional protecting groups may be used in accordance with standard practice (see e.g., T. W. Greene and P. G. M. Wuts in “Protective Groups in Organic Chemistry”, John Wiley and Sons, 1991). Suitable leaving groups for use in the synthetic methodologies described include halogen leaving groups and other conventional leaving groups known in the art. Preferably, the leaving group is chloro or bromo.

In another embodiment, the compound of the invention or the salts thereof may also be obtainable in the form of hydrates, or their crystals may include for example the solvent used for crystallization (present as solvates). Salts can usually be converted to compounds in free form by treating with suitable basic agents, preferably with alkali metal carbonates, alkali metal hydrogen carbonates, or alkali metal hydroxides, more preferably with potassium carbonate or sodium hydroxide. A compound of the invention in a base addition salt form may be converted to the corresponding free acid by treating with a suitable acid, such as hydrochloric acid. In view of the close relationship between the novel compounds in free form and those in the form of their salts, including those salts that may be used as intermediates, for example in the purification or identification of the novel compounds, any reference to the free compounds is to be understood as referring also to the corresponding salts, as appropriate.

Salts of the present compound with a salt-forming group may be prepared in a manner known in the art. Acid addition salts of compound of Formula (1) may thus be obtained by treatment with an acid or with a suitable anion exchange reagent. Pharmaceutically acceptable salts of the compound of the invention may be formed as acid addition salts from compound of Formula (1) with a basic nitrogen atom with organic or inorganic acids.

Preferably, suitable inorganic acids include, but are not limited to, halogen acids, such as hydrochloric acid, sulfuric acid, or phosphoric acid.

Preferably, suitable organic acids include, but are not limited to, carboxylic, phosphoric, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, -malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4 aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4 methylbenzenesulfonic acid, methylsulfuric acid, ethylsulfuric acid, dodecylsulfuric acid, N cyclohexylsulfamic acid, N-methyl-, N-ethyl- or N-propyl-sulfamic acid, or other organic protonic acids, such as ascorbic acid.

Alternatively, it is also possible to use pharmaceutically unacceptable salts for isolation or purification, for example picrates or perchlorates. But for therapeutic use, only pharmaceutically acceptable salts or free compounds are employed, where applicable in the form of pharmaceutical preparations.

In yet another embodiment, compound of the present invention in unoxidized form may be prepared from N-oxides of compound of the invention by treating with a reducing agent in a suitable inert organic solvent at 0 to 80° C. Preferably, the reducing agent is sulfur, sulfur dioxide, triphenyl phosphine, lithium borohydride, sodium borohydride, phosphorus trichloride, tribromide, or the like. Preferably, the invert organic solvent is acetonitrile, ethanol, aqueous dioxane, or the like.

In yet another embodiment, prodrug derivatives of the compound of the present invention may be prepared by methods known in the art (for further details see Saulnier at al., (1994), Bioorganic and Medicinal Chemistry Letters, Vol. 4, p. 1985). In a preferable embodiment, an appropriate prodrug may be prepared by reacting a non-derivatized compound of the invention with a suitable carbamylating agent such as 1,1-acyloxyalkylcarbanochloridate, para-nitrophenyl carbonate, or the like.

In yet another embodiment, protected derivatives of the compound of the present invention may be made by means known in the art. A detailed description of techniques applicable to the creation of protecting groups and their removal may be found in T. W. Greene, “Protecting Groups in Organic Chemistry”, 3rd edition, John Wiley and Sons, Inc., 1999.

In yet another embodiment, compound of the present invention may be prepared as their individual stereoisomers. The process includes reacting a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compound of the present invention, or by using dissociable complexes such as crystalline diastereomeric salts. Diastereomers have distinct physical properties presented by melting points, boiling points, solubilities, reactivity, etc., and may be readily separated by taking advantage of these dissimilarities. The diastereomers may be separated by fractionated crystallization, chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture may be found in Jean Jacques, Andre Collet, Samuel H. Wilen, “Enantiomers, Racemates and Resolutions”, John Wiley And Sons, Inc., 1981.

In conclusion, the compound of the present invention could be made by the process described in the Examples; optionally a pharmaceutically acceptable salt may be converted from the compound of the present invention; optionally a pharmaceutically acceptable N-oxide may be converted from an unoxidized form of the compound the present invention; optionally an individual isomer of the compound of the present invention is resolved from a mixture of isomers; and optionally a pharmaceutically acceptable prodrug derivative may be converted from a non-derivatized compound of the present invention.

Insofar as the production of the starting materials is not particularly described, the compounds are known or can be prepared analogously to methods known in the art or as disclosed in the Examples hereinafter. One of skill in the art will appreciate that the above transformations are only representative of methods for preparation of the compounds of the present invention, and that other well-known methods can similarly be used.

### IV. Patent Selection and Treatment of Cancer

In another aspect, the present invention provides compositions and methods for treatment of cancer characterized by overexpression of R-spondin and/or expression of an R-spondin fusion in a subject that has been diagnosed as having overexpression of R-spondin and/or R-spondin fusion and is in need of such treatment.

R-spondins (RSPOs) are a family of four cysteine-rich secreted proteins containing a single thrombospondin type I repeat (TSR) domain. The Rspo gene family is evolutionary conserved and can be found in the genomic and transcript databases of all deuterostomes including the hemichordate, Saccoglossus kowalevskii (acorn worm), the chordate, Ciona intestinalis (tunicate), and the echinoderm. RSPOs from different vertebrate species display the properties of the canonical WNT signaling activators. The CR domain of the RSPO proteins is primarily responsible for mediating the activation of the WNT/β-catenin signaling pathway. The TSR and BR domains are proposed to regulate the strength of RSPO activity on canonical WNT signaling, because the RSPO protein lacking the TSR and BR domains activates canonical WNT signaling less effectively. Yoon, J. K. & Lee, J. S. Cellular signaling and biological functions of R-spondins. Cell. Signal. 24, 369-377 (2012).

By “R-spondin fusion” herein is meant a fusion between one of the Rspo genes (including but not limited to Rspo2 and Rspo3 genes) and another gene (“Fusion partner gene”), including, but not limited to PTPRK, EIF3E, EMC2, PVT1, and HNF4G genes. The fusion may be due to deletion or inversion. The fusion of Rspo gene to the 5′partner gene generally leads to expression of Rspo gene (full length or partial as part of the fusion gene product) under the control of a promoter of a different gene (e.g. the fusion partner gene), which leads to change of expression level (e.g., elevated expression) of Rspo gene (e.g., a fusion gene) at the mRNA level and/or protein level. The Rspo fusion gene may produe to a functional or non-functional Rspo fragment.

“Characterized by” with respect to a cancer and mutant R-spondin polynucleotide and polypeptide is meant a cancer in which a gene deletion or translocation and/or expressed fusion polypeptide involving R-spondin are present as compared to a cancer in which such gene deletion and/or fusion polypeptide are not present. The presence of mutant polypeptide may drive, in whole or in part, the growth and survival of such cancer.

The compositions provided herein are used to treat a variety of cancers that involve Rspo fusion, such as colorectal cancer, gastric cancer, liver cancer, esophageal cancer, intestinal cancer, bile duct cancer, pancreatic cancer, endometrial cancer, and prostate cancer.

A mechanism for certain tumors, such as colorectal tumors and prostate tumors, to gain activation of the WNT pathway is that two genes encoding enhancers of WNT ligands, R spondin-2 and R spondin-3, are transcriptionally activated by fusion to other genes, such as PTPRK, EIF3E, EMC2, PVT1, and HNF4G genes. See Examples provided herein, Seshagiri S, et al. Recurrent R-spondin fusions in colon cancer. Nature. 2012 Aug. 30; 488(7413):660-4, and Robinson et al, Integrative Clinical Genomics of Advanced Prostate Cancer, Cell 161, 1215-1228 May 21, 2015, which are incorporated by reference in their entirety. The Rsop fusion gene may lead to a functional or non-functional Rspo protein fragment. When a functional Rspo protein is generated, it may act as an activator of Wnt pathway, which may cause the proliferation of tumor cells.

The present invention provides methods and compositions for screening for cancer patients with Rspo fusions using methods known in the art and/or provided herein, and optionally treating such patients with Wnt inhibitor as provided herein.

The Rspo gene can be detected at genomic DNA level, mRNA level, or protein level. A biological sample from a subject in need of testing is obtained using methods known the art. The biological sample is optionally processed to obtain protein, RNA, and/or DNA, which is in turn used in assays to detect Rspo fusion.

### A. Biological Sample

By “biological sample” herein is meant any biological sample suspected of containing Rspo fusion polynucleotides or polypeptides or fragments thereof (including Rspo-PTPRK and Rspo-EIF3E fusion polynucleotides and polypeptides), and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells, blood, urine, marrow, or a tissue, and the like.

Biological samples useful in the practice of the methods of the invention may be obtained from any mammal in which a cancer characterized by the expression of an Rspo3-PTPRK or Rspo2-EIF3E fusion polypeptide is present or developing. In one embodiment, the mammal is a human, and the human may be a candidate for a Wnt-inhibiting therapeutic for the treatment of a cancer, e.g. colon, gastric and esophageal cancer. The human candidate may be a patient currently being treated with, or considered for treatment with, a Wnt inhibitor, such as those provided herein. In another embodiment, the mammal is large animal, such as a horse or cow, while in other embodiments, the mammal is a small animal, such as a dog or cat, all of which are known to develop cancers, including colon, gastric and esophageal carcinomas.

Any biological sample comprising cells (or extracts of cells) from a mammalian cancer is suitable for use in the methods of the invention. Circulating tumor cells may also be obtained from serum using tumor markers, cytokeratin protein markers or other methods of negative selection as described (see Ma et al., Anticancer Res. 23 (1A): 49-62 (2003)). Serum and bone marrow samples may be particularly preferred for patients with leukemia. For cancers involving solid tumors, such as sarcomas and carcinomas, the biological sample may comprise cells obtained from a tumor biopsy, which may be be obtained according to standard clinical techniques.

Circulating tumor cells (“CTCs”) may be purified, for example, using the kits and reagents sold under the trademarks Vita-Assays™, Vita-Cap™, and Cell Search® (commercially available from Vitatex, LLC (a Johnson and Johnson corporation). Other methods for isolating CTCs are described (see, for example, PCT Publication No. WO/2002/020825, Cristofanilli et al., New Engl. J. of Med. 351 (8):781-791 (2004), and Adams et al., J. Amer. Chem. Soc. 130(27): 8633-8641 (July 2008)). In a particular embodiment, a circulating tumor cell (“CTC”) may be isolated and identified as having originated from the lung, or colon, stomach, esophagus.

### B. Detection of Rspo Fusion Polypeptide

In some embodiments, the Rspo fusion is detected by an immunoassay. An Rspo fusion protein or peptide is generated to produce antibodies (monoclonal or polyclonal) specific for Rspo fusion proteins. Such antibodies are then used in an assay to detect the presence of Rspo fusion.

Rspo fusion is generally detected using a Rspo fusion-specific reagent. By “Rspo fusion polypeptide-specific reagent” herein is meant any reagent, biological or chemical, capable of specifically binding to, detecting and/or quantifying the presence/level of expressed Rspo fusion polypeptide in a biological sample. The term includes, but is not limited to, the preferred antibody and reagents discussed below, and equivalent reagents are within the scope of the present invention.

Reagents suitable for use in practice of the methods of the invention include an PTPRK-Rspo3 fusion polypeptide-specific antibody and/or EIF3E-Rspo2 fusion polypeptide-specific antibody, or other Rspo2 or Rspo3 fusion proteins as provided herein. A fusion-specific antibody of the invention is an isolated antibody or antibodies that specifically bind(s) an PTPRK-Rspo3 fusion polypeptide of the invention (e.g. the peptide corresponding to the PTPRK-Rspo3 fusion sequences provided herein, or other Rspo2 or Rspo3 fusion proteins as provided herein) but does not substantially bind either wild type Rspo or wild type PTPRK, or specifically bind(s) a EIF3E-Rspo2 fusion polypeptide described herein (e.g. the peptide corresponding to the Rspo2-EIF3E fusion sequences provided herein) but does not substantially bind either wild type Rspo or wild type EIF3E.

Human PTPRK-Rspo3 or EIF3E-Rspo2 fusion polypeptide (or other Rspo2 or Rspo3 fusion proteins as provided herein)-specific antibodies may also bind to highly homologous and equivalent epitopic peptide sequences in other mammalian species, for example murine or rabbit, and vice versa. Antibodies useful in practicing the methods of the invention include (a) monoclonal antibodies, (b) purified polyclonal antibodies that specifically bind to the target polypeptide (e.g. the fusion junction of Rspo3-PTPRK fusion polypeptide or Rspo2-EIF3E fusion polypeptide or other Rspo2 or Rspo3 fusion proteins as provided herein, (c) antibodies as described in (a)-(b) above that bind equivalent and highly homologous epitopes or phosphorylation sites in other non-human species (e.g. mouse, rat), and (d) fragments of (a)-(c) above that bind to the antigen (or more preferably the epitope) bound by the exemplary antibodies disclosed herein

By “antibody” or “antibodies” herein is meant all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.)

The invention is not limited to use of antibodies, but includes equivalent molecules, such as protein binding domains or nucleic acid aptamers, which bind, in a fusion-protein or truncated-protein specific manner, to essentially the same epitope to which an Rspo3-PTPRK or Rspo2-EIF3E fusion polypeptide-specific antibody useful in the methods of the invention binds. See, e.g., Neuberger at al., Nature 312: 604 (1984). Such equivalent non-antibody reagents may be suitably employed in the methods of the invention further described below.

Polyclonal antibodies useful in practicing the methods of the invention may be produced according to standard techniques by immunizing a suitable animal (e.g., rabbit, goat, etc.) with an antigen encompassing a desired fusion-protein specific epitope (e.g. the fusion junction of an Rspo fusion protein described herein), collecting immune serum from the animal, and separating the polyclonal antibodies from the immune serum, and purifying polyclonal antibodies having the desired specificity, in accordance with known procedures. The antigen may be a synthetic peptide antigen comprising the desired epitopic sequence, selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)). Polyclonal antibodies produced as described herein may be screened and isolated as further described below.

Monoclonal antibodies may also be beneficially employed in the methods of the invention, and may be produced in hybridoma cell lines according to the well-known technique of Kohler and Milstein. Nature 265: 495-97 (1975); Kohler and Milstein, Eur. J. Immunol. 6:511 (1976); see also, CURRENT PROTOCOLS IN MOLECULAR BIOLOGY, Ausubel et al. Eds. (1989). Monoclonal antibodies so produced are highly specific, and improve the selectivity and specificity of assay methods provided by the invention. For example, a solution containing the appropriate antigen (e.g. a synthetic peptide comprising the fusion junction of Rspo3-PTPRK or Rspo2-EIF3E fusion polypeptide) may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, K. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Aced. Sci., 82: 8653 (1985); Spira et al., J. Immunol. Methods, 74: 307 (1984)). The antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Further still, U.S. Pat. No. 5,194,392, Geysen (1990) describes a general method of detecting or determining the sequence of monomers (amino acids or other compounds) that is a topological equivalent of the epitope (i.e., a “mimotope”) that is complementary to a particular paratope (antigen binding site) of an antibody of interest. More generally, this method involves detecting or determining a sequence of monomers that is a topographical equivalent of a ligand that is complementary to the ligand binding site of a particular receptor of interest. Similarly, U.S. Pat. No. 5,480,971, Houghten et al. (1996) discloses linear C₁-C-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Antibodies useful in the methods of the invention, whether polyclonal or monoclonal, may be screened for epitope and fusion protein specificity according to standard techniques. See, e.g. Czernik et al., Methods in Enzymology, 201: 264-283 (1991). For example, the antibodies may be screened against a peptide library by ELISA to ensure specificity for both the desired antigen and, if desired, for reactivity only with, e.g. an Rspo3-PTPRK fusion polypeptide of the invention and not with wild-type Rspo3 or wild-type PTPRK. The antibodies may also be tested by Western blotting against cell preparations containing target protein to confirm reactivity with the only the desired target and to ensure no appreciable binding to other fusion proteins involving Rspo. The production, screening, and use of fusion protein-specific antibodies is known to those of skill in the art, and has been described. See, e.g., U.S. Patent Publication No. 20050214301, Wetzel et al., Sep. 29, 2005.

Fusion polypeptide-specific antibodies useful in the methods of the invention may exhibit some limited cross-reactivity with similar fusion epitopes in other fusion proteins or with the epitopes in wild type Rspo, wild type PTPRK, and wild type EIF3E that form the fusion junction. This is not unexpected as most antibodies exhibit some degree of cross-reactivity, and anti-peptide antibodies will often cross-react with epitopes having high homology or identity to the immunizing peptide. See, e.g., Czernik, supra. Cross-reactvty with other fusion proteins is readily characterized by Western blotting alongside markers of known molecular weight. Amino acid sequences of cross-reacting proteins may be examined to identify sites highly homologous or identical to the Rspo3-PTPRK or Rspo2-EIF3E fusion polypeptide sequence to which the antibody binds. Undesirable cross-reactivity can be removed by negative selection using antibody purification on peptide columns (e.g. selecting out antibodies that bind either wild type Rspo, wild type PTPRK, and/or wild type EIF3E).

Rspo3-PTPRK or Rspo2-EIF3E fusion polypeptide specific antibodies of the invention that are useful in practicing the methods disclosed herein are ideally specific for human fusion polypeptide, but are not limited only to binding the human species, per se. The invention includes the production and use of antibodies that also bind conserved and highly homologous or identical epitopes in other mammalian species (e.g. mouse, rat, monkey). Highly homologous or identical sequences in other species can readily be identified by standard sequence comparisons, such as using BLAST, with a human Rspo3-PTPRK or Rspo2-EIF3E fusion polypeptide.

Antibodies employed in the methods of the invention may be further characterized by, and validated for, use in a particular assay format, for example flow cytometry (FC), immunohistochemistry (IHC), and/or Immunocytochemistry (ICC). Antibodies may also be advantageously conjugated to fluorescent dyes (e.g. Alexa488, PE), or labels such as quantum dots, for use in multi-parametric analyses along with other signal transduction (phospho-AKT, phospho-Erk 1/2) and/or cell marker (cytokeratin) antibodies.

### C. Detection of Rspo Fusion Polynucleotide

Fusion-specific reagents provided by the invention also include nucleic acid probes and primers suitable for detection of an Rspo3-PTPRK or Rspo2-EIF3E fusion polynucleotide, or other Rspo2 or Rspo3 fusion polynucleotides, as provided herein. Such probes desirably include, among others, breakpoint probes corresponding to both sides of the breakpoints in wild-type Rspo and/or wildtype PTPRK genes, or wild-type Rspo and/or wild-type EIF3E genes, that produce the fusion. Specific use of such probes in assays such as fluorescence in-situ hybridization (FISH) or polymerase chain reaction (PCR) amplification is described herein.

In some embodiments, the Rspo fusion is detected by PCR, such as regular PCR, Real-time PCR (Q-PCR) or digital PCR. A pair of primers is used to amplify the fusion genes. The primers are designed based on the fusion gene sequence to be amplified. Preferably, one primer hybridizes to a first sequence of an Rspo gene and the second primer hybridizes to a second sequence of a fusion partner gene. PCR can be performed on either cDNA (as prepared from RNA using the biological sample) or genomic DNA, under conditions that can be optimized as known in the art.

In some embodiments, FISH is employed (as described in Verma et al. HUMAN CHROMOSOMES: A MANUAL OF BASIC TECHNIQUES, Pergamon Press, New York, N.Y. (1988)) and may be correlated with other physical chromosome mapping techniques and genetic map data. Examples of genetic map data can be found in the 1994 Genome Issue of Science (265: 1981). Correlation between the location of the gene encoding Rspo3-PTPRK or Rspo2-EIF3E fusion polypeptide on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In some embodiments, a first probe hybridizes to an Rspo gene sequence and is labeled with a first color (e.g., red) and a second probe hybridizes to a fusion partner gene sequence and is labeled with a second color (e.g., green). In the case of Rspo fusion, the two probes hybridize to the fusion gene and become adjacent to each other. As a result, the images of the two probes will merger, which results in a different color (e.g., yellow).

It shall be understood that all of the methods (e.g., PCR and FISH) that detect Rspo3-PTPRK or Rspo2-EIF3E fusion polynucleotides of the invention may be combined with other methods that detect either mutant Rspo polynucleotides or mutant Rspo polypeptides. For example, detection of a Rspo3-PTPRK or Rspo2-EIF3E fusion polynucleotide in the genetic material of a biological sample (e.g., in a circulating tumor cell) may be followed by Western blotting analysis or immuno-histochemistry (IHC) analysis of the proteins of the sample to determine if the Rspo3-PTPRK or Rspo2-EIF3E fusion polynucleotide was actually expressed as a Rspo3-PTPRK or Rspo2-EIF3E fusion polypeptide in the biological sample. Such Western blotting or IHC analyses may be performed using an antibody that specifically binds to the polypeptide encoded by the detected Rspo3-PTPRK or Rspo2-EIF3E fusion polynucleotide, or the analyses may be performed using antibodies that specifically bind either to full length Rspo (e.g., bind to the N-terminus of the protein) or to full length PTPRK (e.g., bind an epitope in the kinase domain of PTPRK). Such assays are known in the art (see, e.g., U.S. Pat. No. 7,488,252).

In another example, the CISH technology of Dako allows chromatogenic in-situ hybridization with immuno-histochemistry on the same tissue section.

In some embodiments, the Rspo fusion is detected by hybridization in a Southern blot assay using a probe that comprise sequences from both the Rspo gene and the fusion partner gene.

In some embodiments, the Rspo fusion is detected by other hybridization-based methods, such as microarray, branched DNA (QuantiGene®), ViewRNA® or RNAscope®.

In some embodiments, the Rspo fusion is detected by hybridization using microarray where a custom fusion gene microarray is used to detect Rspo fusion transcripts from cancer specimens. The oligos are designed to enable combined measurements of chimeric transcript junctions with exon-wise measurements of individual fusion partners. See Skotheim, R I; Thomassen, G O; Eken, M; Lind, G E; Micci, F; Ribeiro, F R; Cerveira, N; Teixeira, M R et al. A universal assay for detection of oncogenic fusion transcripts by oligo microarray analysis. Molecular Cancer 8: 5. (2009).

In some embodiments, the Rspo fusion is detected by hybridization using branched DNA assay. In these embodiments a custom hybridization and signal amplification assay, such as the branched DNA assay (QuantiGene®), is used to detect Rspo fusion transcripts in lysis solutions from cancer specimens. The sequences of capture extender probes and the label extender probes are derived from the exon sequences of Rspo genes and fusion partner genes (e.g., PTPRK for Rspo3, EIF3E for Rspo2) such as those exemplified in Example 9. See, Lu B., et al. Detection of TMPRSS2-ERG fusion gene expression in prostate cancer specimens by a novel assay using branched DNA. Urology 74(5):1156-61 (2009).

In some embodiments, Rspo fusion is detected by in situ hybridization. A custom in situ hybridization and signal amplification assay, such as the RNAview® or RNAscope®, is used to detect Rspo fusion transcripts on formalin fixed paraffin embedded (FFPE) or frozen tissues from cancer specimens. The sequences of capture extender probes and the label extender probes are derived from the exon sequences of Rspo genes and fusion partner genes (e.g., PTPRK for Rspo3, EIF3E for Rspo2) such as those exemplified in Example 9. See, Wang F, Flanagan J, Su N, Wang L C, Bui S, Nielson A, Wu X, Vo H T, Ma X J, Luo Y. RNAscope: a novel in situ RNA analysis platform for formalin-fixed, paraffin-embedded tissues. J Mol Diagn. 14(1):22-9 (2012)

In some embodiments, the Rspo fusion is detected by sequencing, such as Sanger sequencing or Next-generation sequencing.

Sequencing by extending a sequencing primer or by extending an extension product can be carried out using a variety of methods. For example, sequencing can be carried out with a labeled reversible terminator or by ligation with a labeled oligonucleotide. Sequencing can be performed using any commercially available method, such as a reversible terminator based sequencing method that is commercially available from companies such as Illumina, Inc. (San Diego, Calif.), and Life Technologies (on Torrent).

In some embodiments, high-throughput sequencing involves the use of technology available from Roche/454 Lifesciences, Inc. (Branford, Conn.). Methods for using bead amplification followed by fiber optics detection are described in Marguiles, M., et al. “Genome sequencing in microfabricated high-density picolitre reactors”, Nature, doi: 10.1038/nature03959; and well as in US Publication Application Nos. 20020012930, 20030058629, 20030100102, 20030148344, 20040248161, 20050079510, 20050124022 and 20060078909.

In some embodiments, high-throughput sequencing is performed using Clonal Single Molecule Array (Solexa, Inc/Illumina, Inc.) or sequencing-by-synthesis (SBS) utilizing reversible terminator chemistry. These technologies are described in part in, e.g., U.S. Pat. Nos. 6,969,488; 6,897,023; 6,833,246; 6,787,308; and US Publication Application Nos. 20040106130, 20030064398, 20030022207, and Constans, A., The Scientist 2003, 17(13):36.

In some embodiments, the method provided herein detects an R-spondin fusion that is (1) a PTPRKe1-Rspo3e2 fusion; (2) a PTPRKe7-Rspo3e2 fusion; (3) an EIF3Ee1-Rspo2e2 fusion; or (4) an EIF3Ee1-Rspo2e3 fusion.

In some embodiments, the method provided herein detects an R-spondin fusion that is (1) an EMC2e1-Rspo2e2 fusion; (2) a PVT1-Rspo2e2 fusion; (3) a PVT1-Rspo2e3 fusion; (4) an HNF4G-Rspo2e2 fusion; or (5) a PTPRKe13-Rspo3e2 fusion.

The R-spondin fusion generally results in expression of R-spondin gene driven by promoter of the fusion partner, such as PTPRK, EIF3E, EMC2, PVT1, or HNF4G gene.

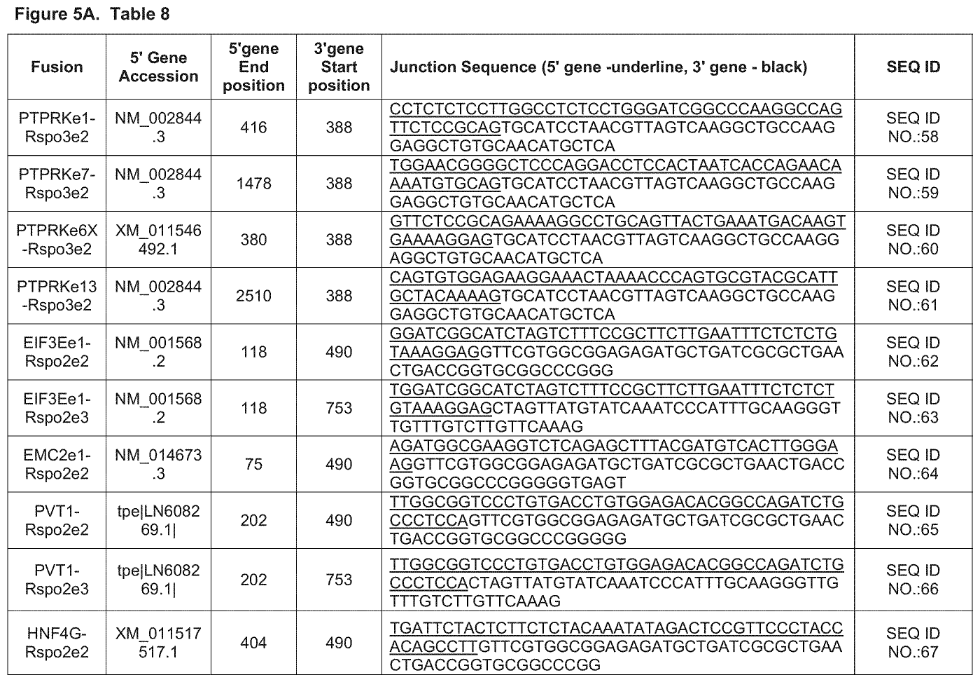

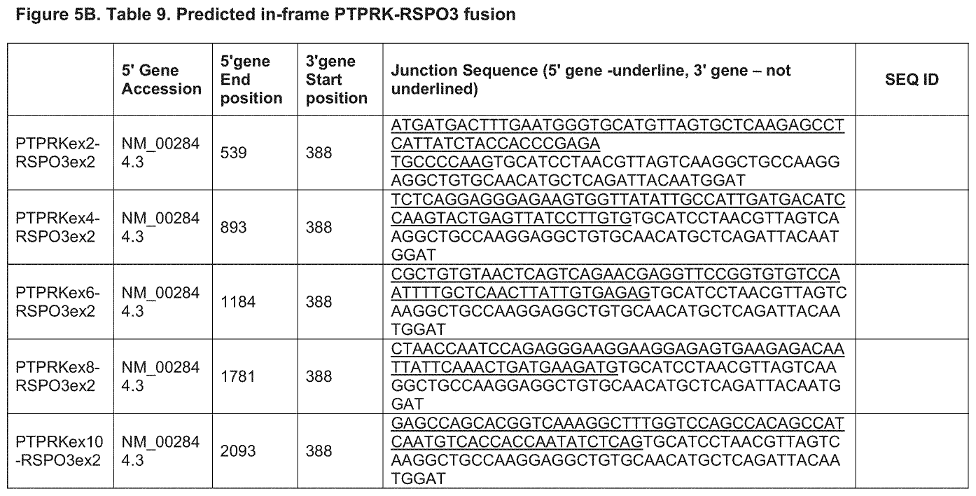

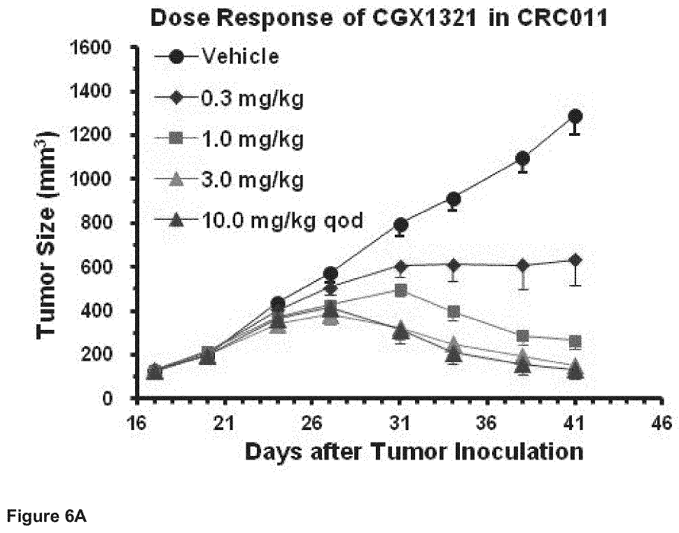

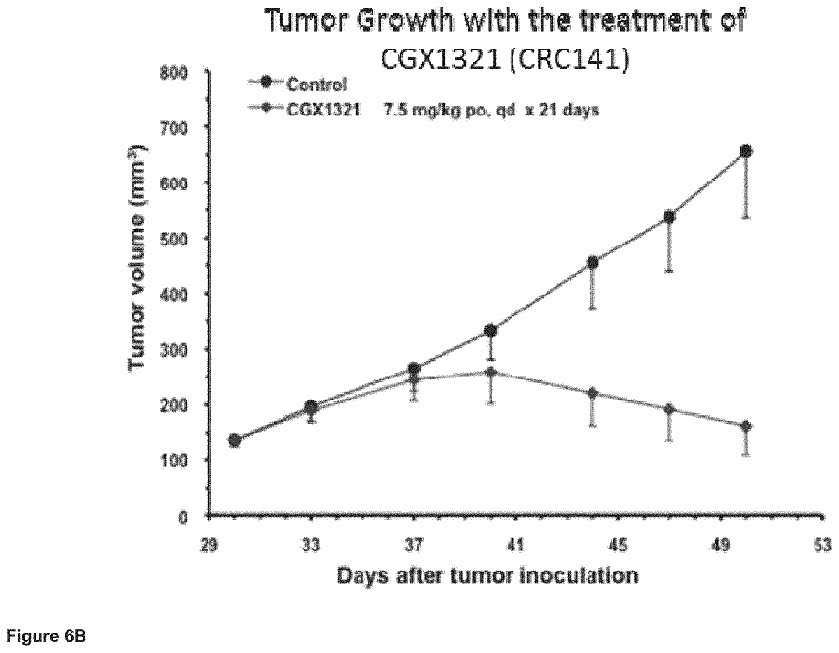

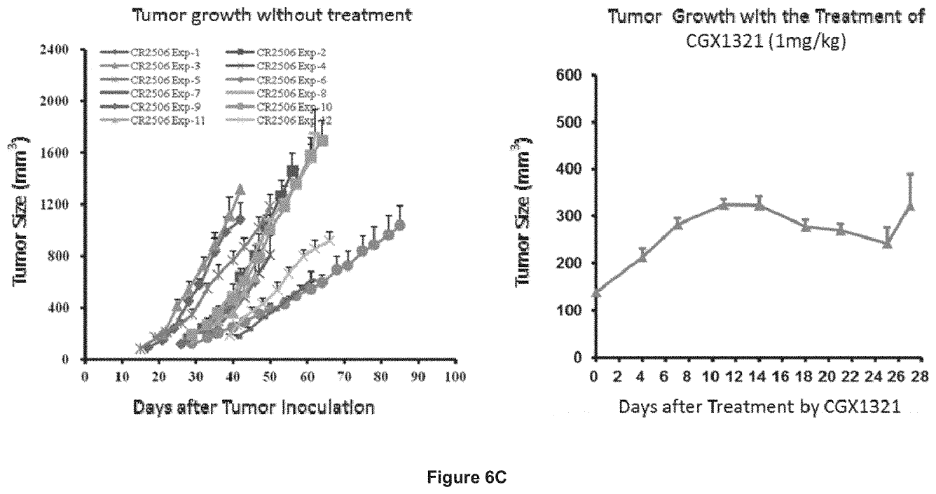

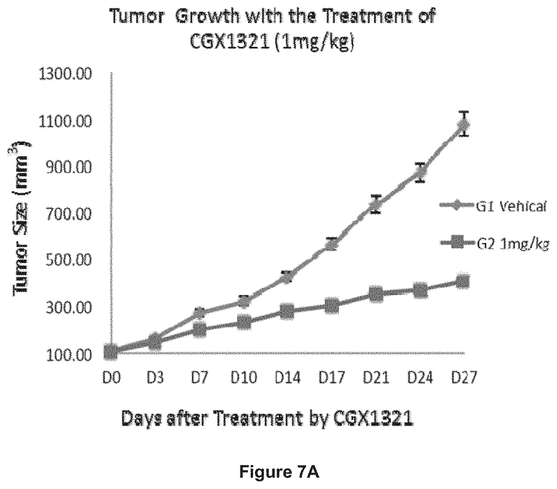

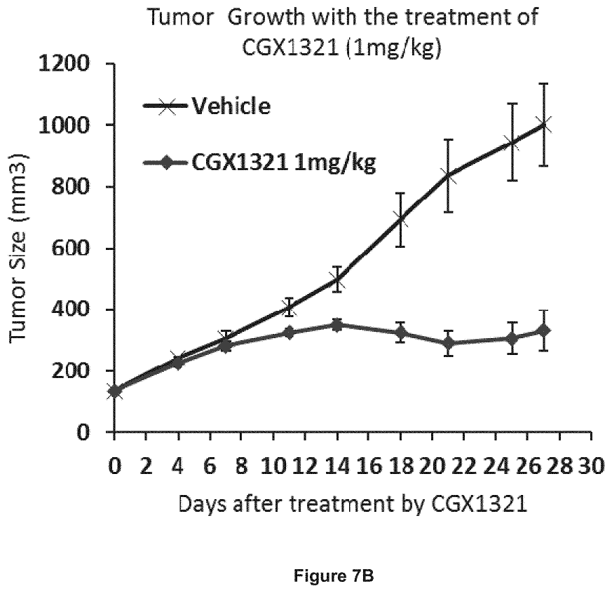

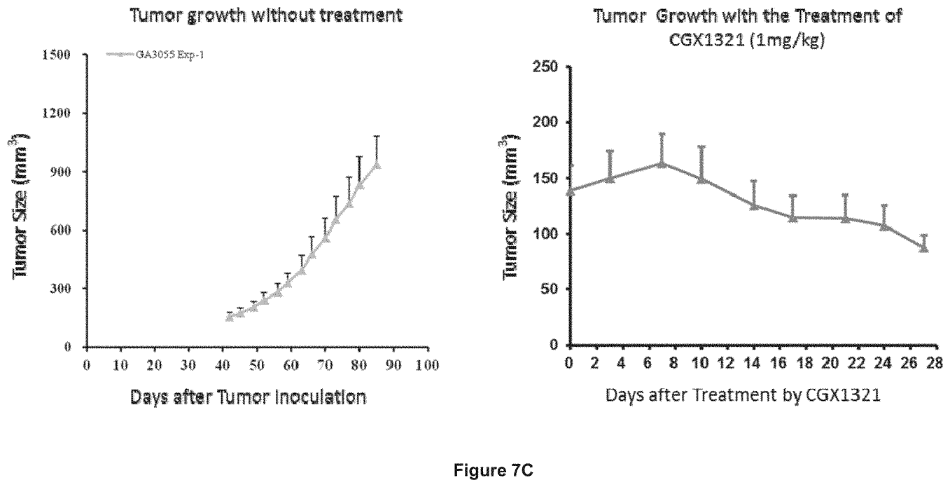

The junction of the various Rspondin gene fusions are provided in Table 8 (FIG. 5A) and Table 9 (FIG. 5B). Also provided are the sequences of the junction of the various gene fusions. It should be apparent to one skilled in the art that that any sequences encompass the junctions as determined by sequencing from a biological sample may include partial or all of the sequences showed in Table 8 (FIG. 5A) and Table 9 (FIG. 5B).

### D. Detection of R-spondin Overexpression

In another aspect, the present invention provides compositions and methods for detection of R-spondin overexpression or elevated expression level. Overexpression of R-spondin may or may not co-exist with overexpression or activation of Wnt.

R-spondin overexpression can be overexpression of either R-spondin mRNA or polypeptide, or both. The R-spondin can be either wild-type or a variant of R-spondin, such as R-spondin fusion as disclosed herein (e.g., Rspo3-PTPRK or Rspo2-EIF3E fusion).

R-spondin overexpression is determined relevant to a baseline expression level, which is obtained by measuring expression level of R-spodin (mRNA or polypeptide) in normal cells or a normal subject population (e.g., normal human population).

The expression level of R-spodin mRNA level is measured using methods known in the art, such as Northern blot, RT-PCR, RT-PCT combined with Real-time PCR, digital PCR, DNA array, high throughput sequencing, or in situ hybridization, Nanostring nCounter, and the like.

The expression level of R-spodin, either at mRNA level or protein level, is measured using methods known in the art, such as Western blot, protein array, immunohistology staining, and the like.

In some embodiments of any of the methods, elevated expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

E. Detection of Rspo Overexpression and/or Rspo Fusion Gene with Nanostring nCounter

In another aspect, R-spondin gene fusion and/or R-spondin overexpression is detected or determined using the nCounter® Analysis system (Nanostring Technologies, Seattle, Wash.). This system is described in International Patent Application Publication No. WO 08/124,847 and U.S. Pat. No. 8,415,102, which are each incorporated herein by reference in their entireties for the teaching of this system.

NanoString does not require amplification of RNA, has low sample requirements and is effective for evaluating the level of gene expression in FFPE samples, such as tumor FFPE samples. Furthermore, NanoString is a multiplexed method for detecting gene expression and provides a method for direct measurement of mRNAs without the use of transcription or amplification. The RNA extracted from formalin fixed tumor specimens may be of very poor quality and until recently no such analysis was possible. NanoString, however, allows for analysis of these specimens. With a sensitivity of 500 attomolar NanoString can detect as little as one copy of RNA per cell using 100 nanograms of total RNA as input.

The basis of the nCounter® Analysis system is the unique code assigned to each nucleic acid target to be assayed. The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system.

Specific reporter and capture probes are synthesized for each target. Briefly, sequence-specific DNA oligonucleotide probes are attached to code-specific reporter molecules. Preferably, each sequence specific reporter probe comprises a target specific sequence capable of hybridizing to no more than one gene of interest (e.g. Rspo2, Rspo3, or one of their fusion gene counterpart) and optionally comprises at least two, at least three, or at least four label attachment regions, and the attachment regions comprising one or more label monomers that emit light. Capture probes are made by ligating a second sequence-specific DNA oligonucleotide for each target to a universal oligonucleotide containing biotin. Reporter and capture probes are all pooled into a single hybridization mixture, the “probe library”. Preferably, the probe library comprises a probe pair (a capture probe and reporter) for each of the genes of interest as provided herein.

The relative abundance of each target is measured in a single multiplexed hybridization reaction. The method comprises contacting a biological sample with a probe library, the library comprising a probe pair for the genes of interest, such that the presence of the target in the sample creates a probe pairs and target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled roboticaly on a custom liquid-handling robot (Prep Station, NanoString Technologies).

Purified reactions are deposited by the Prep Station into individual flow cells of a sample cartridge, bound to a streptavidm-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. Data is output in simple spreadsheet format listing the number of counts per target, per sample.

This system can be used along with nanoreporters. Additional disclosure regarding nanoreporters can be found in International Publication No. WO 07/076,129 and WO 07/076,132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

One advantage of the nCounter system is the capacity to measure both gene fusion and gene overexpression in a single assay. nCounter Element Chemistry assay enable multiplexed assays capable of detecting and discriminating over 200 expressed gene and gene fusions in a single reaction. Known gene fusions can be characterized with specific probe pairs targeting fusion junction sequence. Novel fusion genotypes without knowledge of partner genes can be identified by the 5′ and 3′ exon imbalance. The ratio of exons expression 5′ upstream and 3′ downstream of the fusion junction can be robustly assessed with sequence specific probes. A ratio of 5′/3′ expression that diverges from 1 is therefore indicative that a fusion event has occurred. Increased Rspo2 and Rspo3 expression driven by their 5′ fusion partner genes in cancer stem cells is additional strong indicator to a fusion event. See Lira M E, Kim T M, Huang D, Deng S, Koh Y, Jang B, Go H, Lee S H, Chung D H, Kim W H, Schoenmakers E F, Choi Y L, Park K, Ahn J S, Sun J M, Ahn M J, Kim D W, Mao M. Multiplexed gene expression and fusion transcript analysis to detect ALK fusions in lung cancer. J Mol Diagn 2013 15(1):51-61. Lira M E, Choi Y L, Lim S M, Deng S, Huang D, Ozeck M, Han J, Jeong J Y, Shim H S, Cho B C, Kim J, Ahn M J, Mao M. A single-tube multiplexed assay for detecting ALK, ROS1, and RET fusions in lung cancer. J Mol Diagn 2014 16(2):229-243.

NanoString and aspects thereof are described in Geiss et al., “Direct multiplexed measurement of gene expression with color-coded probe pairs” Nature Biotechnology 26, 317-325 (2008); in U.S. Pat. Nos. 7,473,767, 7,941,279 and 7,919,237, and in U.S. Patent Application Publication No. 2010/0112710, the entire contents of each of which are hereby incorporated by reference. NanoString is also discussed in: Payton et al., “High throughput digital quantification of mRNA abundance in primary human acute myeloid leukemia samples” The Journal of Clinical Investigation 119(6):1714-1726 (2009); and Vladislav et al. “Multiplexed measurements of gene signatures in different analytes using the NanoStringnCounter Assay System” BMC Research Notes 2: 80 (2009), the entire contents of each of which are hereby incorporated by reference.

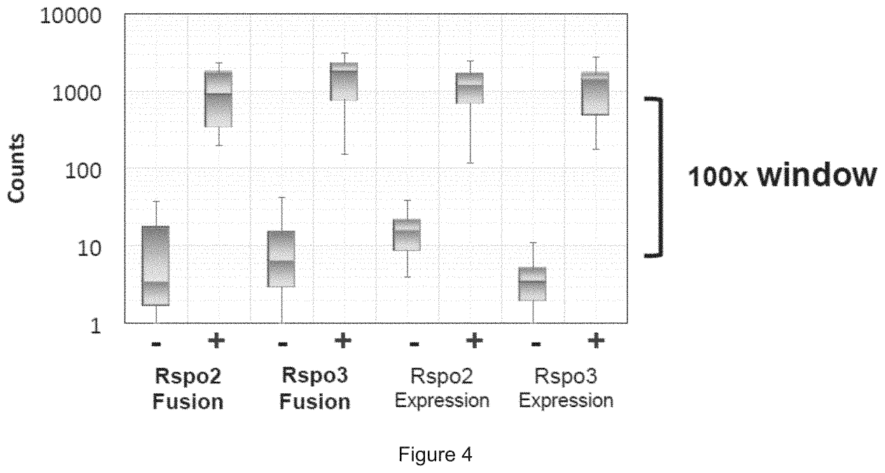

Gene expression of R-Spondin is in general correlated with WNT pathway activation, and minimal in most differentiated tissues with inactivated WNT signaling. When Rspo2 and Rspo3 coding genes are fused to the 3′ end of an actively transcribed gene, their expression is significantly elevated and potentially drives tumorgenesis. FIG. 4 depicts the Nanostring nCounter quantification of Rspo2 and Rspo3 transcripts in 100 ng total RNA of tumors. Rspo2 or Rspo3 transcripts in the tumors harboring Rspo2 or Rspo3 fusion gene are more than 100× compared to that in the tumors without a fusion. In addition, 5′-end exons are absent when Rspo2 and Rspo3 fused to their partners, resulting in the imbalance of 5′ and 3′ exons. Thus, the amount of 5′-end exons is notable lower than the amount of 3′ exons in Rspo2 and Rspo3 mRNAs, which they fuse to their partner genes.

In some embodiments, the overexpression of Rspo2 and/or Rspo3 fusion is more than 1, 2, 3, 4, 5, 6, 7, 8, 9, 10, 20, 30, 40, 50, 60, 70, 80, 90, 100, or more fold thatn in the tumor (or normal tissue) without the fusion.

F. Screening and Treatment of Subject with Rspo Overexpression and/or Rspo Fusion Gene

In another aspect, the present invention provides a method for determining whether a subject with cancer should be treated with a composition that inhibits Wnt activity, such as a Procupine antagonist or inhibitor the method comprising: (a) isolating a biological sample from the subject; (b) performing an assay on the biological sample to determine expression of Rspo mRNA or polypeptide and/or identify the presence or absence of an R-spondin fusion; and (c) determining that the subject should be treated with a composition that inhibits Porcupine activity if the biological sample contains Rspo mRNA or polypeptide overexpression and/or an R-spondin fusion, wherein the composition comprises a Porcupine inhibitor provided herein.

In some embodiments, the method further comprises treating the subject with the composition provided herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### IV. Assays and Kits for Screening of Subject with Rspo Overexpression and/or Rspo Fusion Gone

In another aspect, the present invention provides kus for screening of a subject (e.g., a human patient) for Rspo2 and/or Rspo3 gene fusions and/or over expression.

The assay kits and methods of the invention may be used to identify patient, cell, or tissue that is predicted to be responsive to a particular Wnt inhibitor. The use of such a companion diagnostic kit would be similar to other companion diagnostic tests approved by governmental drug registration agencies for use with approved drugs. See, for example, the approvals by the Food and Drug Administration in 2011 of crizotinib for the treatment of ALK4-mutated lung cancer and of vemurafenib for BRAF-mutated melanoma.

The assay kits and methods of the invention may also be useful for identifying treatments that can improve the responsiveness of cancer cells which are resistant to Wnt inhibitors, and to develop adjuvant treatments that enhance the response of the Wnt inhibitors.

The assay kits and methods of the invention are useful to patients with any cancer that can be treated with Wnt inhibitors, such as or pancreatic cancer or colon cancer, or any tumors whose growth can be slowed by Wnt inhibitors, such as ductal carcinomas, adenocarcinomas or melanomas. Such patients may, as a result of the methods provided herein, be spared from side effects and financial costs of an ineffective therapy in the event that they do not have Rspo2 or Rspo2 gene fusions and/or overexpression. The assay kits and methods of the invention are also useful to physicians, who can recommend, a Wnt inhibitor therapy, or not, to particular patients based on information on the molecular characteristics of their tumors. The assay kits and methods of the invention will also usefully increase the demand for development of an efficient human Rspodin assay to be made available with yet-to-be developed nucleotide probes.

In one embodiment, the invention provides an assay kit for selecting a cancer patient who is predicted to benefit or not to benefit from therapeutic administration of a Wnt inhibitor. The assay kit includes:

(a) a means or system for detecting in a sample of tumor cells a level of a biomarker or a combination of biomarkers selected from: (i) a Rspo 2 and/or Rspo 3 gene fusion; or (ii) a level of expression of Rspo2 and/or Rspo 3 genes.
(b) a control selected from: (i) a control sample for detecting sensitivity to the Wnt inhibitor; (ii) a control sample for detecting resistance to the Wnt inhibitor; (iii) information containing a predetermined control level of the biomarker that has been correlated with sensitivity to the Wnt inhibitor; or (iv) information containing a predetermined control level of the biomarker that has been correlated with resistance to the Wnt inhibitor.

In one embodiment, the kit can further include a means system for detecting a fusion of the Rspo2 gene or Rspo3 gene.

In one embodiment, the means for detecting the mutation is a nucleotide probe that hybridizes to a portion of the Rspo2 gene or Rspo33 gene. In a particular embodiment, the means for detecting is a fluorescent in situ hybridization (FISH) probe. Any of the means for detecting can contain a detectable label. Any of the means for detecting can be immobilized on a substrate.

The assay kit may also include one or more controls. The controls could include: (i) a control sample for detecting sensitivity to the Wnt inhibitor being evaluated for use in a patient; (ii) a control sample for detecting resistance to the Wnt inhibitor; (iii) information containing a predetermined control level of particular biomarker to be measured with regard to Wnt inhibitor sensitivity or resistance (e.g., a predetermined control level of Rspo2 and/or Rspo3 gene fusion and/or overexpression level that has been correlated with sensitivity to the Wnt inhibitor or resistance to Wnt inhibitor).

The kit can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a biomarker described previously herein. Specifically, the means is characterized in that it identifies a specific marker of the cell type being analyzed that positively identifies the cell type. For example, in a lung tumor assay, it is desirable to screen lung epithelial cells for the level of the biomarker expression or biological activity. Therefore, the means for detecting a control marker identifies a marker that is characteristic of an epithelial cell and preferably, a lung epithelial cell, so that the cell is distinguished from other cell types, such as a connective tissue or inflammatory cell. Such a means increases the accuracy and specificity of the assay of the invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally distinct site on the target molecule; or an antibody, antigen binding fragment thereof, or antigen binding peptide that selectively binds to the control marker in the sample. Nucleic acid and amino acid sequences for many cell markers are known in the art and can be used to produce such reagents for detection.

In some embodiments, the assay or kit include the probes and other necessary reagents of the nCounter system. Nanostring nCounter assay can be conducted in multiple designs in detecting fusion junctions and assessing gene expression: (1) Codeset design employs two ˜50 base probes per mRNA that hybridize in solution. The Reporter Probe carries the signal; the Capture Probe allows the complex to be immobilized for data collection; (2) Element Tagset GRP design utilizes digital, molecular barcoding chemistry based on NanoString's patented technology that allows users to assemble their own assays; and 3) universal junction sequence design utilizes toehold exchange technology to enable highly specific detection.

### REFERENCE

Akiri G, Cherian M M, Vijayakumar S, Liu G, Bafico A, Aaronson S A. Wnt pathway aberrations including autocrine Wnt activation occur at high frequency in human non-small-cell lung carcinoma. Oncogene. 2009 May 28; 28(21):2163-72.
Bafico A, Liu G, Goldin L, Harris V, Aaronson S A. An autocrine mechanism for constitutive Wnt pathway activation in human cancer cells. Cancer Cell. 2004 November; 6(5):497-506.
Barker N, Clevers H. Mining the Wnt pathway for cancer therapeutics. Nat Rev Drug Discov. 2006 December; 5(12):997-1014.
Blom A B, van Lent P L, van der Kraan P M, van den Berg W B. To seek shelter from the WNT in osteoarthritis? WNT-signaling as a target for osteoarthritis therapy. Curr Drug Targets. 2010 May; 11(5):620-9.
Boonen R A, van Tijn P, Zivkovic D. Wnt signaling in Alzheimer's disease: up or down, that is the question. Ageing Res Rev. 2009 April; 8(2):71-82.
Camilli T C, Weeraratna A T. Striking the target in Wnt-y conditions: intervening in Wnt signaling during cancer progression. Biochem Pharmacol. 2010 Sep. 1; 80(5):702-11.
Chan S L, Cui Y, van Hasselt A, Li H, Srivastava G, Jin H, Ng K M, Wang Y, Lee K Y, Tsao G S, Zhong S, Robertson K D, Rha S Y, Chan A T, Tao Q. The tumor suppressor Wnt inhibitory factor 1 is frequently methylated in nasopharyngeal and esophageal carcinomas. Lab Invest. 2007 July; 87(7):644-50.
Chen B, Dodge M E, Tang W, Lu J, Ma Z, Fan C W, Wei S, Hao W, Kilgore J, Williams N S, Roth M G, Amatruda J F, Chen C, Lum L. Small molecule-mediated disruption of Wnt-dependent signaling in tissue regeneration and cancer. Nat Chem Biol. 2009 February; 5(2):100-7.
Cheng J H, She H, Han Y P, Wang J, Xiong S, Asahina K, Tsukamoto H. Wnt antagonism inhibits hepatic stellate cell activation and liver fibrosis. Am J Physiol Gastrointest Liver Physiol. 2008; 294(1):G39-49.
Chun J S, Oh H, Yang S, Park M. Wnt signaling in cartilage development and degeneration. BMB Rep. 2008 Jul. 31; 41(7):485-94.
Chien A J, Moon R T. WNTS and WNT receptors as therapeutic tools and targets in human disease processes. Front Biosci. 2007 Jan. 1; 12:448-57.
DeAlmeida V I, Miao L, Ernst J A, Koeppen H, Polakis P, Rubinfeld B. The soluble wnt receptor Frizzled-8CRD-hFc inhibits the growth of teratocarcinomas in vivo. Cancer Res. 2007 Jun. 1; 67(11):5371-9
D'Amour K A, Bang A G, Eliazer S, Kelly O G, Agulnick A D, Smart N G, Moorman M A, Kroon E, Carpenter M K, Baetge E E. Production of pancreatic hormone-expressing endocrine cells from human embryonic stem cells. Nat Biotechnol. 2006 November; 24(11):1392-401. Herbst A, Kolligs F T. Wnt signaling as a therapeutic target for cancer. Method Mol Biol. 2007; 361:63-91.
Hoeppner L H, Secreto F J, Westendorf J J. Wnt signaling as a therapeutic target for bone diseases. Expert Opin Ther Targets. 2009 Apr. 13 (4):485-96.
Hwang I, Seo E Y, Ha H. Wnt/beta-catenin signaling: a novel target for therapeutic intervention of fibrotic kidney disease. Arch Pharm Res. 2009 December; 32(12):1653-62.
Inestrosa N C, Arenas E. Emerging roles of Wnts in the adult nervous system. Nat Rev Neurosci. 2010 February; 11(2):77-86.
Kansara M, et al. Wnt inhibitory factor 1 is epigenetically silenced in human osteosarcoma, and targeted disruption accelerates osteosarcomagenesis in mice. J Clin Invest. 2009 April; 119(4):837-51
Lie D C, Colamarino S A, Song H J, Désiré L, Mira H, Consiglio A, Lein E S, Jessberger S, Lansford H, Dearie A R, Gage F H. WNT signalling regulates adult hippocampal neurogenesis. Nature 437 (7063):1370-5, 2005.
Lira M E, Kim T M, Huang D, Deng S, Koh Y, Jang B, Go H, Lee S H, Chung D H, Kim W H, Schoenmakers E F, Choi Y L, Park K, Ahn J S, Sun J M, Ahn M J, Kim D W, Mao M. Multiplexed gene expression and fusion transcript analysis to detect ALK fusions in lung cancer. J Mol Diagn 2013 15(1):51-61.
Lira M E, Choi Y L, Lim S M, Deng S, Huang D, Ozeck M, Han J, Jeong J Y, Shim H S, Cho B C, Kim J, Ahn M J, Mao M. A single-tube multiplexed assay for detecting ALK, ROS1, and RET fusions in lung cancer. J Mol Diagn 2014 16(2):229-243.
MacDonald B T, Tamai K, He X. Wnt/beta-catenin signaling: components, mechanisms, and diseases. Dev Cell. 2009 July; 17(1):9-26.
Mikels A J, Nusse R. Wnts as ligands: processing, secretion and reception. Oncogene. 2006 Dec. 4; 25(57):7461-8.
Moon R T. Wnt/beta-catenin pathway. Sci STKE.; 2005(271):cm1.
Morrisey E E. Wnt signaling and pulmonary fibrosis. Am J Pathol. 2003 May; 162(5):1393-7.
Nusse R. WNT signaling and stem cell control”. Cell Res. 18 (5): 523-7, 2008.
Ouchi N, Higuchi A, Ohashi K, Oshima Y, Gokce N, Shibata R, Akasaki Y, Shimono A, Walsh K. Sfrp5 is an anti-inflammatory adipokine that modulates metabolic dysfunction in obesity. Science. 2010 Jul. 23; 329(5990):454-7.
Reya T, Clevers H. Wnt signalling in stem cells and cancer. Nature. 2005 Apr. 14; 434(7035):843-50.
Rhee C S, Sen M, Lu D, Wu C, Leoni L, Rubin J, Corr M, Carson D A. Wnt and frizzled receptors as potential targets for immunotherapy in head and neck squamous cell carcinomas. Oncogene. 2002 Sep. 26; 21(43):6598-605.
Sullivan G J, et al. Generation of functional human hepatic endoderm from human induced pluripotent stem cells. Hepatology. 2010 January; 51(1):329-35.
Takahashi-Yanaga F, Kahn M. Targeting Wnt signaling: can we safely eradicate cancer stem cells? Clin Cancer Res. 2010 Jun. 15; 16(12):3153-62.
Ten Berge, D. et al. WNT signaling mediates self-organization and axis formation in embryoid bodies. Cell Stem Cell 3, 508-518, 2008.
Yang L, Soonpaa M H, Adler E D, Roepke T K, Kattman S J, Kennedy M, Henckaerts E, Bonham K, Abbott G W, Linden R M, Field L J, Keller G M. Human cardiovascular progenitor cells develop from a KDR+ embryonic-stem-cell-derived population. Nature. 2008 May 22; 453(7194):524-8.

### EXAMPLES

The present invention is further exemplified, but not limited, by the following and Examples that illustrate the preparation of the compounds of the invention.

| Abbreviation | Definition or Explanation |
|---|---|
| DCM | Dichloromethane |
| DIEA | N,N’-Diisopropylethylamine |
| DMF | N,N-Dimethylformamide |
| eq. | equivalents |
| TEA | Triethylamine |
| THF | Tetrahydrofuran |
| RT | Room Temperature |
| EA | Ethyl acetate |
| Pd₂(dba)₃ | Tris(dibenzylideneacetone)dipalladium(0) |
| s-Phos | 2-Dicyclohexylphosphino-2′,6′-dimethoxybiphenyl |
| Pd(PPh₃)₄ | Tetrakis(triphenylphosphine)palladium |

### Example 1

### Synthesis of N-(4-(2-methylpyridin-4-yl)benzyl-6-(2-methylpyridin-4-vi-2,7-naphthyridin-1-amine (Compound No. 1)

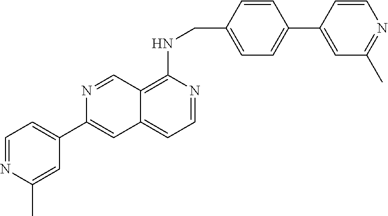 [SMILES: Cc1cc(-c2ccc(CNc3nccc4cc(-c5ccnc(C)c5)ncc34)cc2)ccn1]

Step 1:

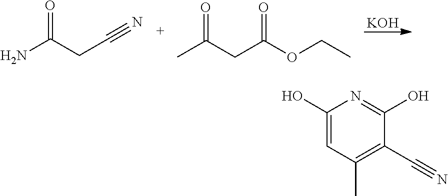

2-Cyanoacetamide (50 g, 601.8 mmol) and ethyl acetoacetate (75 mL, 601.8 mmol) were dissolved in MeOH. KOH (37.0 g, 1.1 eq) was dissolved in MeOH, and added dropwise into the mixture, some white solid came out. The mixture was heated up to reflex at oil bath for 8 h, and then cooled down to RT. The solid was filtered and then re-dissolved into hot water, and then filtered again. 6N HCl was added into the filtration to neutralize till pH<7. The white solid was out again and filtered. The solid was further washed with MeOH, water and MeOH, and then dried by vacuum to get the final product 3-ethynyl-4-methylpyridine-2,6-diol (yield ˜41%).

Step 2:

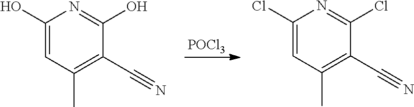

3-ethynyl-4-methylpyridine-2,6-diol (28.0 g, 195.2 mmol) was dissolved in POCl₆ (60.0 mL). The reaction mixture was sealed in a pressure tube and heated up to 180° C. for 6 h. After the reaction was cooled down to room temperature, the excessive POCl₃ was removed under the vacuum. Slowly added crushed ice into the mixture, and the solid came out. Filtered the solid out and dried under the vacuum to get the final product 2,6-dichloro-4-methylpyridine-3-carbonitrile (yield ˜92%) without further purity.

Step 3:

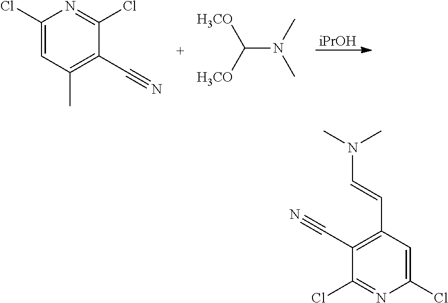

2,6-dichloro-4-methylpyridine-3-carbonitrile (20.0 g, 107.5 mmol) in 200 mL of isopropyl alcohol was added N,N-dimethylformamide dimethylacetal (12.82 g, 107.5 mmol) and the reaction was stirred at 65° C. for 18 h. After cooling down the reaction to RT, the precipitate was collected by filtration and washed with 50 mL of isopropyl alcohol, and air dried to give the product 2,6-dichloro-4-((E)-2-(dimethylamino)vinyl)pyridine-3-carbonitrile (yield˜26%) without further purification.

Step 4:

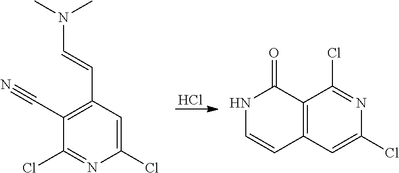

2,6-dichloro-4-((E)-2-(dimethylamino)vinylpyridine-3-carbonitrile (4.0 g, 16.6 mmol) was added with 20 mL concentrated HCl in a sealed tube. The reaction is stirred at 45° C. for 18 h. After cooling down the reaction to RT, ice water was added to the solution resulting heavy yellow slurry. The precipitate was collected by filtration, washed with cold water, ether and ethyl acetate, and dried under vacuum to get light yellow solid 6,8-dichloro-2,7-naphthyridin-1(2H)-one (yield ˜80%). MS m/z 215.0 (M+1). ¹HNMR (300 MHz, DMSO-d6): δ11.75 (s, 1H), 7.76 (s, 1H), 7.50 (t, J=6.6 Hz, 1H), 6.52 (d, J=6.6 Hz, 1H).

Step 5:

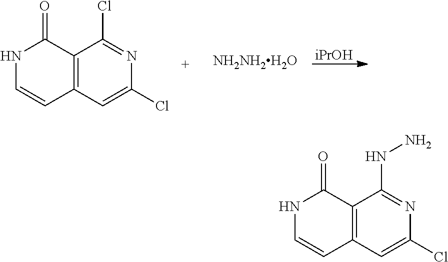 [SMILES: "iPrOH" ; O=c1[nH]ccc2cc(Cl)nc(Cl)c12 ; "NH2NH2·H2O" ; NNc1nc(Cl)cc2cc[nH]c(=O)c12]

6,8-dichloro-2,7-naphthyridin-1(2H)-one (3.0 g, 13.96 mmol) was dissolved in iPrOH (120 mL) to form a kind of suspension. The solution was cooled down to 0° C. in ice bath, and then hydrazine solution (5.6 g, 80%, 10 eq) was added dropwise. The mixture was stirred at RT for 15 minutes, and then heated in oil bath at 55° C. for overnight. After the reaction mixture was cooled down to RT, filtered to get the solid directly, and then the solid was washed with 70 mL MeOH and dried by vacuum. The product 6-chloro-8-hydrazinyl-2,7-naphthyridin-1(2H)-one (yield ˜98%) was used in the next step reaction directly without further purification.

Step 6:

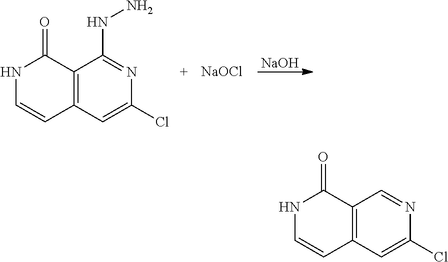 [SMILES: NNc1nc(Cl)cc2cc[nH]c(=O)c12 ; "NaOCl" ; "NaOH" ; O=c1[nH]ccc2cc(Cl)ncc12]

6-chloro-8-hydrazinyl-2,7-naphthyridin-1(2H)-one (1.50 g, 7.12 mmol) was dissolved into MeCN (90 mL) to form a kind of suspension. 1N NaOH (17.80 mL, 2.5 eq) was added, and then equal amount of water (107.80 mL) was added into the mixture. The reaction mixture was heated at 50° C., stirred till becoming the clear solution. The solution was cooled down to 0° C. again, and NaOCl (11.05 g, 12% solution, 2.5 eq) was added dropwise, and then reaction was stirred at RT for overnight. After the reaction was done, the solution was cooled down to 0° C. and then added into 1N HCl to neutralize (pH ˜6). Precipitate was collected and the filtrate was extracted with 100 mL×2 EA. The organic layer was combined and dried over Na₂SO₄ and evaporated to give additional crude product. The combined solid material 6-chloro-2,7-naphthyridin-1(2H)-one (yield ˜93%) was used in the next reaction without further purification. MS m/z 181.1 (M+1).

Step 7:

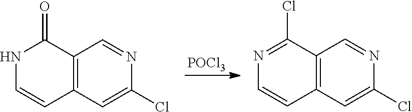

6-chloro-2,7-naphthyridin-1(2H)-one (400 mg, 2.2 mmol) was added in POCl₃ (20.0 mL) in a pressure tube. The reaction mixture was heated up to 160° C. for 4 h to get a clear solution. The solution was cooled down to room temperature and poured in DCM, and added crushed ice slowly. Saturated NaHCO₃ was added into the mixture to neutralize HCl generated in the reaction. Vacuum to remove DCM and the left water solution was extracted by 100 mL×2 EA. The combined organic layers were washed with brine once, and dried by Na₂SO₄, and then evaporated under the vacuum to get the solid 1,6-dichloro-2,7-naphthyridine (yield ˜73%) to use in the next step reaction without further purifications. MS m/z 199.0 (M+1).

Step 8:

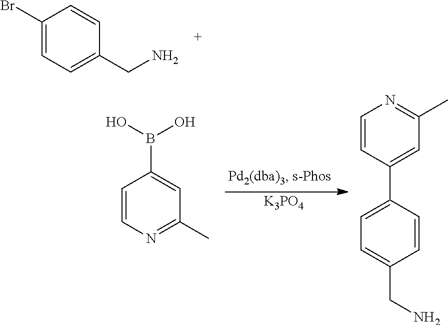

(4-bromophenyl)methanamine (1.00 g, 5.37 mmol) and 2-methylpyridin-4-yl-4-boronic acid (883.30 mg, 6.45 mmol) were dissolved in BuOH (10.0 mL) and water (2.0 mL). K₃PO₄ (2.28 g, 10.75 mmol), Pd₂(dba)₃ (120.20 mg, 0.27 mmol) and S-phos (220.70 mg, 0.54 mmol) were added in under N₂. The reaction mixture was sealed in a pressure tube and heated up to 125° C. for 1 h. After cooling down the reaction to RT, the mixture was poured into the water and extracted by 100 mL×3 EA. The combined organic layer was washed with brine, dried over Na₂SO₄, and concentrated under the vacuum to give the crude product. The solid was purified by silicone gel column with 10% MeOH (containing ˜2N NH₃) in DCM to get the pure (4-(2-methylpyridin-4-yl)phenyl)methanamine (yield˜89%). MS m/z 199.1 (M+1).

Step 9:

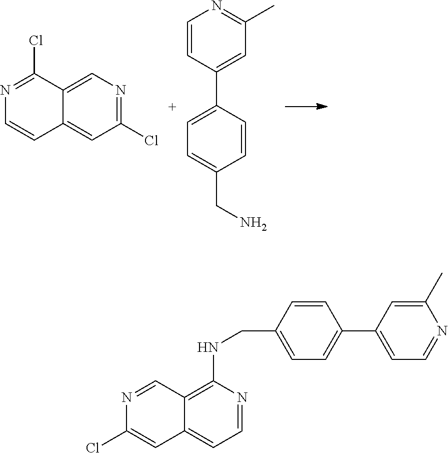

1,6-dichloro-2,7-naphthyridine (160 mg, 0.80 mmol) and (4-(2-methylpyridin-4-yl)phenyl)methanamine (239.10 mg, 1.21 mmol) were dissolved in BuOH (5.0 mL) and heated up to 115° C. for overnight. After the reaction was cooled down to RT, the organic solvent was removed under the vacuum. The crude product was purified by silicone gel flash chromatography with EA/hexane (1:1) to get the solid N-(4-(2-methylpyridin-4-yl)benzyl)-8-chloro-2,7-naphthyridin-1-amine (yield ˜90%). MS m/z 361.1 (M+1).

Step 10:

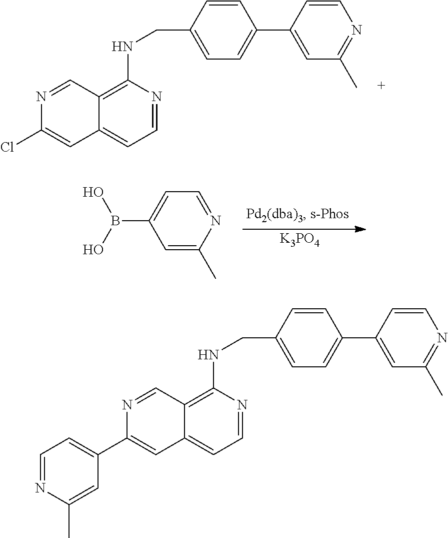

N-(4-(2-methylpyridin-4-yl)benzyl)-6-chloro-2,7-naphthyridin-1-amine (50.00 mg, 0.14 mmol) and 2-methylpyridin-4-yl-4-boronic acid (56.90 mg, 0.42 mmol) were dissolved in BuOH (3.0 mL) and water (0.6 mL). K₃PO₄ (88.20 mg, 0.028 mmol), Pd₂(dba)₃ (6.20 mg, 0.014 mmol) and S-phos (11.40 mg, 0.011 mmol) were added into the mixture under N₂. The reaction was sealed in a pressure tube and heated up to 105° C. for overnight. After cooling down the reaction to RT, the mixture was poured in water and extracted by EA for three times. The combined organic layer was washed with brine, dried by Na₂SO₄, and concentrated under the vacuum. The crude product was further purified by prep-TLC with 5% MeOH in DCM to get the final product N-(4-(2-methylpyridin-4-yl)benzyl)-6-(2-methylpyridin-4-yl)-2,7-naphthyridin-1-amine (yield ˜70%). MS m/z 418.2 (M+1). ¹HNMR (300 MHz, CDCl₂): δ2.46 (s, 3H), 2.63 (s, 3H), 4.94 (d, J=5.10 Hz, 2H), 5.94 (br, 1H), 6.97 (d, J=5.70 Hz, 1H), 7.31 (d, J=4.20 Hz, 1H), 7.36 (s, 1H), 7.54 (d, J=8.10 Hz, 2H), 7.63 (d, J=8.40 Hz, 2H), 7.90 (s, 1H), 8.19 (d, J=6.00 Hz, 1H), 8.22 (s, 1H), 8.51 (m, 2H), 9.08 (s, 1H), 9.30 (s, 1H).

### Example 2

### Synthesis of N-(3-methyl-4-(2-methylpyridin-4-yl)benzyl)-6-(2-methylpyridin-4-yl)-2,7-naphthyridin-1-amine (Compound No. 2)

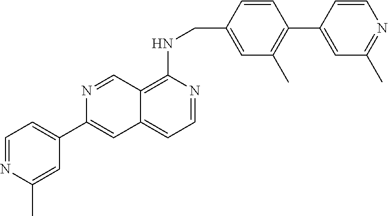 [SMILES: Cc1cc(-c2cc3ccnc(NCc4ccc(-c5ccnc(C)c5)c(C)c4)c3cn2)ccn1]

Step 1:

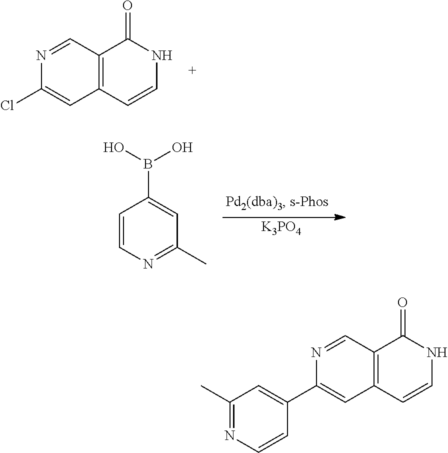

6-chloro-2,7-naphthyridin-1(2H)-one (200 mg, 1.10 mmol) and 2-methylpyridin-4-yl-4-boronic acid (227.60 mg, 1.66 mmol) were dissolved in BuOH (5.0 mL) and water (1.0 mL). K₃PO₄ (705.20 g, 3.32 mmol), Pd₂(dba)₃ (49.60 mg, 0.22 mmol) and S-phos (91.00 mg, 0.11 mmol) were added under N₂. The reaction mixture in the pressure tube was heated up to 130° C. for 1 h. After cooling down the reaction to RT, poured the mixture into the water, extracted by EA for three times. The combined organic layer was washed with brine, dried over Na₂SO₄, concentrated under the vacuum to get the crude. The crude product was purified by column with 5% MeOH in DCM to get the final compound 6-(2-methylpyridin-4-yl)-2,7-naphthyridin-1(2H)-one (yield˜61%). MS m/z 238.1 (M+1).

Step 2:

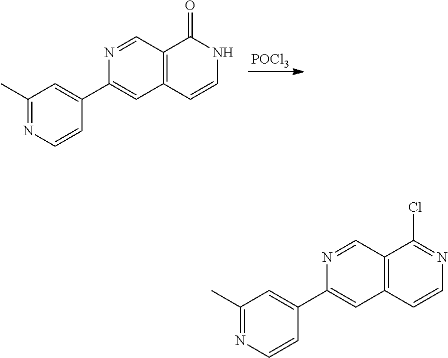

6-(2-methylpyridin-4-yl)-2,7-naphthyridin-1(2H)-one (150 mg, 0.63 mmol) was dissolved in POCl₃(15.0 mL), the pressure tube was sealed and heated up to 160° C. for 4 h. After cooling down the reaction to RT, excessive POCl₃ was removed under vacuum. Crushed ice was slowly added into the mixture, and then added into NaHCO₃ to neutralize until pH ˜7.5. Extracted the solution by EA three times, the combined organic layer was washed with brine, dried over Na₂SO₄, and concentrated under vacuum. The crude was purified by column with EA/hexane (1:1) to get the compound 1-chloro-6-(2-methylpyridin-4-yl)-2,7-naphthyridine (yield ˜55%). MS m/z 256.1 (M+1).

Step 3:

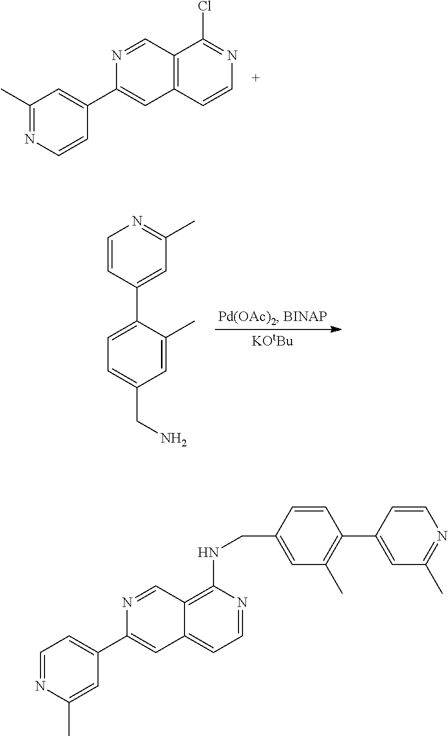

1-chloro-6-(2-methylpyridin-4-yl)-2,7-naphthyridine (10.00 mg, 0.039 mmol) and (3-methyl-4-(2-methylpyridin-4-yl))phenyl)methanamine (10.00 mg, 0.047 mmol) were dissolved in toluene (1.0 mL). KOtBu (8.80 mg, 0.078 mmol), Pd(OAc)₂ (0.90 mg, 0.0039 mmol) and BINAP (4.90 mg, 0.0078 mmol) was added into the mixture under N₂. The reaction was heated up to 100° C. overnight. After cooling down the reaction to RT, poured the mixture into the water, extracted by EA for three times. The combined organic layer was washed with brine, dried over Na₂SO₄, then concentrated under vacuum. The crude product was purified by prep-TLC by EA/hexane (4:1) to get N-(3-methyl-4-(2-methylpyridin-4-yl)benzyl)-6-(2-methylpyridin-4-yl)-2,7-naphthyridin-1-amine (8.8 mg, yield ˜52%). 1H NMR (300 MHz, CDCl₃): δ2.31 (s, 3H), 2.63 (s, 3H), 2.70 (s, 3H), 4.91 (d, J=5.10 Hz, 2H), 5.88 (br, 1H), 7.00 (d, J=5.40 Hz, 1H), 7.08 (d, J=5.10 Hz, 1H), 7.12 (s, 1H), 7.22 (d, J=7.50 Hz, 1H), 7.36 (m, 2H), 7.77 (d, J=4.50 Hz, 1H), 7.88 (s, 1H), 7.98 (s, 1H), 8.24 (d, J=6.00 Hz, 1H), 8.53 (d, J=4.80 Hz, 1H), 8.64 (d, J=5.40 Hz, 1H), 9.31 (s, 1H). MS m/z 432.2 (M+1).

### Example 3

### Synthesis of 6-(3-fluorophenyl)-N-((6-(2-methylpyridin-4-yl)pyridin-3-yl)methyl)isoquinolin-1-amine (Compound No. 3)

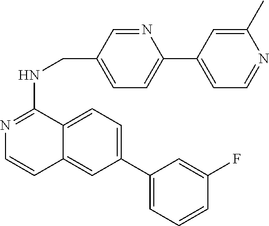 [SMILES: Cc1cc(-c2ccc(CNc3nccc4cc(-c5cccc(F)c5)ccc34)cn2)ccn1]

Step 1:

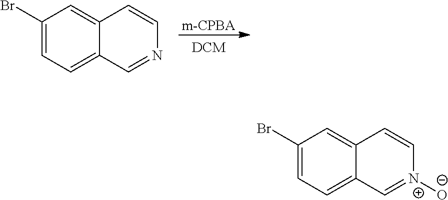

6-bromoisoquinoline (1.80 g, 8.66 mmol) was dissolved in DCM (40 mL), after cooling down the reaction to 0° C. m-CPBA (2.30 g, 1.3 eq, 77% max) was added slowly in small portion. The reaction was warmed up to RT to become a kind of white suspension. In 4 hours, 100 mL DCM was added into the solution, and washed with saturated Na₂CO₃ solution, water and brine. The separated organic layer was dried over Na₂SO₄ and removed under the vacuum to get the yellow solid N-oxide 6-bromoisoquinoline without further purification (1.82 g, yield ˜93%).

Step 2:

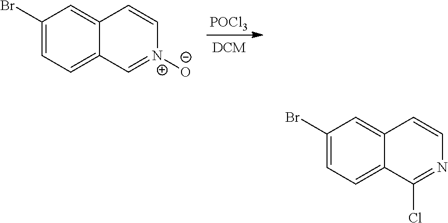

N-oxide 6-bromoisoquinoline (1.82 g, 8.12 mmol) was dissolved in dry DCM (80 mL), POCl₃ (1.12 ml, 1.5 eq) was added dropwise at RT. The reaction was heated to 45° C. for 2 hours. After cooling down the reaction to RT, DCM and excessive POCl₃ were removed under the vacuum. The crude was re-dissolved into 100 mL DCM and was washed by saturated Na₂CO₃, water and brine. The separated organic layer was dried over Na₂SO₄, and concentrated to give brown solid. The crude was purified by flash column using 2% MeOH in DCM to get the pale yellow solid 6-bromo-1-chloroisoquinoline (1.27 g, yield ˜65%). MS m/z 242.0 (M+1).

Step 3:

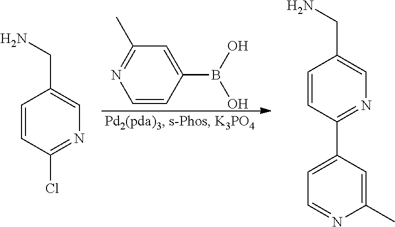

(6-chloropyridin-3-yl)methanamine (300 mg, 2.1 mmol) and 2-methylpyridin-4-ylboronic acid (345 mg, 2.52 mmol) were dissolved in a pressure tube with n-butanol (10 mL) and water (2 mL). K₃PO₄ (893 mg, 4.2 mmol), Pd₂(dba)₃ (96.3 mg, 0.105 mmol), and S-phos (86.4 mg, 0.21 mmol) were added under the nitrogen protection. The reaction was heated to 125° C. for 30 minutes and then cooled down to room temperature. The solution was pull in water and extracted by EA for three times. The combined organic layer was washed by brine and dried over Na₂SO₄, and concentrated under the vacuum. The crude was further purified by flash chromatography with 10% MeOH (containing ˜2N NH) in DCM to get the pure (6-(2-methylpyridin-4-yl)pyridin-3-yl)methanamine (0.19 g, yield ˜45%). MS m/z 200.1 (M+1).

Step 4:

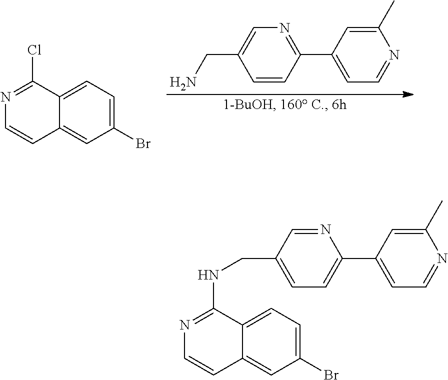

6-bromo-1-chloroisoquinoline (100 mg, 0.41 mmol) and (6-(2-methylpyridin-4-yl)pyridin-3-yl)methanamine (165 mg, 0.82 mmol) were dissolved in 0.5 mL n-BuOH in a sealed tube. The reaction was heat up to 160° C. for 6 h and cooled down to RT. The crude was purified by flash chromatography using 8% MeOH (containing ˜2N NH₃) in DCM to get the pure 6-bromo-N-((6-(2-methylpyridin-4-yl)pyridin-3-yl)methyl)isoquinolin-1-amine (116 mg, ˜70%). MS m/z 405.2 (M+1).

Step 5:

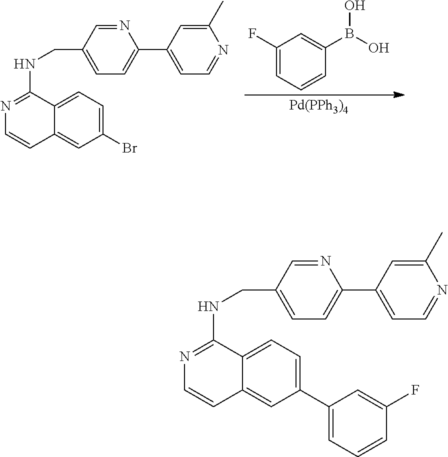

6-bromo-N-((6-(2-methylpyridin-4-yl)pyridin-3-yl)methyl)isoquinolin-1-amine (20 mg, 0.05 mmol), 3-fluorophenylboronic acid (10.5 mg, 0.075 mmol), Na₂CO₃ (21 mg, 0.2 mmol) and Tetrakis(triphenylphosphine)palladium (5.8 mg, 0.005 mmol) were added in a pressure tube. Dioxane/water (3:1, 2 mL) was added into the tube and heated to 125° C. for 10 minutes. After cooling down the reaction to RT, the solution was diluted by 50 mL water and extracted by EA for 3 times. The combined organic layer was dried over Na₂SO₄, and concentrated under the vacuum. The crude was further purified by flash chromatography with 10% MeOH (containing ˜2N NH₃) in DCM to get the pure 6-(3-fluorophenyl)-N-((6-(2-methylpyridin-4-ylpyridin-3-yl)methyl)isoquinolin-1-amine (15.8 mg, ˜75%). 1H NMR (400 MHz, CDCl₃): δ2.71 (s, 3H), 5.00 (d, J=5.6 Hz, 2H), 7.32-7.38 (m, 2H), 7.59-7.65 (m, 1H), 7.75-7.83 (m, 3H), 8.10 (d, J=8.4 Hz, 1H), 8.21 (d, J=8.8 Hz, 1H), 8.27-8.31 (m, 2H), 8.39 (s, 2H), 8.72 (d, J=8.8 Hz, 1H), 8.79 (d, J=6.0 Hz, 1H), 8.91 (d, J=1.6 Hz, 1H), 10.02 (s, 1H). MS m/z 421.2 (M+1).

### Example 4

### Synthesis of N-(4-(2-methylpyridin-4-yl)benzyl)-2-(2-methylpyridin-4-yl)-1,6-naphthyridin-5-amine (Compound No. 4)

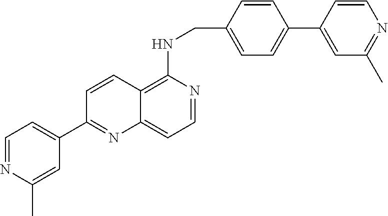 [SMILES: Cc1cc(-c2ccc(CNc3nccc4nc(-c5ccnc(C)c5)ccc34)cc2)ccn1]

Step 1:

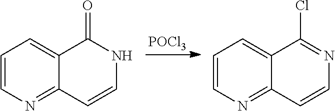

1,6-naphthyridin-5(6H)-one (2.9 g, 19.84 mmol) was dissolved in POCl₃ (40 mL) and heated up to 100° C. for 24 h. After cooling down the reaction to room temperature, the excessive POCl₃ was removed under the vacuum. Small amount crushed ice in saturated Na₂CO₃ solution was added slowly, and lots of bubbles and solid came out. The solid was filtered, and the solution was extracted by EA for 3 times. The combined organic layer was dried over Na₂SO₄, and concentrated under the vacuum. The combined solid was further dried under the vacuum to get 5-chloro-1,6-naphthyridine without further purification (2.6 g, yield ˜80%). MS m/z 165.1 (M+1).

Step 2:

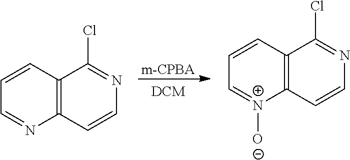

5-chloro-1,6-naphthyridine (1.5 g, 9.11 mmol) was dissolved in DCM (45 mL) and cooled down by ice bath, m-CPBA (3.7 g, 2 eq, 77% max) was added in small portion and slowly. The reaction was warmed up to RT and continued for 3 h. 100 mL more DCM was added into the solution, and washed with saturated Na₂CO₃ solution, water and brine. The organic layer was dried over Na₂SO₄, and concentrated under the vacuum to get yellow solid N-oxide 5-chloro-1,6-naphthyridine without further purification (1.25 g, yield ˜76%).

Step 3:

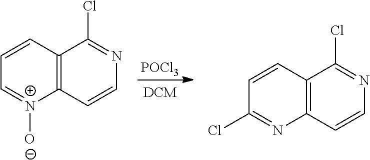

N-oxide 5-chloro-1,6-naphthyridine (1.2 g, 6.64 mmol) was dissolved in dry DCM (30 mL), Et3N (1.85 mL, 13.29 mmol) was added and followed by dropwise adding POCl₃ (0.93 mL, 9.97 mmol) in 5 mL dry DCM. The reaction was heated to 48° C. for 2 h. 100 mL more DCM was added into the solution, and washed with saturated Na₂CO solution, water and brine. The organic layer was dried over Na₂SO₄, and concentrated under the vacuum to get the yellow solid. The crude was further purified by silicon column using EA/hexane (1:4) to get white solid 2,5-dichloro-1,6-naphthyridine (0.6 g, yield ˜45%). MS m/z 199.0 (M+1)

Step 4:

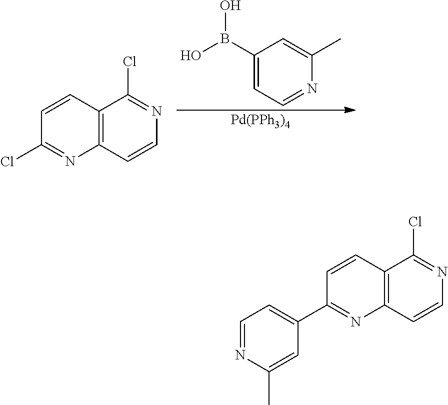

2,5-dichloro-1,6-naphthyridine (200 mg, 1.0 mmol), 2-methylpyridin-4-yl-4-boronic acid (137 mg, 1.0 mmol), Na₂CO₃ (424 mg, 4.0 mmol) and Tetrakis(triphenylphosphine) palladium (116 mg, 0.1 mmol) were added in a flask, dioxane 16 mL and water 4 mL were further added. The reaction was stirred very well and heated to 90° C. for 4 h. After cooling down the reaction to RT, the solution was diluted by 100 mL water and extracted by EA for 3 times. The combined organic layer was dried over Na₂SO₄, and concentrated under the vacuum. The crude was further purified by flash chromatography with EA/hexane (1:1) to get the solid 5-chloro-2-(2-methylpyridin-4-yl)-1,6-naphthyridine (143 mg, yield ˜56%). MS m/z 256.1 (M+1)

Step 5:

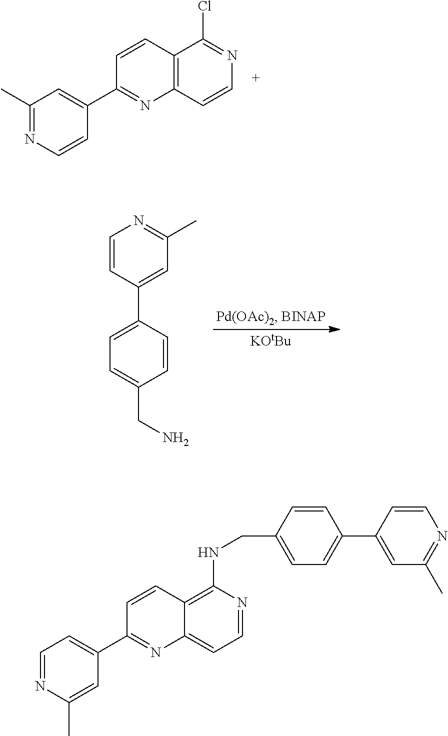

5-chloro-2-(2-methylpyridin-4-yl)-1,6-naphthyridine (20.00 mg, 0.078 mmol) and (4-(2-methylpyridin-4-yl)phenyl)methanamine (25 mg, 0.118 mmol) were dissolved in toluene (2.0 mL). KOtBu (13.2 mg, 0.118 mmol), Pd(OAc)₂ (2.7 mg, 0.012 mmol) and BINAP (15.0 mg, 0.024 mmol) were added into the mixture under N₂. The reaction was heated up to 100° C. overnight. After cooling down the reaction to RT, poured the mixture into the water, extracted by EA for three times. The combined organic layer was washed with brine, dried over Na₂SO₄, then concentrated under vacuum. The crude product was purified by prep-TLC by 8% MeOH in DCM to N-(4-(2-methylpyridin-4-yl)benzyl)-2-(2-methylpyridin-4-yl)-1,6-naphthyridin-5-amine (31 mg, yield ˜61%). ¹H NMR (400 MHz, DMSO-d6): δ9.12 (d, J=8.8 Hz, 1H), 8.77-8.83 (m, 2H), 8.49 (d, J=8.4 Hz, 1H), 8.40 (s, 1H), 8.31 (d, J=6.4 Hz, 1H), 8.21 (s, 1H), 8.11 (d, J=5.6 Hz, 1H), 8.06 (d, J=6.4 Hz, 1H), 7.99 (d, J=8.4 Hz, 2H), 7.65 (d, J=8.4 Hz, 2H), 7.23 (d, J=6.4 Hz, 1H), 5.76 (s, 1H), 4.93 (d, J=5.6 Hz, 2H), 2.72 (s, 6H). MS m/z 432.2 (M+1).

### Example 5

### Synthesis of N-(4-(2-methylpyridin-4-yl)benzyl)-2-phenylpyrido[4,3-b]pyrazin-5-amine (Compound No. 5)

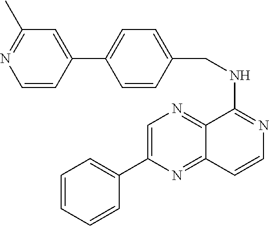 [SMILES: Cc1cc(-c2ccc(CNc3nccc4nc(-c5ccccc5)cnc34)cc2)ccn1]

Step 1:

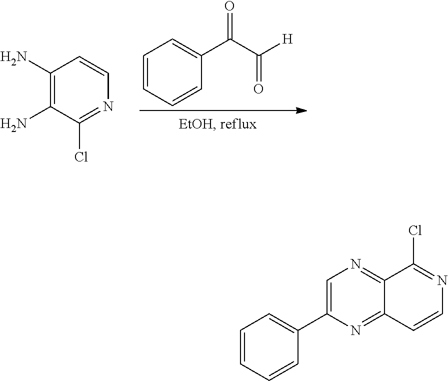

To 20 mL of ethanol was added phenyl gloyoxal monohydrate (940 mg, 6.99 mmol) and 2-chloro-3,4-diaminopyridine (1000 mg, 6.99 mmol). The mixture was refluxed for overnight. After cooling down the reaction, the crude precipitated product was filtered and washed with 15 mL ethanol and dried under vacuum to get 5-chloro-2-phenylpyrido[3,4-b]pyrazine without further purification (1.28 g, yield ˜76%), MS m/z 241.0 (M+1); 1H NMR (300 MHz, DMSO-d6): δ 9.82 (s, 1H), 8.64 (d, J=6.0 Hz, 1H), 8.38-8.43 (m, 2H), 8.07 (d, J=6.0 Hz, 1H), 7.64-7.68 (m, 3H).

Step 2:

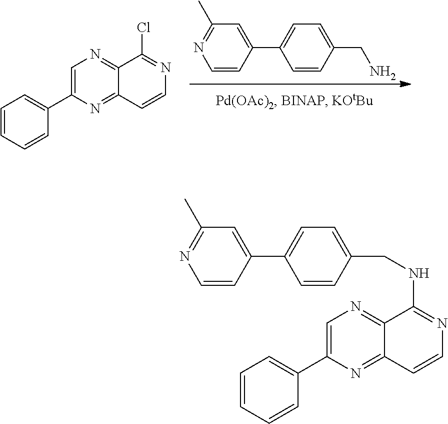

N-(4-(2-methylpyridin-4-yl)benzyl)-2-phenylpyrido[3,4-b]pyrazin-5-amine (50 mg, 0.21 mmol) and (4-(2-methylpyridin-4-yl)phenyl)methanamine (42 mg, 0.21 mmol) were dissolved in toluene (4.0 mL). KOtBu (24 mg, 0.21 mmol), Pd(OAc)₂ (4.5 mg, 0.021 mmol) and BINAP (26.4 mg, 0.042 mmol) was added into the mixture under N₂. The reaction was heated up to 100° C. for overnight. After cooling down the reaction to RT, poured the mixture into the water, extracted by EA for three times. The combined organic layer was washed with brine, dried over Na₂SO₄, then concentrated under vacuum. The crude product was purified by flash chromatography using 7% MeOH in DCM to get N-(4-(2-methylpyridin-4-yl)benzy-2-phenylpyrido[4,3-b]pyrazin-5-amine (61 mg, yield ˜72%). MS m/z=404.2 (M+1); ¹H NMR (400 MHz, DMSO-d6) δ 9.53 (s, 1H), 8.77 (d, J=6.4 Hz, 1H), 8.35-8.39 (m, 2H), 8.21 (s, 1H), 8.11 (d, J=6.0 Hz, 1H), 8.07 (d, J=6.4 Hz, 1H), 7.96 (d, J=8.4 Hz, 2H), 7.60-7.65 (m, 5H), 7.14 (d, J=6.0 Hz, 1H), 5.76 (s, 1H), 4.90 (d, J=6.4 Hz, 2H), 2.71 (s, 3H).

### Example 6

RNA Extraction and SYBR Green Real-Time RT-PCR

Total RNA was isolated from frozen tumor tissue using RNAeasy extraction kit (QIAGEN) according to the manufacturers instructions and reverse transcribed into 1^{st} cDNA. Rspo2 forward primer 5′-AGAGGCCGTTGCTTTGATGA-3′ (SEQ ID NO.:1) and reverse primer 5′-TCCCCATTCGCTCCAATGAC-3′ (SEQ ID NO.:2). GAPDH forward primer 5′-GAAGGTGAAGGTCGGAGT-3′ (SEQ ID NO.:3) and reverse primer 5′-GAAGATGGTGATGGGATTTC-3′ (SEQ ID NO.:4). 1:200 diluted cDNA template was used for GAPDH amplification. The PCR amplification profile for Rspo2, Rspo3 and GAPDH was one cycle of 10 min at 94° C. followed by 40 cycles in two steps consisting of 15 second at 94° C. and 1 min at 60° C. The fluorescence intensity of the products was measured at the end of each cycle and post-PCR melt curve analysis was performed to detect primer-dimers or other non-specific products and to confirm the specificity of the target. Amplification, data acquisition and analysis were carried out using an Applied Biosystems 7500 Real-Time PCR instrument (Life Technologies, Foster City, Calif.). Three replicates of each sample with specific primers were performed in 96-well plate along with positive control and negative control. The positive and negative controls were total RNAs from tumor tissues, in which Rspo2 and Rspo3 expression was previously characterized. The relative of Rspo2 and Rspo3 was determine by ΔCt, where ΔCt=Ct (Rspo2 or Rspo3)−Ct (GAPDH).

5′ RACE, Cloning and Sequencing

Total RNA was used to amplify the 5′end of the human Rspo2 or Rspo3 mRNA using the SMARTer® RACE 5′/3′ kit (Clontech Laboratories, Mountain View, Calif.) according to the manufacturers instructions.

The Rspo2 exon 2 gene specific primer

(SEQ ID NO.: 5)
5′-GATTACGCCAAGCTTCGTCTCCATCGGTTGCCTTGGCAGTGGC-3′,

exon3 gene specific primer 5′-GATTACGCCAAGCTTGCAGGCACTCTCCATACTGGCGCATCCC-3′ (SEQ ID NO.:6), exon 3 nested primer

(SEQ ID NO.: 7)
5′-GATTACGCCAAGCTTGGGCTCGGTGTCCATAGTACCCGGATGGG-3′.

The Rspo3 exon 3 gene specific prime

(SEQ ID NO.: 8)
5′-GATTACGCCAAGCTTGGTTGTTGGCTTCCAACCCTTCTGGGC-3′

and exon3 nested gene specific prime

(SEQ ID NO.: 9)
5′-GATTACGCCAAGCTTGGACCCGTGTTTCAGTCCCTCTTTTGAAGCC-3′.

15nt in-fusion cloning primer (underlined) was included at the 5′ end for RACE product cloning using the SMARTer® RACE 5′/3′ kit (Clontech Laboratories, Mountain View, Calif.).

The 5′ RACE products were cloned into the In-Fusion vector. The insert of 10 clones was sequenced by the M13 primer and analyzed by NCBI nucleotide BLAST (http://blast.ncbi.nlm.nih.gov/Blast.cgi).

Nanostring nCounter Element Chemistry Technology

nCounter assays were performed with customized designed Element chemistry probes according to the manufacturers protocol (NanoString, Seattle, Wash.). Briefly, 150 ng of total RNA was hybridized to nCounter probe sets for 17.75 hours at 67° C. and ramp down to 4° C. for about 3 h. Samples were processed using an automated nCounter Sample Prep Station (NanoString Technologies, Inc., Seattle, Wash.). Cartridges containing immobilized and aligned reporter complex were subsequently imaged on an nCounter Digital Analyzer (NanoString Technologies, Inc.). Reporter counts were collected using NanoString's nSOlver analysis software 2.0, normalized, and analyzed with positive controls and housekeeping genes.

TABLE 2
Nanostring Element chemistry probe A (capture probe)

| | | SEQ ID |
|---|---|---|
| Name | Sequence | NO. |
| NM_001101.2: | GATCTTGATCTTCATTGTGCTGGGTGCCAGGGCAGTGATCT | 10 |
| 1010_T001 | CCTTCTGCACCTCAAGACCTAAGCGACAGCGTGACCTTGTT | |
| | TCA | |
| NM_002046.3: | AAGTGGTCGTTGAGGGCAATGCCAGCCCCAGCGTCAAAGC | 11 |
| 972_T002 | ATCCTCTTCTTTTCTTGGTGTTGAGAAGATGCTC | |
| NM_004655.3: | TCGGAACAGGTAAGCACCGTCTTGATCGCCCAATAAGGAG | 12 |
| 527_T003 | TGTAAGGACTCACAATTCTGCGGGTTAGCAGGAAGGTTAG | |
| | GGAAC | |
| NM_003667.3: | GAAAACCCTTATTTTCCTAATTCCCCCAACAACCTCTTGAGT | 13 |
| 3414_T004 | TTGTGGGTCTGTTGAGATTATTGAGCTTCATCATGACCAGA | |
| | AG | |
| NM_178565.4: | TTCCTGCGCTTTCTCCACGGTCACTTCACAGCTAAGATTTC | 14 |
| 150_T005 | TTTCAAAGACGCCTATCTTCCAGTTTGATCGGGAAACT | |
| NM_178565.4: | GATGAGGGCAAAGGAGAAAAGGCGAAACTGCATCTGGGCG | 15 |
| 641_T006 | GTCGGGCGGGCGAACCTAACTCCTCGCTACATTCCTATTGT | |
| | TTTC | |
| NM_178565.4: | CTCCAATGACCAACTTCACATCCTTCCACACATTCCATGGTT | 16 |
| 1060_T007 | TCTTCTAACCAATTTGGTTTTACTCCCCTCGATTATGCGGAG | |
| | T | |
| NM_178565.4: | GTTGTGTTTCATCAAAGTCATCACATCTTAGACTCAAAGGCA | 17 |
| 2289_T008 | GTTTGGGCCTTTCGGGTTATATCTATCATTTACTTGACACCC | |
| | T | |
| Fusion_ | CCTCCTTTACAGAGAGAAATTCAAGAAGCGGAAAGACTAGA | 18 |
| 0622.1:69_T009 | TGCCGATCCCAACAGCCACTTTTTTTCCAAATTTTGCAAGA | |
| | GCC | |
| Fusion_ | CCACGAACCTTCCCAAGTGACATCGTAAAGCTCTGAGACCT | 19 |
| 0623.1:33_T010 | TCGCCATCTCACCGTGTGGACGGCAACTCAGAGATAACGC | |
| | ATAT | |
| Fusion_ | AAGGCTGTGGTAGGGAACGGAGTCTATATTTGTAGAGAAG | 20 |
| 0625.1:77_T011 | AGTAGAATCACCTGGAGTTTATGTATTGCCAACGAGTTTGT | |
| | CTTT | |
| Fusion_ | AACTGGAGGGCAGATCTGGCCGTGTCTCCACAGGTCAGAT | 21 |
| 0624.1:155_T012 | AAGGTTGTTATTGTGGAGGATGTTACTACA | |
| NM_032784.4: | TAAAATATATGTAGGTGTTAGGCGTATATAGACAGTGCCCG | 22 |
| 173_T013 | AGCAGCGGGCTTCCTTCCTGTGTTCCAGCTACAAACTTAGA | |
| | AAC | |
| NM_032784.4: | CTTCTGGGCAATTGTCAAGGCACTTTCCAAGGTGTAAGTAA | 23 |
| 634_T014 | AATCCACTTCATAAAATTGGTTTTGCCTTTCAGCAATTCAAC | |
| | TT | |
| NM_032784.4: | CTCCCGGTCAAAACAACACTGTCCTTTGAAGGATGTTTCTC | 24 |
| 1520_T015 | TTCCCTGAACTGGTCAAGACTTGCATGAGGACCCGCAAATT | |
| | CCT | |
| Fusion_ | CTTGGGGCATCTCGGGTGGTAGATAATGAGGCTCTTGAGC | 25 |
| 0169.1:1_T016 | ACTAACATGCTTTCGTTGGGACGCTTGAAGCGCAAGTAGAA | |
| | AAC | |
| Fusion_ | CACAAGGATAACTCAGTACTTGGATGTCATCAATGGCAATA | 26 |
| 0736.1:0_T017 | TAACCACTTCCAGCAGACCTGCAATATCAAAGTTATAAGCG | |
| | CGT | |
| Fusion_ | CTCTCACAATAAGTTGAGCAAAATTGGACACACCGGAACCT | 27 |
| 0170.1:1_T018 | CGTTCTGACCTGCCAATGCACTCGATCTTGTCATTTTTTTGC | |
| | G | |
| Fusion_ | CATCTTCATCAGTTTGAATAATTGTCTCTTCACTCTCCTTCC | 28 |
| 0737.1:0_T0719 | TTCCCTCCAAACTGGAGAGAGAAGTGAAGACGATTTAACCC | |
| | A | |
| Fusion_ | CTGCGGAGAACTGGCCTTGGGCCGATCCCAGGAGACGATT | 29 |
| 0524.1:0_T020 | GCTGCATTCCGCTCAACGCTTGAGGAAGTA | |
| Fusion_ | CTGCACATTTTGTTCTGGTGATTAGTGGAGGTCCTGGGAGC | 30 |
| 0525.1:0_T021 | TGAGGCTGTTAAAGCTGTAGCAACTCTTCCACGA | |
| Fusion_ | CTTTTGTAGCAATGCGTACGCACTGGGTTTTAGTTTCCTTCT | 31 |
| 0526.1:0_T022 | CCACACTGCTAGGACGCAAATCACTTGAAGAAGTGAAAGC | |
| | GAG | |
| Fusion_ | CTCCTTTTCACTTGTCATTTCAGTAACTGCAGGCCTTTTCTG | 32 |
| 0684.1:0_T023 | CGGAGAACCCACGCGATGACGTTCGTCAAGAGTCGCATAA | |
| | TCT | |
| Fusion_ | CTGAGATATTGGTGGTGACATTGATGGCTGTGGCTGGACC | 33 |
| 0738.1:0_T024 | AAAGCCTTTGCATTTGGAATGATGTGTACTGGGAATAAGAC | |
| | GACG | |

TABLE 3
Nanostring Element Chemistry Probe B design (reporter probe)

| | | SEQ ID |
|---|---|---|
| Name | Sequence | NO. |
| NM_001101.2: | CGAAAGCCATGACCTCCGATCACTCAGGATGGAGCCGCCG | 34 |
| 1010_ProbeB | ATCCACACGGAGTACTTGCGCTCAGGAGGAGCAAT | |
| NM_002046.3: | CGAAAGCCATGACCTCCGATCACTCCCCTGTTGCTGTAGC | 35 |
| 972_ProbeB | CAAATTCGTTGTCATACCAGGAAATGAGCTTGACA | |
| NM_004655.3: | CGAAAGCCATGACCTCCGATCACTCGCAAACCAGAAGTCT | 36 |
| 527_ProbeB | AAGGTATCCACGCATTTCTCCCTCTCCAGGAAAGT | |
| NM_003667.3: | CGAAAGCCATGACCTCCGATCACTCTAGAATGAAATCCCAT | 37 |
| 3414_ProbeB | GGATCACAGCCTCTACCTAGCAATGTAGGTCATT | |
| NM_178565.4: | CGAAAGCCATGACCTCCGATCACTCCGCTCACACTCTCTG | 38 |
| 150_ProbeB | CCTCCCTAACCAATTGTGTCGC | |
| NM_178565.4: | CGAAAGCCATGACCTCCGATCACTCATCGGTTGCCTTGGC | 39 |
| 641_ProbeB | AGTGGCTGTAATCCATGCAGTTCAGAAT | |
| NM_178565.4: | CGAAAGCCATGACCTCCGATCACTCCCCATTTAAATCCACA | 40 |
| 1060_ProbeB | TGTGCGATTATTTCTGCTACAAGTTCCCCATTCG | |
| NM_178565.4: | CGAAAGCCATGACCTCCGATCACTCGGCCTGTGAAACTGG | 41 |
| 2289_ProbeB | CTACAAGTGACTGGATATAGTCCCTCATGTTTTCA | |
| Fusion_ | CGAAAGCCATGACCTCCGATCACTCCGGTCAGTTCAGCGC | 42 |
| 0622.1:69_ProbeB | GATCAGCATCTCTCCGCCACGAA | |
| Fusion_ | CGAAAGCCATGACCTCCGATCACTCCGCACCGGTCAGTTC | 43 |
| 0623.1:33_ProbeB | AGCGCGATCAGCATCTCTCCG | |
| Fusion_ | CGAAAGCCATGACCTCCGATCACTCCGGTCAGTTCAGCGC | 44 |
| 0625.1:77_ProbeB | GATCAGCATCTCTCCGCCACGAAC | |
| Fusion_ | CGAAAGCCATGACCTCCGATCACTCCGGTCAGTTCAGCGC | 45 |
| 0624.1:155_ProbeB | GATCAGCATCTCTCCGCCACG | |
| NM_032784.4: | CGAAAGCCATGACCTCCGATCACTCTCCCTTCCTTTCTCCT | 46 |
| 173_ProbeB | CTTTCTTTTGATTGTTAATTATATTTAATGTTTT | |
| NM_032784.4: | CGAAAGCCATGACCTCCGATCACTCACAGTGCACAATACT | 47 |
| 634_ProbeB | GACACACTCCATAGTATGGTTGTTGGCTTCCAACC | |
| NM_032784.4: | CGAAAGCCATGACCTCCGATCACTCAAATCCTGTGATTCCA | 48 |
| 1520_ProbeB | AATGCCAGGCCCTAATTCTGAGCACTCTCTAGAT | |
| Fusion_ | CGAAAGCCATGACCTCCGATCACTCCACAGCCTCCTTGGC | 49 |
| 0169.1:1_ProbeB | AGCCTTGACTAACGTTAGGATGCA | |
| Fusion_ | CGAAAGCCATGACCTCCGATCACTCCACAGCCTCCTTGGC | 50 |
| 0736.1:0_ProbeB | AGCCTTGACTAACGTTAGGATGCA | |
| Fusion_ | CGAAAGCCATGACCTCCGATCACTCCACAGCCTCCTTGGC | 51 |
| 0170.1:1_ProbeB | AGCCTTGACTAACGTTAGGATGCA | |
| Fusion_ | CGAAAGCCATGACCTCCGATCACTCCACAGCCTCCTTGGC | 52 |
| 0737.1:0_ProbeB | AGCCTTGACTAACGTTAGGATGCA | |
| Fusion_ | CGAAAGCCATGACCTCCGATCACTCCACAGCCTCCTTGGC | 53 |
| 0524.1:0_ProbeB | AGCCTTGACTAACGTTAGGATGCA | |
| Fusion_ | CGAAAGCCATGACCTCCGATCACTCCACAGCCTCCTTGGC | 54 |
| 0525.1:0_ProbeB | AGCCTTGACTAACGTTAGGATGCA | |
| Fusion_ | CGAAAGCCATGACCTCCGATCACTCCACAGCCTCCTTGGC | 55 |
| 0526.1:0_ProbeB | AGCCTTGACTAACGTTAGGATGCA | |
| Fusion_ | CGAAAGCCATGACCTCCGATCACTCCACAGCCTCCTTGGC | 56 |
| 0684.1:0_ProbeB | AGCCTTGACTAACGTTAGGATGCA | |
| Fusion_ | CGAAAGCCATGACCTCCGATCACTCCACAGCCTCCTTGGC | 57 |
| 0738.1:0_ProbeB | AGCCTTGACTAACGTTAGGATGCA | |

Increased Expression of Rspo2 and Rsp3 by their 5′ Fusion Genes

The amount of Rspo2 and Rspo3 transcripts increased in about 5% and 8% of tumor samples (n=192). 5′ RACE and DNA sequencing identified that the up-regulation of Rspo2 and Rspo3 transcripts were driven by its 5′ fusion gene expression.

TABLE 4
Real-time RT-PCR quantifying Rspo2 gene expression

| Sample | GAPDH | RSPO2 | ΔCT |
|---|---|---|---|
| GAPF108 | 23.2 | 38.4 | 15.3 |
| ESPF001 | 21.9 | 36.5 | 14.5 |
| LUPF016 | 25.6 | 40.0 | 14.4 |
| LIPF236 | 22.8 | 26.3 | 3.6 |
| PAPF179 | 23.2 | 26.8 | 3.5 |
| LIPF088 | 21.5 | 25.0 | 3.5 |
| PAPF004 | 21.2 | 23.7 | 2.4 |
| PAPF310 | 21.9 | 24.2 | 2.2 |
| ESPF014 | 22.2 | 23.4 | 1.2 |
| PAPF199 | 24.2 | 25.4 | 1.1 |
| PAPF029 | 22.5 | 22.6 | 0.1 |
| PAPF157 | 26.8 | 26.9 | 0.1 |
| GAPF3055 | 23.7 | 23.5 | −0.2 |
| GAPF67 | 22.4 | 19.7 | −2.7 |
| CRPF3056 | 23.4 | 19.7 | −3.7 |
| CRPF2506 | 27.6 | 23.6 | −4.0 |
| GLPF0440 | 26.6 | 21.2 | −5.4 |

Identified Novel Rspo2 and Rspo3 Transcripts

Various novel Rspo2 and Rspo3 gene fusion transcripts have been identified.

TABLE 5
Novel Rspo2 and Rspo3 fusion genes

| 5′ gene | | Rspo2 | in-frame |
|---|---|---|---|
| EMC2 | exon1 | exon2 | yes |
| PVT1 | exon1 | exon2 | yes |
| PVT1 | exon1 | exon2 | no |
| HNF4G | | exon2 | yes |
| 5′ gene | | Rspo3 | in-frame |
| PTPRK | Exon6x | Exon2 | yes |
| PTPRK | exon13 | exon2 | yes |

Characterization of the Fusion Genes by Nanostring nCounter Assay

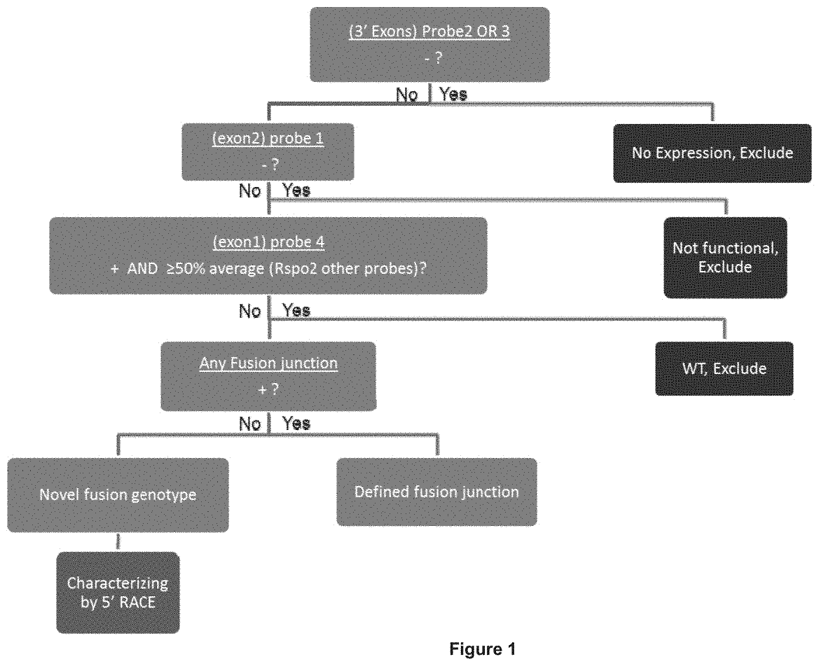

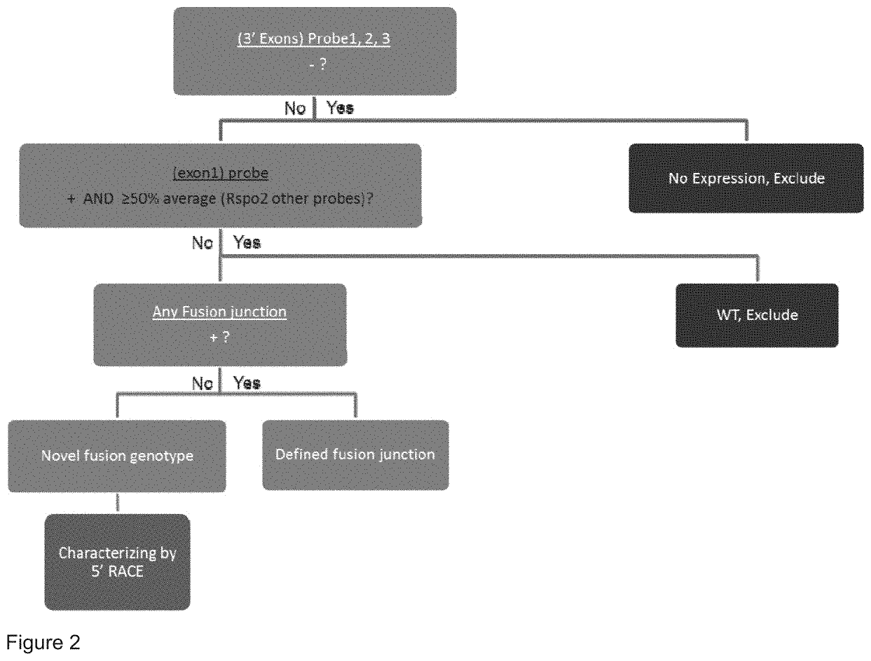

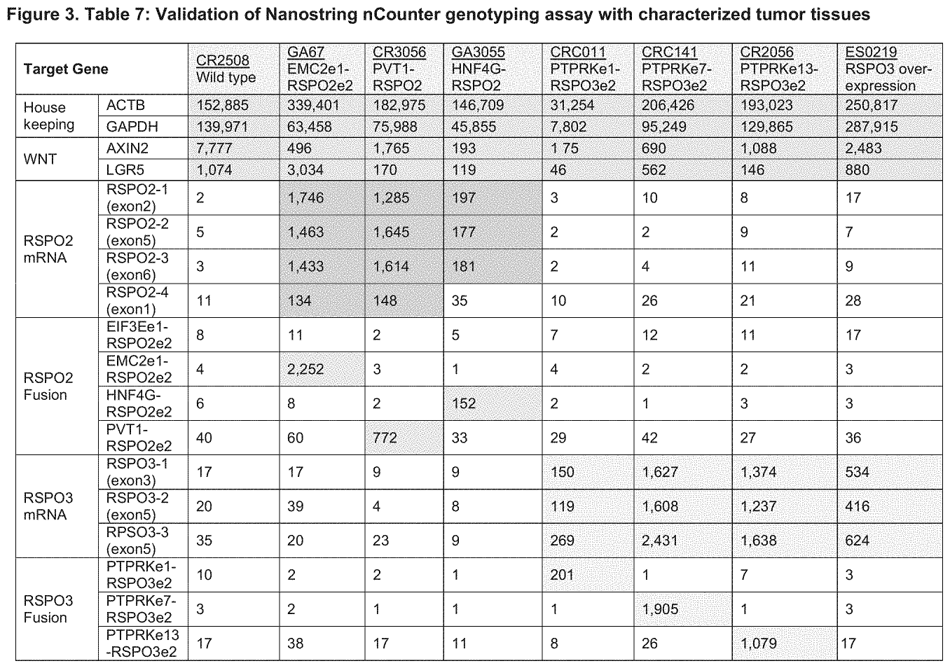

Tumor tissues carrying Rspo2 or Rspo3 fusion genes were used to validate Nanostring nCounter genotyping assay (FIG. 3, Table 7). Fusion junction probes were specifically designed targeting the fusion genotypes characterized by 5′ RACE and sequencing. The decision making steps on known or novel Rspo2/Rspo3 fusion genotypes were illustrated in FIG. 1 and FIG. 2.

The Rspo2 expression was assessed by the probes targeting exon 2, exon 5 and exon 6. The start codon ATG resides in exon 2, producing full length rspo2 protein from the fusion genes. Rspo2 exon1 was not observed in any Rspo2 fusion genotypes, but found present only in wild type Rspo2 transcripts.

The Rspo3 expression was assessed by probes targeting exon3/4 and exon5. Open reading frame Rspo3 depends on the in-frame sequence of its 5′ fusion genes.

Five tumor tissues with characterized novel Rspo2 fusion genotypes and one sample with Rspo3 fusion genotypes were correctly identified with their fusion junctions by Nanostring nCounter genotyping assay. Correspondingly, the Rspo2 or Rspo3 expression was observed in these samples. Increased Rspo2 signal of L440 was found in exon1, 2, 5 and 6 probes. 5′ RACE and sequencing identified that L440 predominantly carries complete Rspo2 mRNA, and its expression was not driven by fusion genes.

TABLE 6
Probe design for Rspo2 and Rspo3 fusion genotyping

| Sample Date | Probes |
|---|---|
| Housekeeping | ACTB |
| | GAPDH |
| Rspo2 | RSPO2-1(exon1) |
| Expression | RSPO2-2a (exon2) |
| Rspo2 Fusion | RSPO2-3 (exon5) |
| junction | RSPO2-4 (exon6) |
| | EIF3Eex1-RSPO2ex2 |
| | EIF3Eex1-RSPO2ex3 |
| | EMC2ex1-RSPO2ex2 |
| | EMC2ex1-RSPO2ex3 |
| | HNF4G-RSPO2 |
| | PVT1-RSPO2ex2 |
| | PVT1-RSPO2ex3 |
| Rspo3 | RSPO3-1 (exon1) |
| Expression | RPSO3-2 (exon3/4) |
| Rspo3 Fusion | RPSO3-3 (exon5) |
| junction | PTPRKe1-RSPO3e2 |
| | PTPRKe13-RSPO3e2 |
| | PTPRKe7-RSPO3e2 |
| | PTPRKe6X-RSPO3e2 |

PDx Efficacy Study on Rspo2/3 Fusion Models

The anti-tumor activity of CGX1321 was examined in the colorectal and gastric tumors with Rspo2 or Rspo3 fusion genes in mouse xenograft models. BALB/c nude mice at the age of 8-10 weeks old were inoculated subcutaneously on the right flank with a tumor fragment of 2×2×2 mm for tumor development. Tumor development was allowed undisrupted until the mean volume reached approximately 100-150 mm³. Mice were then randomized into control and the treatment groups. CGX1321 was administered to the tumor-bearing mice orally for 21-28 days at predetermined regiment. Tumor measurement was conducted twice weekly with a caliper and the tumor volume (mm³) was estimated using the formula: TV=a×b²/2, where a and b are long and short diameters of a tumor, respectively. The body weight was assessed at the same time as the tumor measurement.

What is claimed is:

1. A method for treating cancer characterized by expression of an R-spondin fusion in a subject that has been diagnosed with cancer and is in need of such treatment, comprising:
administering to a subject diagnosed with cancer a pharmaceutical composition comprising a therapeutically effective amount of an antagonist of Porcupine,
wherein said subject's cancer has been determined to have an R-spondin fusion comprising
(1) an HNF4G-Rspo2e2 fusion;
(2) a PTPRKe13-Rspo3e2 fusion; or
(3) a PTPRKe6X-Rspo3e2 fusion;
wherein said Porcupine antagonist comprises:
a compound of Formula (I): 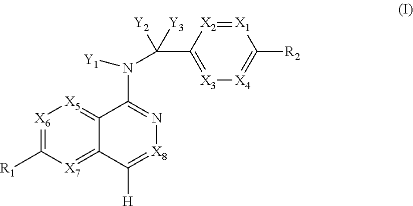
or a physiologically acceptable salt thereof, wherein
X₁, X₂, X₃, X₄, X₅, X₆, X₇, X₈ are independently CR4 or N;
Y₁ is hydrogen or CRa; Y₂, Y₃ are independently hydrogen, halo or CR3;
R₁ is morpholinyl, piperazinyl, quinolinyl, 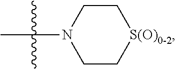
aryl, C₁₋₆ heterocycle, 5 or 6 membered heteroaryl containing 1-2 heteroatoms selected from N, O and S;
R₂ is hydrogen, halo, morpholinyl, piperazinyl, quinolinyl, 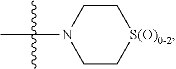
aryl, C₁₋₆ heterocycle, 5 or 6 membered heteroaryl containing 1-2 heteroatoms selected from N, O and S;
R₃ is hydrogen, halo, cyano, C₁₋₆ alkyl, C₁₋₆ alkoxy optionally substituted with halo, amino, hydroxyl, alkoxy or cyano;
R₄ is hydrogen, halo, C₁₋₆alkoxy, —S(O)₂R₅, —C(O)OR₅, —C(O)R₅, —C(O)NR₆R₇, C₁₋₆ alkyl, C₂₋₆ alkenyl or C₂₋₆alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆alkynyl, each of which may be optionally substituted with halo, amino, hydroxyl, alkoxy or cyano;
a compound of Formula (II): 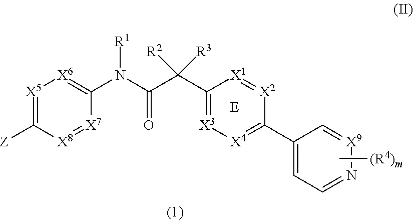
or a physiologically acceptable salt thereof, wherein:
X¹, X², X³ and X⁴ is selected from N and CR⁷;
one of X⁵, X⁶, X⁷ and X⁸ is N and the others are CH;
X⁹ is selected from N and CH;
Z is selected from phenyl, pyrazinyl, pyridinyl, pyridazinyl and piperazinyl;
wherein each phenyl, pyrazinyl, pyridinyl, pyridazinyl or piperazinyl of Z is optionally substituted with an R⁶ group;
R¹, R² and R³ are hydrogen;
m is 1;
R⁴ is selected from hydrogen, halo, difluoromethyl, trifluoromethyl and methyl;
R⁶ is selected from hydrogen, halo and —C(O)Rⁱ⁰; wherein R¹⁰ is methyl; and
R⁷ is selected from hydrogen, halo, cyano, methyl and trifluoromethyl.

2. The method of claim 1, wherein said subject's cancer is determined to have R-spondin mRNA expression level that is higher than the R-spondin mRNA expression level in a control subject that has been determined to not have a R-spondin fusion.

3. The method of claim 1, wherein:
1) said HNF4G-Rspo2e2 fusion comprises a junction sequence of SEQ ID NO.:67;
2) said PTPRKe13-Rspo3e2 fusion comprises a junction sequence of SEQ ID NO.:61; or
3) said PTPRKe6X-Rspo3e2 fusion comprises a junction sequence of SEQ ID NO.:60.

4. The method of claim 1, wherein said R-spondin is Rspo2 or Rspo3, and said fusion gene is overexpressed in comparison to the R-spondin that is not fused to another gene.

5. The method of claim 1, wherein said 5 or 6 membered heteroaryl of R² of Formula (I) is selected from: 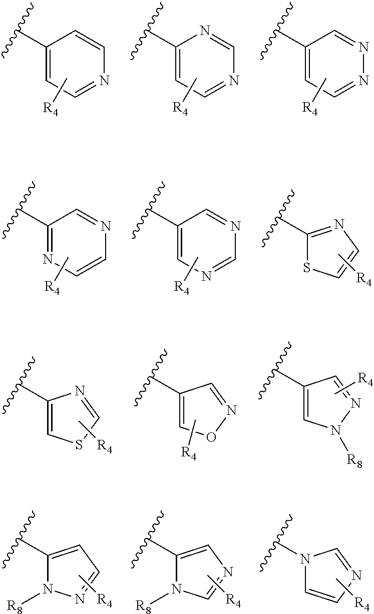
wherein,
R₄ is hydrogen, halo, C₁₋₆alkoxy, —S(O)₂R₅, —C(O)OR₅, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₅, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano; and
R₈ is hydrogen or C₁₋₆ alkyl.

6. The method of claim 1, wherein R₁ and R₂ of Formula (I) is independently substituted with 1 or 2 R₄ groups.

7. The method of claim 1, wherein said compound is:
6-(2-methylpyridin-4-yl)-N-(4-(2-methylpyridin-4-yl)benzyl)-2,7-naphthyridin-1-amine;
N-(3-methyl-4-(2-methylpyridin-4-yl)benzyl)-6-(2-methylpyridin-4-yl)-2,7-naphthyridin-1-amine;
6-(3-fluorophenyl)-N-((2′-methyl-2,4′-bipyridin-5-yl)methyl)isoquinolin-1-amine;
2-(2-methylpyridin-4-yl)-N-(4-(2-methylpyridin-4-yl)benzyl)-1,6-naphthyridin-5-amine;
N-(4-(2-methylpyridin-4-yl)benzyl)-2-phenylpyrido[4,3-b]pyrazin-5-amine;
N-(4-(2-methylpyridin-4-yl)benzyl)-6-(pyridin-4-yl)-2,7-naphthyridin-1-amine;
N-(4-(2-methylpyridin-4-yl)benzyl)-6-phenyl-2,7-naphthyridin-1-amine;
6-(3-chlorophenyl)-N-(4-(2-methylpyridin-4-yl)benzyl)-2,7-naphthyridin-1-amine;
6-(3-fluorophenyl)-N-(4-(2-methylpyridin-4-yl)benzyl)-2,7-naphthyridin-1-amine;
6-(3-fluorophenyl)-N-((2′-methyl-2,4′-bipyridin-5-yl)methyl)-2,7-naphthyridin-1-amine;
6-(3-fluorophenyl)-N-(4-(2-(trifluoromethyl)pyridin-4-yl)benzyl)-2,7-naphthyridin-1-amine;
N-(4(2′,3-dimethyl-2,4′-bipyridin-5-yl)methyl)-6-(3-fluorophenyl)-2,7-naphthyridin-1-amine;
N-(4-(2-methylpyridin-4-yl)benzyl)-6-(pyrimidin-5-yl)-2,7-naphthyridin-1-amine;
6-(5-methylpyridin-3-yl)-N-(4-(2-methylpyridin-4-yl)benzyl)-2,7-naphthyridin-1-amine;
6-(6-methylpyridin-3-yl)-N-(4-(2-methylpyridin-4-yl)benzyl)-2,7-naphthyridin-1-amine;
3-(8-(4-(2-methylpyridin-4-yl)benzylamino)-2,7-naphthyridin-3-yl)benzonitrile;
4-(8-(4-(2-methylpyridin-4-yl)benzylamino)-2,7-naphthyridin-3-yl)benzonitrile;
6-(4-fluorophenyl)-N-(4-(2-methylpyridin-4-yl)benzyl)-2,7-naphthyridin-1-amine;
N-(4-(2-methylpyridin-4-yl)benzyl)-6-m-tolyl-2,7-naphthyridin-1-amine;
N-(4-(2-methylpyridin-4-yl)benzyl)-2,7-naphthyridin-1-amine;
N-(4-(2-methylpyridin-4-yl)benzyl)-6-(pyridin-2-yl)-2,7-naphthyridin-1-amine;
6-(2-fluoropyridin-4-yl)-N-(4-(2-methylpyridin-4-yl)benzyl)-2,7-naphthyridin-1-amine;
6-(2-fluorophenyl)-N-(4-(2-methylpyridin-4-yl)benzyl)-2,7-naphthyridin-1-amine;
N-(4-(2-methylpyridin-4-yl)benzyl)-6-(pyridin-3-yl)-2,7-naphthyridin-1-amine;
N-(biphenyl-4-ylmethyl)-6-(2-methylpyridin-4-yl)-2,7-naphthyridin-1-amine;
6-(2-methylpyridin-4-yl)-N-((5-phenylpyridin-2-yl)methyl)-2,7-naphthyridin-1-amine;
6-(3-fluorophenyl)-N-((2′-(trifluoromethyl)-2,4′-bipyridin-5-yl)methyl)-2,7-naphthyridin-1-amine;
N-(3-fluoro-4-(2-fluoropyridin-4-yl)benzyl)-6-(2-methylpyridin-4-yl)-2,7-naphthyridin-1-amine;
6-(2-methylpyridin-4-yl)-N-((2′-(trifluoromethyl)-2,4′-bipyridin-5-yl)methyl)-2,7-naphthyridin-1-amine;
N-((3-fluoro-2′-(trifluoromethyl)-2,4′-bipyridin-5-yl)methyl)-6-(2-methylpyridin-4-yl)-2,7-naphthyridin-1-amine;
N-(3-fluoro-4-(2-methylpyridin-4-yl)benzyl)-6-(2-methylpyridin-4-yl)-2,7-naphthyridin-1-amine;
N-((4(2′-fluoro-2,4′-bipyridin-5-yl)methyl)-6-(2-methylpyridin-4-yl)-2,7-naphthyridin-1-amine;
4-(5-(((6-(2-methylpyridin-4-yl)-2,7-naphthyridin-1-yl)amino)methyl)pyridine-2-yl)thiomorpholine 1,1-dioxide;
6-(2-methylpyridin-4-yl)-N-(4-(pyridazin-4-yl)benzyl)-2,7-naphthyridin-1-amine;
N-(4-(2-methylpyridin-4-yl)benzyl)-6-(pyrazin-2-yl)-2,7-naphthyridin-1-amine;
N-(4-(2-methylpyridin-4-yl)benzyl)-6-(pyridazin-4-yl)-2,7-naphthyridin-1-amine;
N-(4-(2-methylpyridin-4-yl)benzyl)-6-morpholino-2,7-naphthyridin-1-amine;
6-(4-methylpiperazin-1-yl)-N-(4-(2-methylpyridin-4-yl)benzyl)-2,7-naphthyridin-1-amine;
4-(8-((4-(2-methylpyridin-4-yl)benzyl)amino)-2,7-naphthyridin-3-yl)thiomorpholine 1,1-dioxide;
N-(3-fluoro-4-(2-fluoropyridin-4-yl)benzyl)-6-(3-fluorophenyl)-2,7-naphthyridin-1-amine;
N-(3-fluoro-4-(2-methylpyridin-4-yl)benzyl)-6-(3-fluorophenyl)-2,7-naphthyridin-1-amine;
N-((3-fluoro-2′-(trifluoromethyl)-2,4′-bipyridin-5-yl)methyl)-6-(3-fluorophenyl)-2,7-naphthyridin-1-amine;
N-((2′-fluoro-2,4′-bipyridin-5-yl)methyl)-6-(3-fluorophenyl)-2,7-naphthyridin-1-amine;
6-(3-fluorophenyl)-N-(3-methyl-4-(2-methylpyridin-4-yl)benzyl)-2,7-naphthyridin-1-amine;
4-(5-(((6-(3-fluorophenyl)-2,7-naphthyridin-1-yl)amino)methyl)pyridine-2-yl)thiomorpholine 1,1-dioxide;
N-(4-chlorobenzyl)-6-(2-methylpyridin-4-yl)-2,7-naphthyridin-1-amine;
N-(4-methylbenzyl)-6-(2-methylpyridin-4-yl)-2,7-naphthyridin-1-amine;
6-(2-methylpyridin-4-yl)-N-(pyridin-3-ylmethyl)-2,7-naphthyridin-1-amine;
N-benzyl-2-(3-fluorophenyl)-1,6-naphthyridin-5-amine;
2-(3-fluorophenyl)-N-((2′-methyl-2,4′-bipyridin-5-yl)methyl)-1,6-naphthyridin-5-amine;
N-((2′-methyl-2,4′-bipyridin-5-yl)methyl)-2-(2-methylpyridin-4-yl)-1,6-naphthyridin-5-amine;
N-((6-(3-fluorophenyl)pyridin-3-yl)methyl)-2-(2-methylpyridin-4-yl)-1,6-naphthyridin-5-amine;
N-(4-(2-fluoropyridin-4-yl)benzyl)-2-(2-methylpyridin-4-yl)-1,6-naphthyridin-5-amine;
2-(2-methylpyridin-4-yl)-N-(4-(2-(trifluoromethyl)pyridin-4-yl)benzyl)-1,6-naphthyridin-5-amine;
N-((2′,3-dimethyl-2,4′-bipyridin-5-yl)methyl)-2-(2-methylpyridin-4-yl)-1,6-naphthyridin-5-amine;
N-(biphenyl-4-ylmethyl)-6-(3-fluorophenyl)isoquinolin-1-amine;
N-((2-fluorobiphenyl-4-yl)methyl)-6-(3-fluorophenyl)isoquinolin-1-amine;
N-((2′-methyl-2,4′-bipyridin-5-yl)methyl)-6-phenylisoquinolin-1-amine;
6-(3-chlorophenyl)-N-((2′-methyl-2,4′-bipyridin-5-yl)methyl)isoquinolin-1-amine;
N-(4-(2-methylpyridin-4-yl)benzyl)-6-phenylisoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-4-yl)isoquinolin-1-amine;
6-(6-methylpyridin-3-yl)-N-(4-(2-methylpyridin-4-yl)benzyl)isoquinolin-1-amine;
6-(2-methylpyridin-4-yl)-N-(4-(2-methylpyridin-4-yl)benzyl)isoquinolin-1-amine;
N-(4-(2-methylpyridin-4-yl)benzyl)-6-(pyridin-3-yl)isoquinolin-1-amine;
N-(4-(2-methylpyridin-4-yl)benzyl)-6-(pyrazin-2-yl)isoquinolin-1-amine;
N-(4-(2-methylpyridin-4-yl)benzyl)-6-(pyridazin-4-yl)isoquinolin-1-amine;
N-((2′-methyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyrazin-2-yl)isoquinolin-1-amine;
N-((2′,3-dimethyl-2,4′-bipyridin-5-yl)methyl)-6-(pyridin-2-yl)isoquinolin-1-amine;
N-((2′,3-dimethyl-2,4′-bipyridin-5-yl)methyl)-6-(3-fluorophenyl)isoquinolin-1-amine;
N-((2′,3-dimethyl-2,4′-bipyridin-5-yl)methyl)-6-(5-methylpyridin-3-yl)isoquinolin-1-amine;
N-((2′-methyl-2,4′-bipyridin-5-yl)methyl)-2-phenylpyrido[4,3-b]pyrazin-5-amine;
2-(3-fluorophenyl)-N-(4-(2-methylpyridin-4-yl)benzyl)pyrido[4,3-b]pyrazin-5-amine;
2-(3-fluorophenyl)-N-((2′-methyl-2,4′-bipyridin-5-yl)methyl)pyrido[4,3-b]pyrazin-5-amine;
2-(3-fluorophenyl)-N-(3-methyl-4-(2-methylpyridin-4-yl)benzyl)pyrido[4,3-b]pyrazin-5-amine;
N-(3-fluoro-4-(2-methylpyridin-4-yl)benzyl)-2-(3-fluorophenyl)pyrido[4,3-b]pyrazin-5-amine;
2-(2-methylpyridin-4-yl)-N-(4-(2-methylpyridin-4-yl)benzyl)pyrido[4,3-b]pyrazin-5-amine;
N-((2′-methyl-2,4′-bipyridin-5-yl)methyl)-2-(2-methylpyridin-4-yl)pyrido[4,3-b]pyrazin-5-amine;
N-(3-methyl-4-(2-methylpyridin-4-yl)benzyl)-2-(2-methylpyridin-4-yl)pyrido[4,3-b]pyrazin-5-amine;
N-(3-fluoro-4-(2-methylpyridin-4-yl)benzyl)-2-(2-methylpyridin-4-yl)pyrido[4,3-b]pyrazin-5-amine;
N-((2′,3-dimethyl-2,4′-bipyridin-5-yl)methyl)-6-(pyrazin-2-yl)-2,7-naphthyridin-1-amine;
6-(2-methylmorpholino)-N-(4-(2-methylpyridin-4-yl)benzyl)-2,7-naphthyridin-1-amine;
(S)-6-(2-methylmorpholino)-N-(4-(2-methylpyridin-4-yl)benzyl)-2,7-naphthyridin-1-amine;
(R)-6-(2-methylmorpholino)-N-(4-(2-methylpyridin-4-yl)benzyl)-2,7-naphthyridin-1-amine;
1-(4-(8-(4-(2-methylpyridin-4-yl)benzylamino)-2,7-naphthyridin-3-yl)piperazin-1-yl)ethanone;
6-(1H-imidazol-1-yl)-N-(4-(2-methylpyridin-4-yl)benzyl)-2,7-naphthyridin-1-amine;
6-(4-methyl-1H-imidazol-1-yl)-N-(4-(2-methylpyridin-4-yl)benzyl)-2,7-naphthyridin-1-amine;
N-(4-(2-methylpyridin-4-yl)benzyl)-6-(1H-tetrazol-5-yl)-2,7-naphthyridin-1-amine;
6-(5-methyl-1,3,4-oxadiazol-2-yl)-N-(4-(2-methylpyridin-4-yl)benzyl)-2,7-naphthyridin-1-amine;
6-(1-methyl-1H-pyrazol-3-yl)-N-(4-(2-methylpyridin-4-yl)benzyl)-2,7-naphthyridin-1-amine;
N-(4-(2-methylpyridin-4-yl)benzyl)-6-(thiazol-5-yl)-2,7-naphthyridin-1-amine;
N-(4-(2-methylpyridin-4-yl)benzyl)-6-(oxazol-5-yl)-2,7-naphthyridin-1-amine;
N-((2′,3-dimethyl-2,4′-bipyridin-5-yl)methyl)-6-(5-methylpyridin-3-yl)-2,7-naphthyridin-1-amine;
N-((2′,3-dimethyl-2,4′-bipyridin-5-yl)methyl)-6-(2-methylpyridin-4-yl)-2,7-naphthyridin-1-amine;
N-((3-fluoro-2′-methyl-2,4′-bipyridin-5-yl)methyl)-6-(2-methylpyridin-4-yl)-2,7-naphthyridin-1-amine;
N-((2′,3-dimethyl-2,4′-bipyridin-5-yl)methyl)-6-(5-fluoropyridin-3-yl)-2,7-naphthyridin-1-amine;
N-(3-methyl-4-(2-methylpyridin-4-yl)benzyl)-6-(pyrazin-2-yl)-2,7-naphthyridin-1-amine;
N-(3-fluoro-4-(2-methylpyridin-4-yl)benzyl)-6-(pyrazin-2-yl)-2,7-naphthyridin-1-amine;
methyl 4-(8-(4-(2-methylpyridin-4-yl)benzylamino)-2,7-naphthyridin-3-yl)piperazine-1-carboxylate;
4-(8-(4-(2-methylpyridin-4-yl)benzylamino)-2,7-naphthyridin-3-yl)piperazin-2-one;
2-(4-(8-(4-(2-methylpyridin-4-yl)benzylamino)-2,7-naphthyridin-3-yl)piperazin-1-yl)acetonitrile;
2-methyl-4-(4-((6-(2-methylpyridin-4-yl)-2,7-naphthyridin-1-ylamino)methyl)phenyl)pyridine 1-oxide;
6-(2-chloropyridin-4-yl)-N-((2′,3-dimethyl-2,4′-bipyridin-5-yl)methyl)-2,7-naphthyridin-1-amine;
6-(2-chloropyridin-4-yl)-N-(4-(2-methylpyridin-4-yl)benzyl)-2,7-naphthyridin-1-amine;
2-(2-methylpyridin-4-yl)-5-((6-(2-methylpyridin-4-yl)-2,7-naphthyridin-1-ylamino)methyl)benzonitrile;
N-(3-methoxy-4-(2-methylpyridin-4-yl)benzyl)-6-(2-methylpyridin-4-yl)-2,7-naphthyridin-1-amine;
N-((3-chloro-2′-methyl-2,4′-bipyridin-5-yl)methyl)-6-(2-methylpyridin-4-yl)-2,7-naphthyridin-1-amine;
2′-methyl-5-((6-(2-methylpyridin-4-yl)-2,7-naphthyridin-1-ylamino)methyl)-2,4′-bipyridine-3-carbonitrile; and N-(4-(2-(difluoromethyl)pyridin-4-yl)benzyl)-6-(2-methylpyridin-4-yl)-2,7-naphthyridin-1-amine;
N-[5-(3-fluorophenyl)pyridin-2-yl]-2-[5-methyl-6-(pyridazin-4-yl)pyridin-3-yl]acetamide;
2-[5-methyl-6-(2-methylpyridin-4-yl)pyridin-3-yl]-N-[5-(pyrazin-2-yl)pyridin-2-yl]acetamide (LGK974);
N-(2,3′-bipyridin-6′-yl)-2-(2′,3-dimethyl-2,4′-bipyridin-5-yl)acetamide;
N-(5-(4-acetylpiperazin-1-yl)pyridin-2-yl)-2-(2′-methyl-3-(trifluoromethyl)-2,4′-bipyridin-5-yl)acetamide;
N-(5-(4-acetylpiperazin-1-yl)pyridin-2-yl)-2-(2′-fluoro-3-methyl-2,4′-bipyridin-5-yl)acetamide;
2-(2′-fluoro-3-methyl-2,4′-bipyridin-5-yl)-N-(5-(pyrazin-2-yl)pyridin-2-yl)acetamide;
or a pharmaceutically acceptable salt thereof.

8. The method off claim 7, wherein said compound is N-(3-fluoro-4-(2-fluoropyridin-4-yl)benzyl)-6-(2-methylpyridin-4-yl)-2,7-naphthyridin-1-amine.

9. The method of claim 1, wherein the therapeutically effective amount of the compound is about 0.01 to 20 mg/kg per body weight at daily dosages.

10. The method of claim 9, wherein the therapeutically effective amount of the compound from about 0.5 mg to about 1000 mg for humans.

11. The method of claim 1, wherein said cancer is colorectal cancer, gastric cancer, liver cancer, esophageal cancer, intestinal cancer, bile duct cancer, pancreatic cancer, endometrial cancer, or prostate cancer.

12. A method for detecting a biomarker correlated with a cancer susceptible to treatment with an antagonist of Porcupine, the method comprising:
(a) isolating a biological sample from a subject having cancer;
(b) performing an assay on said biological sample to detect the presence of an R-spondin fusion comprising
(1) an HNF4G-Rspo2e2 fusion;
(2) a PTPRKe13-Rspo3e2 fusion; or
(3) a PTPRKe6X-Rspo3e2 fusion.

13. The method of claim 12, detecting the presence of an R-spondin fusion comprises detecting a R-spondin mRNA expression level that is higher than the R-spondin mRNA expression level in a control subject that has been determined not to have a R-spondin fusion.

14. The method of claim 12, wherein:
1) said HNF4G-Rspo2e2 fusion comprises a junction sequence of SEQ ID NO.:67;
2) said PTPRKe13-Rspo3e2 fusion comprises a junction sequence of SEQ ID NO.:61; or
3) said PTPRKe6X-Rspo3e2 fusion comprises a junction sequence of SEQ ID NO.:60.

15. The method of claim 12, wherein said Rspondin is Rspo2 or Rspo3, and said fusion gene is overexpressed in comparision to the Rspondin that is not fused to another gene.

16. The method of 12, wherein said cancer is colorectal cancer, gastric cancer, liver cancer, esophageal cancer, intestinal cancer, bile duct cancer, pancreatic cancer, endometrial cancer, or prostate cancer.

17. A method of treating cancer is a subject in need thereof comprising:
(1) obtaining results of an assay to detect biomarker correlated with a cancer susceptible to treatment with an antagonist of Porcupine, the method comprising:
(a) isolating a biological sample from the subject having cancer;
(b) performing an assay on said biological sample to detect the presence of an R-spondin fusion comprising
(i) an HNF4G-Rspo2e2 fusion;
(ii) a PTPRKe13-Rspo3e2 fusion; or
(iii) a PTPRKe6X-Rspo3e2 fusion;
(2) selecting the subject for treatment with an antagonist of Porcupine if the presence of an R-spondin fusion has been detected; and
(3) administering a pharmaceutical composition comprising a therapeutically effective amount of an antagonist of Porcupine wherein said Porcupine antagonist comprises:
a compound of Formula (I): 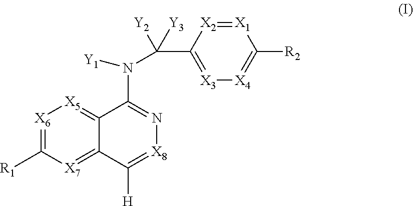
or a physiologically acceptable salt thereof, wherein
X₁, X₂, X₃, X₄, X₅, X₆, X₇, X₈ are independently CR4 or N;
Y₁ is hydrogen or CRa; Y₂, Y₃ are independently hydrogen, halo or CR3;
R₁ is morpholinyl, piperazinyl, quinolinyl, 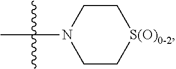
aryl, C₁₋₆ heterocycle, 5 or 6 membered heteroaryl containing 1-2 heteroatoms selected from N, O and S;
R₂ is hydrogen, halo, morpholinyl, piperazinyl, quinolinyl, 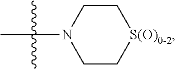
aryl, C₁₋₆ heterocycle, 5 or 6 membered heteroaryl containing 1-2 heteroatoms selected from N, O and S;
R₃ is hydrogen, halo, cyano, C₁₋₆ alkyl, C₁₋₆ alkoxy optionally substituted with halo, amino, hydroxyl, alkoxy or cyano;
R₄ is hydrogen, halo, C₁₋₆alkoxy, —S(O)₂R₅, —C(O)OR₅, —C(O)R₅, —C(O)NR₆R₇, C₁₋₆ alkyl, C₂₋₆ alkenyl or C₂₋₆ alkynyl, each of which can be optionally substituted with halo, amino, hydroxyl, alkoxy or cyano;
R₈, R₆ and R₇ are independently hydrogen, C₁₋₆ alkyl, C₂₋₆ alkenyl or C₂₋₆ alkynyl, each of which may be optionally substituted with halo, amino, hydroxyl, alkoxy or cyano;
a compound of Formula (II): 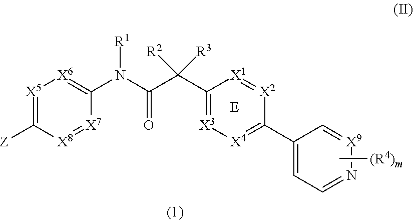
or a physiologically acceptable salt thereof, wherein:
X¹, X², X³ and X⁴ is selected from N and CR⁷;
one of X⁵, X⁶, X⁷ and X⁸ is N and the others are CH;
X⁹ is selected from N and CH;
Z is selected from phenyl, pyrazinyl, pyridinyl, pyridazinyl and piperazinyl;
wherein each phenyl, pyrazinyl, pyridinyl, pyridazinyl or piperazinyl of Z is optionally substituted with an R⁶ group;
R¹, R² and R³ are hydrogen;
m is 1;
R⁴ is selected from hydrogen, halo, difluoromethyl, trifluoromethyl and methyl;
R⁶ is selected from hydrogen, halo and —C(O)Rⁱ⁰; wherein R¹⁰ is methyl; and
R⁷ is selected from hydrogen, halo, cyano, methyl and trifluoromethyl.
